SEQUENCE LISTING (12 sequences)

SEQ ID NO: 1 - protein, 13 aa, Artificial sequence
  Synthetic peptide_Gap26
VCYDKSFPISHVR

SEQ ID NO: 2 - protein, 20 aa, Artificial sequence
  Synthetic peptide_TAT-gap19
YGRKKRRQRRRKQIEIKKFK

SEQ ID NO: 3 - DNA, 20 bp, Artificial sequence
  Synthetic primer_inositolpolyphosphate-1- phosphatase forward primer
ctcctgctctgtcctcatcc

SEQ ID NO: 4 - DNA, 20 bp, Artificial sequence
  Synthetic primer_inositolpolyphosphate-1- phosphatase reverse primer
ctcccggaggatatctgaca

SEQ ID NO: 5 - DNA, 23 bp, Artificial sequence
  Synthetic primer_phosphoglycerate kinase 1 forward primer
caagaagtatgctgaggctgtca

SEQ ID NO: 6 - DNA, 21 bp, Artificial sequence
  Synthetic primer_phosphoglycerate kinase 1 reverse primer
caaatacccccacaggaccat

SEQ ID NO: 7 - DNA, 20 bp, Artificial sequence
  Synthetic primer_ Connexin37 forward primer
ggtgggtaagatctggctga

SEQ ID NO: 8 - DNA, 20 bp, Artificial sequence
  Synthetic primer_connexin37 reverse primer
ggccgtgttacactcgaaat

SEQ ID NO: 9 - DNA, 20 bp, Artificial sequence
  Synthetic primer_connexin40 forward primer
cagggaacagatgccaaaac

SEQ ID NO: 10 - DNA, 21 bp, Artificial sequence
  Synthetic primer_connexin40 reverse primer
agttggagaagaagcagccca

SEQ ID NO: 11 - DNA, 18 bp, Artificial sequence
  Synthetic primer_connexin43 forward primer
ctgagtgcctgaacttgc

SEQ ID NO: 12 - DNA, 19 bp, Artificial sequence
  Synthetic primer_connexin43 reverse primer
actgacagccacaccttcc

(19) United States
(12) Patent Application Publication
(10) Pub. No.: US 2020/0384072 A1
(43) Pub. Date: Dec. 10, 2020

(54) THERAPY OF IONIZING RADIATION-INDUCED DISORDERS

(71) Applicants: SCK-CEN, Brussel (BE); Universiteit Gent, Gent (BE)

(72) Inventors: Luc LEYBAERT, Bachte-Maria-Leerne (BE); An AERTS, Oud-Turnhout (BE); Elke DECROCK, Deinze (BE); Raghda RAMADAN, Mol (BE); Delphine HOORELBEKE, Niel (BE); Sarah BAATOUT, Geel (BE)

(21) Appl. No.: 16/432,483

(22) Filed: Jun. 5, 2019

(51) Int. Cl. A61K 38/17; A61P 39/00

(52) U.S. Cl. CPC A61K 38/1709; A61P 39/00

(57) ABSTRACT

The present invention relates to an agent for inhibiting a connexin protein, connexin hemichannel or connexin gap junction, for use in the therapy of one or more ionizing radiation-induced disorders.

### TECHNICAL FIELD OF THE INVENTION

The present invention relates to the therapy of ionizing radiation-induced disorders and in particular to agents for use in said therapy.

### REFERENCE TO SEQUENCE LISTING SUBMITTED VIA EFS-WEB

This application includes an electronically submitted sequence listing in .txt format. The .txt file contains a sequence listing entitled “19828-365-Sequence_Listing_ST25.txt” created on Nov. 27, 2019 and is 2.94 kilobytes in size. The sequence listing contained in this .txt file is part of the specification and is hereby incorporated by reference herein in its entirety.

### BACKGROUND OF THE INVENTION

Connexins (Cx), or gap junction proteins, are structurally related transmembrane proteins that assemble to form hemichannels and gap junctions that function as channels connecting neighbouring cells. They are ubiquitous proteins expressed in almost all vertebrate cells, are crucial for normal organ and tissue function and play prominent roles in the normal functioning of the heart, brain, liver, vascular system, and other organs and tissues. Endothelial cells, the interior lining of blood vessels and cardiac valves, express three main Cx isotypes, namely Cx37, Cx40 and Cx43. However, gap junctions and hemichannels are an underexplored pharmacological target; among others, because of the limited possibilities of specifically targeting hemichannels versus gap junctions. Moreover, systemic administration of drugs that inhibit gap junctions have considerable potential side effects on cardiac pump function and may disturb heart rhythm. Connexins in cardio- and neurovascular health/disease and therapeutic strategies targeting connexins were recently reviewed by Leybaert et al. (LEYBAERT, Luc, et al. Connexins in cardiovascular and neurovascular health and disease: pharmacological implications. Pharmacological reviews, 2017, 69.4: 396-478.) and Laird and Lampe (LAIRD, Dale W.; LAMPE, Paul D. Therapeutic strategies targeting connexins. Nature Reviews Drug Discovery, 2018, 17.12: 905.).

Connexins assemble in the cell membrane to form hemichannels. In turn, the docking of two connexin hemichannels of adjacent cells leads to the formation of a gap junction. The latter functions as a channel connecting neighbouring cells. ‘Free’ hemichannels not incorporated into gap junctions are normally closed, but may open under conditions that include ischemia, inflammation, electrical stimulation, exposure to reactive oxygen species or nitric oxide, and elevation of the intracellular cytoplasmic calcium concentration.

Uncontrolled opening of hemichannels, a condition sometimes referred to as “leaky hemichannels”, has been reported in various different pathological conditions including cardiac ischemia/reperfusion, stroke, spinal cord injury, pain hypersensitivity, retinal disease, delayed wound healing and inflammatory diseases. In particular, increased hemichannel opening may lead to the release of essential signaling and metabolic molecules from the cell with a molecular weight of up to about 1.5 kDa, thereby compromising cell function. Examples of released molecules are ATP, NAD+, glutamate, prostaglandin E2 and others. Increased hemichannel opening also facilitates substance entry into the cell, as is the case for sodium and calcium ions, leading to calcium ion overload. Because hemichannels are poorly-selective channels, they pass charge-carrying ions and thereby conduct current over the membrane that affects electrical cell functioning, as is the case in the heart.

Breast cancer represents one of the most common malignancies in women with over 1.4 million cases diagnosed annually worldwide. Adjuvant radiotherapy is a standard therapy for breast cancer treatment after conservative surgery and mastectomy. While radiotherapeutic treatment consists of targeted and precise application of radiation beams, exposure of surrounding healthy tissue is inevitable. Exposure of healthy tissue, in particular the heart, to ionizing radiation (IR) often increases the risk for the development of cardiovascular diseases (CVD), especially atherosclerosis. Although modern radiotherapy techniques reduce the volume of the heart and major coronary vessels exposed to high doses of IR, some exposure is often unavoidable, especially in the case of left-sided breast cancer, in which case the dose received by the heart area is in the order of about 6.6 Gy (compared to 2.9 Gy for right-sided breast cancer). A population-based case-control study in women who underwent radiotherapy for breast cancer indicated a significant increase of 7.4% in the rate of major coronary events (i.e. myocardial infarction, coronary revascularization, or death from ischemic heart disease) per increase of 1 Gy in the cardiac exposure dose, without apparent threshold.

In addition to breast cancer, exposure of the cardio- and/or neurovascular system to IR may occur during cancer radiotherapy for e.g. head-and-neck cancer, Hodgkin's lymphoma and oesophageal cancer. Although radiotherapy is an effective treatment for most tumor types, growing evidence indicate a link between IR exposure—at high and medium doses (>0.5 Gy) but also at much lower doses—and, for example, atherosclerosis development. Atherosclerosis is a progressive inflammatory disease of the arterial wall that is initiated with damage to the vascular endothelial cells. In addition, in the brain, IR induces the breakdown of the blood-brain barrier through its effects on brain microvascular endothelial cells, which are most vulnerable to radiation exposure. Moreover, cellular and molecular changes induced by ionizing radiation occur not only in directly irradiated cells, but can also be transferred to adjacent non-irradiated cells; a process known as ‘the bystander effect’. However, the underlying cellular and molecular mechanisms for these conditions have not been fully understood, possibly resulting in improper radiation protection.

As such, there is a need for radioprotective measures to protect the cardio- and/or neurovascular system of a treated patient from radiation-induced secondary health effects. However, at present, there is no radioprotective compound that is clinically approved therefor.

### SUMMARY OF THE INVENTION

It is an object of the present invention to provide good agents for use in the therapy of ionizing radiation-induced disorders and/or the therapy of atherosclerosis. It is a further object of the present invention to provide good uses and methods for use of said agents. This objective is accomplished by agents, pharmaceutical compositions and methods according to the present invention.

It is an advantage of embodiments of the present invention that an effective therapy for ionizing radiation-induced diseases can be formulated. It is a further advantage of embodiments of the present invention that good medical targets for said therapy, as wells as effective agents for targeting said targets, can be identified.

It is an advantage of embodiments of the present invention that a variety of ionizing radiation-induced diseases can be addressed, including cardiovascular disorders (e.g. atherosclerosis), neurovascular disorders (e.g. disturbed blood-brain barrier function) and/or neurodegenerative disorders.

It is an advantage of embodiments of the present invention that side effects of radiotherapy (e.g. thoracic or head-and/or-neck radiotherapy) can be mitigated and/or prevented. It is a further advantage of embodiments of the present invention that this can be achieved both for a patient of the radiotherapy as for assisting medical personnel.

It is an advantage of embodiments of the present invention that a particular isotype (e.g. Cx43) of connexin proteins and/or hemichannels and/or gap junctions can be targeted in a specific manner with respect to another isotype thereof.

It is an advantage of embodiments of the present invention that connexin hemichannels can be targeted in a specific manner with respect to connexin gap junctions.

In a first aspect, the present invention relates to an agent for inhibiting a connexin protein, connexin hemichannel or connexin gap junction, for use in the therapy of one or more ionizing radiation-induced disorders.

In embodiments, at least one of the one or more ionizing radiation-induced disorders may be a cardiovascular disorder, a neurovascular disorder or a neurodegenerative disorder.

In embodiments, the ionizing radiation-induced disorder may be an ionizing radiation-induced atherosclerosis.

In embodiments, the therapy may be for reducing side-effects from a radiotherapy.

In embodiments, the radiotherapy may be a thoracic radiotherapy or a head-and/or-neck radiotherapy.

In embodiments, the connexin protein may be a Cx43.

In embodiments, the connexin hemichannel may be a Cx43 hemichannel.

In embodiments, the connexin gap junction may be a Cx43 gap junction.

In embodiments, the agent may be for inhibiting the connexin hemichannel in a specific manner with respect to a corresponding connexin gap junction.

In embodiments, the agent may be a connexin-targeting molecule or a hemichannel inhibitor.

In embodiments, the agent may be a Gap19-based compound, an L2-based compound or a peptide5-based compound.

In a second aspect, the present invention relates to an agent for inhibiting a connexin protein, connexin hemichannel or connexin gap junction, for use in the therapy of an atherosclerosis.

In preferred embodiments, the agent may be for inhibiting the connexin hemichannel.

In a third aspect, the present invention relates to a pharmaceutical composition comprising an agent according to any embodiment of the first or second aspect or a pharmaceutical composition.

In embodiments, the pharmaceutical composition may further comprise a pharmaceutically acceptable carrier.

In embodiments, any feature of any embodiment of the third aspect may independently be as correspondingly described for any embodiment of any of the other aspects.

In a fourth aspect, the present invention relates to a method of therapy of an ionizing radiation-induced disorder, comprising: (a) inhibiting a connexin protein, connexin hemichannel or connexin gap junction in a subject, and (b) exposing the subject to radiation; wherein step a is performed during and/or after step b.

In embodiments, step a may comprise administering an agent according to any embodiment of the first or second aspect or a pharmaceutical composition according to any embodiment of the third aspect.

In embodiments, the agent and/or pharmaceutical composition may be administered before and/or during and/or after the radiation exposure.

In a fifth aspect, the present invention relates to a use of an agent according to any embodiment of the first or second aspect in a method of therapy of an ionizing radiation-induced disorder.

In embodiments, the method of therapy may be a method of therapy according to any embodiment of the fourth aspect.

Particular and preferred aspects of the invention are set out in the accompanying independent and dependent claims. Features from the dependent claims may be combined with features of the independent claims and with features of other dependent claims as appropriate and not merely as explicitly set out in the claims.

Although there has been constant improvement, change and evolution of products and techniques in this field, the present concepts are believed to represent substantial new and novel improvements, including departures from prior practices, resulting in the provision of more efficient, stable and reliable products and techniques of this nature.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic overview of pathways related to embodiments of the present invention.

FIG. 2 is a table listing forward and reverse primers used to determine gene expression levels via RT-qPCR. RT-qPCR: reverse transcription quantitative polymerase chain reaction, INPP: inositolpolyphosphate-1-phosphatase, PGK1: phosphoglycerate kinase 1, Cx: connexin, FW: forward, RV: reverse.

FIG. 3 is a table listing primary antibodies, secondary antibodies and blocking buffer used for western blot analysis. Cx: connexin, NFDM: non-fat dry milk, BSA: bovine serum albumin.

FIG. 4 to FIG. 13 depict bar graphs showing the effect of single and fractionated irradiation on Cx37, Cx40 and Cx43 gene expression. FIG. 4 to FIG. 9 show gene expression of Cx37, (FIG. 4 and FIG. 5), Cx40 (FIG. 6 and FIG. 7) and Cx43 (FIG. 8 and FIG. 9) at 6 h, 24 h, 48 h, 72 h, 7 d or 14 d after a single X-ray exposure (0.1, 0.5 and 5 Gy) in TICAE (FIG. 4, FIG. 6 and FIG. 8) and TIME cells (FIG. 5, FIG. 7 and FIG. 9). FIG. 10 to FIG. 13 show gene expression of Cx43 at 24 h (FIG. 10 and FIG. 11) and 7 d (FIG. 12 and FIG. 13) after single or fractionated irradiation in TICAE (FIG. 10 and FIG. 12) and TIME cells (FIG. 11 and FIG. 13). Fractionated irradiation involved three consecutive X-rays doses (0.033 and 1.67 Gy/fraction/day), leading to cumulative doses of 0.1 and 5 Gy. Data were analyzed with a nonparametric Mann-Whitney T-test. Values represent average ±SEM of 5 biological replicates, except for 6 h p.i. where 4 biological replicates were used. FIG. 4-FIG. 9: * indicates for a given time point the statistical difference of gene expression after a dose of single irradiation compared to the respective normalized 0 Gy controls at the same time point, FIG. 10-FIG. 13: ⋅ indicates for a given time point the statistical difference of gene expression after a dose of fractionated irradiation compared to the respective normalized 0 Gy controls at the same time point, FIG. 10-FIG. 13: * indicates the statistical difference between fold changes of gene expression after a given radiation dose and a given time of single and fractionated irradiation compared to the respective normalized 0 Gy controls at the same time point. */⋅: p<0.05; **/⋅⋅: p<0.01; ***/⋅⋅⋅: p<0.0001. Cx, connexin; TICAE, Telomerase Immortalized human Coronary Artery Endothelial cells; TIME, Telomerase Immortalized human Microvascular Endothelial cells; p.i, post irradiation; h, hours; d, days; SEM, standard error of mean.

FIG. 14, FIG. 15, FIG. 18, FIG. 19 and FIG. 22 to FIG. 27 depict bar graphs showing the effect of single and fractionated irradiation on Cx40, Cx43 and pCx43 protein levels. Cx40 (FIG. 14 and FIG. 15), Cx43 (FIG. 18 and FIG. 19) and pCx43 (FIG. 22 and FIG. 23) protein levels were assessed 6 h, 24 h, 48 h, 72 h, 7 d and 14 d after a single X-ray exposure (0.1, 0.5 and 5 Gy) in TICAE (FIG. 14, FIG. 18 and FIG. 22) and TIME cells (FIG. 15, FIG. 19 and FIG. 23) relative to 0 Gy controls. FIG. 24 to FIG. 27 show single and fractionated irradiation 24 h (FIG. 24 and FIG. 25) and 7 d (FIG. 26 and FIG. 27) post irradiation on Cx43 protein level in TICAE ((FIG. 24 and FIG. 26) and TIME cells (FIG. 25 and FIG. 27). Data were analyzed with a nonparametric Mann-Whitney T-test. Values represent average ±SEM of 4-6 biological replicates. FIG. 14-FIG. 23: * indicates the statistical differences compared to the respective 0 Gy controls at the same time point, FIG. 24-FIG. 27: * indicates the statistical differences between single and fractionated irradiation for the same radiation dose. ⋅ indicates the statistical differences for either single or fractionated irradiation compared to their respective 0 Gy controls */⋅: p<0.05; **/⋅⋅: p<0.01; ***/⋅⋅⋅: p<0.0001.

FIG. 16, FIG. 17, FIG. 20 and FIG. 21 depict gel electrophoresis results showing the effect of single and fractionated irradiation on Cx40 and Cx43 protein levels. Cropped blots are represented in respective accordance with each of FIG. 14, FIG. 15, FIG. 18 and FIG. 19 and full-length blots are reported in FIG. 72 to FIG. 83. All gels were run following the same experimental conditions (see methods for details). Hela cells overexpressing Cx43 or Cx40 were used as a positive control for assessing protein levels of Cx43 or Cx40, respectively. Signals were normalized to the corresponding vinculin signal of the same membrane and quantified densitometrically using Bio1D analysis software.

FIG. 28, FIG. 30 and FIG. 32 to FIG. 35 depict bar graphs and showing single and fractionated radiation exposure inducing an increase in gap junctional communication. The area of diffusion of the 6-CF dye, representing gap junctional communication was assessed after single irradiation exposure in TICAE (FIG. 28) and TIME (FIG. 30) cells, and after fractionated irradiation exposure in TICAE cells (FIG. 32) and TIME cells. (FIG. 34). FIG. 33 and FIG. 35 show the results for single and fractionated irradiation exposure in respectively TICAE and TIME cells in a side-by-side comparison. Carbenoxolone was used as a control. Data were analyzed with a nonparametric Mann-Whitney T-test. Values represent average ±SEM of five to six biological replicates. * indicates the statistical differences compared to the respective 0 Gy controls at the same time point, and the statistical differences between single and fractionated irradiation for the same radiation dose. ⋅ indicates the statistical differences for either single or fractionated irradiation compared to their respective 0 Gy controls. */⋅: p<0.05; **/⋅⋅: p<0.01. 6-CF, 6-carboxyfluorescein; GJ, gap junction.

FIG. 29 and FIG. 31 depict fluorescence microscopy images in respective accordance with FIG. 28 and FIG. 30.

Error! Reference source not found. to FIG. 45 depicts bar graphs showing a radiation-induced increase in extracellular ATP and the effect of TAT-Gap19 after single and fractionated irradiation. ATP release was measured 1 h (FIG. 36 and FIG. 39, 6 h (FIG. 37 and FIG. 40) and 72 h (FIG. 38 and FIG. 41) after single radiation exposure (0.1 and 5 Gy) in (a) TICAE cells (FIG. 36, FIG. 37 and FIG. 38) and TIME cells (FIG. 39, FIG. 40 and FIG. 41) and 72 h after fractionated irradiation exposure in TICAE cells (FIG. 42) and TIME cells (FIG. 44). FIG. 43 and FIG. 45 show the results 72 h after single and fractionated irradiation exposure in respectively TICAE and TIME cells in a side-by-side comparison. TAT-Gap19 was used to block Cx43 hemichannels. Data were analyzed with a nonparametric Mann-Whitney T-test. Values represent average ±SEM of 6-8 biological replicates. * indicates the statistical differences compared to the respective 0 Gy controls, and statistical difference between single and fractionated irradiation for the same radiation doses. ⋅ indicates the statistical difference compared to the respective control condition (not treated with TAT-Gap 19), FIG. 42-FIG. 45 ⋅ indicates the statistical differences for either single or fractionated irradiation compared to their respective 0 Gy controls; */⋅: p<0.05; **/⋅⋅: p<0.01; ***/⋅⋅⋅: p<0.0001.

FIG. 46 to FIG. 55 depict bar graphs showing radiation-induced PI dye uptake responses and the effect of TAT-Gap19 after single and fractionated radiation exposure. FIG. 46 to FIG. 48 show dye uptake 1 h (FIG. 46), 6 h (FIG. 47) and 72 h (FIG. 48) p.i. in TICAE cells. (FIG. 49 to FIG. 51 show dye uptake responses in 1 h (FIG. 49), 6 h (FIG. 50) and 72 h (FIG. 51) p.i. TIME cells. FIG. 52 shows dye uptake 72 h after fractionated irradiation in TICAE cells and FIG. 53 shows the same results in a side-by-side comparison with the dye uptake 72 h after single irradiation. FIG. 54 shows dye uptake responses to fractionated irradiation (72 h) in TIME cells and FIG. 55 shows the same results in a side-by-side comparison with the dye uptake 72 h after single irradiation. Data were analyzed with a nonparametric Mann-Whitney T-test. Values represent average ±SEM of 6 biological replicates. * indicates the statistical differences compared to the respective 0 Gy controls, and statistical difference between single and fractionated irradiation for the same radiation doses. ⋅ indicates the statistical difference compared to the respective control condition (not treated with TAT-Gap 19), FIG. 52-FOG. 55 ⋅ indicates the statistical differences for either single or fractionated irradiation compared to their respective 0 Gy controls. */⋅: p<0.05; **/⋅⋅: p<0.01.

FIG. 56 depicts a principal component analysis (PCA) incorporating both Cx40 and Cx43 gene expression and protein level in TICAE and TIME cells from 6 h to 14 d time period and radiation doses of 0.1, 0.5 and 5 Gy.

FIG. 57 is a table outlining single irradiation effects on gap junction and hemichannel function. *Only TAT-Gap19 inhibitable responses shown, -: indicates no significant effect, ⬆: indicates an increase.

FIG. 58 is a table outlining distinct effects of fractionated irradiation versus single irradiation found within the present invention. -: indicates no significant effect, ⬆: indicates an increase, ⬇: indicates a decrease.

FIG. 59 to FIG. 66 depict bar graphs showing the effect of single and fractionated irradiation on Cx37 and Cx40 gene expression at 24 h and 7 d post irradiation exposure. Cx37 (FIG. 59 to FIG. 62) and Cx40 ((FIG. 63 to FIG. 66) relative gene expression (fold change) were assessed at 24 h (FIG. 59, FIG. 60, FIG. 63 and FIG. 64) and 7 d (FIG. 61, FIG. 62, FIG. 65 and FIG. 66) after single and fractionated X-ray exposure (0.1 and 5 Gy) in TICAE (FIG. 59, FIG. 61, FIG. 63 and FIG. 65) and TIME cells (FIG. 60, FIG. 62, FIG. 64 and FIG. 66). Comparison between single and fractionated irradiation is limited to the radiation response after normalizing the controls. Data were analyzed with a nonparametric Mann-Whitney T-test. Values represent average ±SEM of five biological replicates. * indicates the statistical differences between single and fractionated irradiation for the same radiation dose. ⋅ indicates the statistical differences for either single or fractionated irradiation compared to their respective 0 Gy controls. */⋅: p<0.05; **/⋅⋅: p<0.01.

FIG. 67 to FIG. 70 depict bar graphs showing the effect of single and fractionated irradiation on Cx40 protein levels in TICAE and TIME cells 24 h and 7 d post irradiation. Cx40 protein levels were assessed 24 h (FIG. 67 and FIG. 68) and 7 d (FIG. 69 and FIG. 70) after a single and fractionated X-ray exposure (0.1 and 5 Gy) in TICAE ((FIG. 67 and FIG. 69) and TIME cells (FIG. 68 and FIG. 70). These data were analyzed with a nonparametric Mann-Whitney T-test. The values represent the average ±SEM of five to six biological replicates. * indicates the statistical differences between single and fractionated irradiation for the same radiation dose. ⋅ indicates the statistical differences for either single or fractionated irradiation compared to their respective 0 Gy controls. */⋅: P<0.05; **/⋅⋅: P<0.01.

FIG. 71 depicts a two dimensional principal component analysis (PCA) incorporating Cx40 and Cx43 gene expression and protein level for single and fractionated irradiation at 24 h and 7 d p.i. in TICAE and TIME cells. Such analysis indicated a dose-dependent separation between the radiation doses used (0.1 and 5 Gy) in both single and fractionated irradiation, which significantly shifted the PCA profiles along the positive side of the first component axis (p<0.001), reflecting a dose-dependent response in Cx40 and Cx43 gene expression and protein level after both single and fractionated radiation exposure.

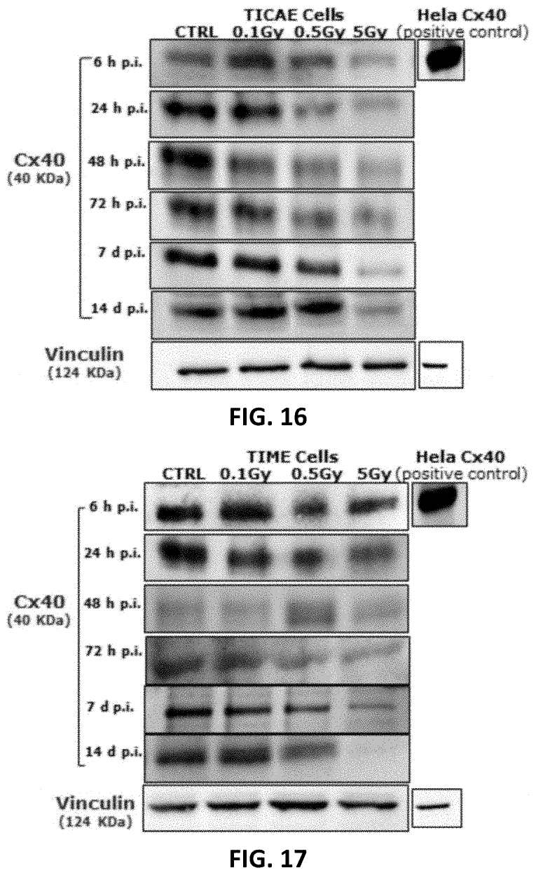

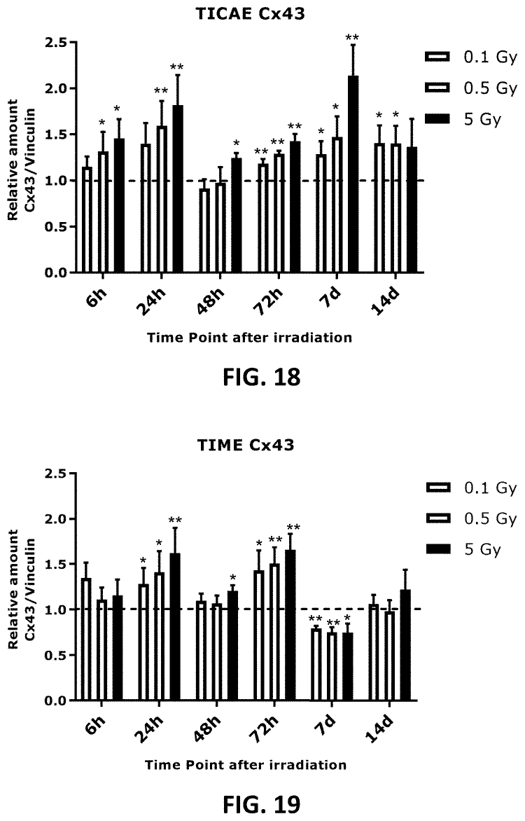

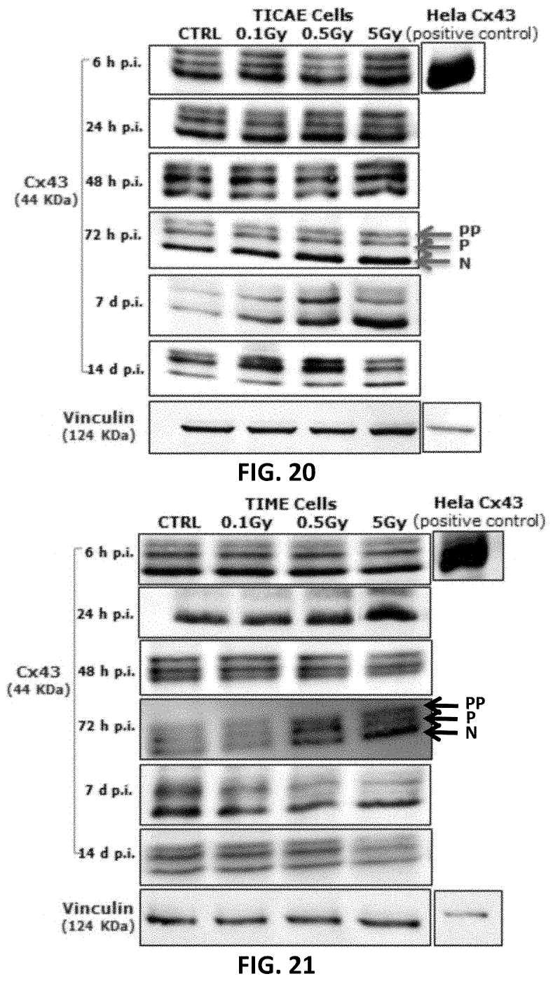

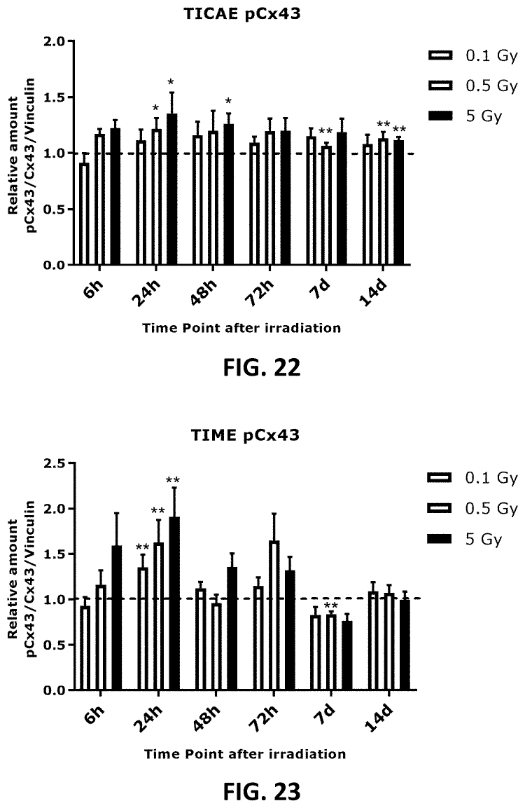

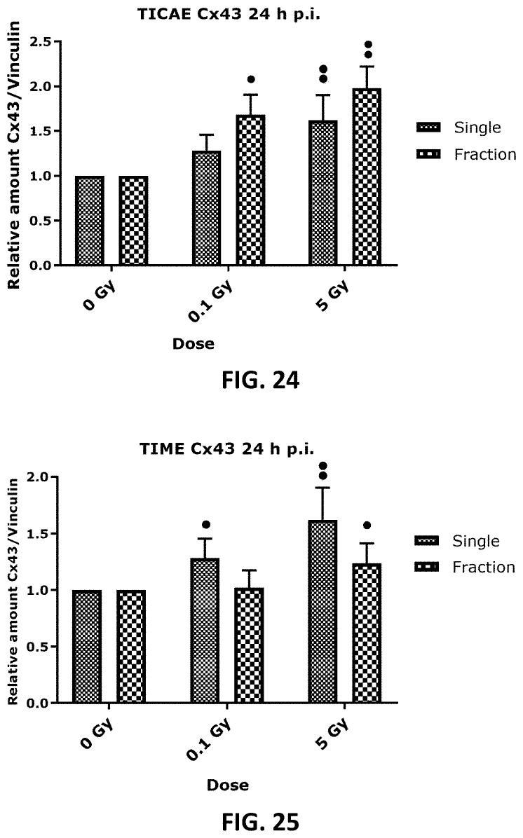

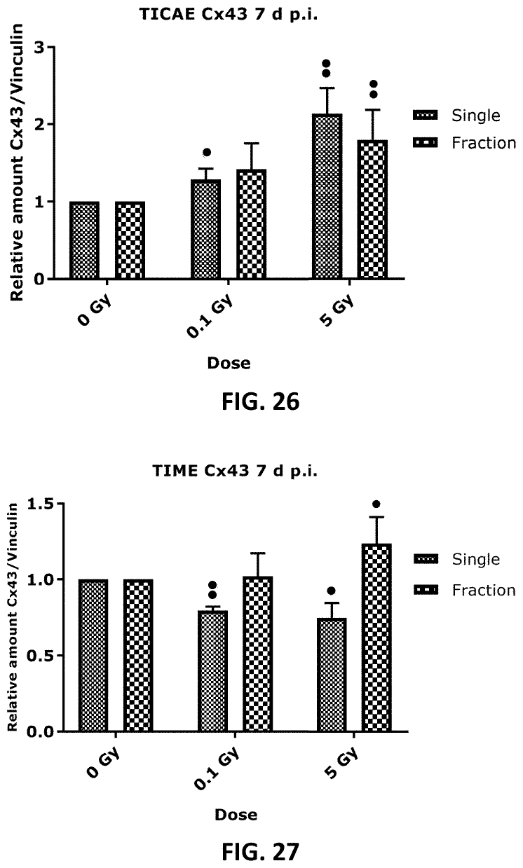

FIG. 72 to FIG. 83 depict full length blots of Cx40 (FIG. 72 to FIG. 77) and Cx43 (FIG. 78 to FIG. 83) in TICAE and TIME cells shown in FIG. 16, FIG. 17, FIG. 20 and FIG. 21. For all the blots, Vinculin was ran on the same gel and the membranes were cut between 75 and 100 KD to image Vinculin and Cx40/CX43 separately. Signals were normalized to the corresponding vinculin signal of the same membrane and quantified densitometrically using Bio1D analysis software. Some gel images were cropped to remove samples that were ran on the same gel, but were not used in FIG. 16, FIG. 17, FIG. 20 and FIG. 21

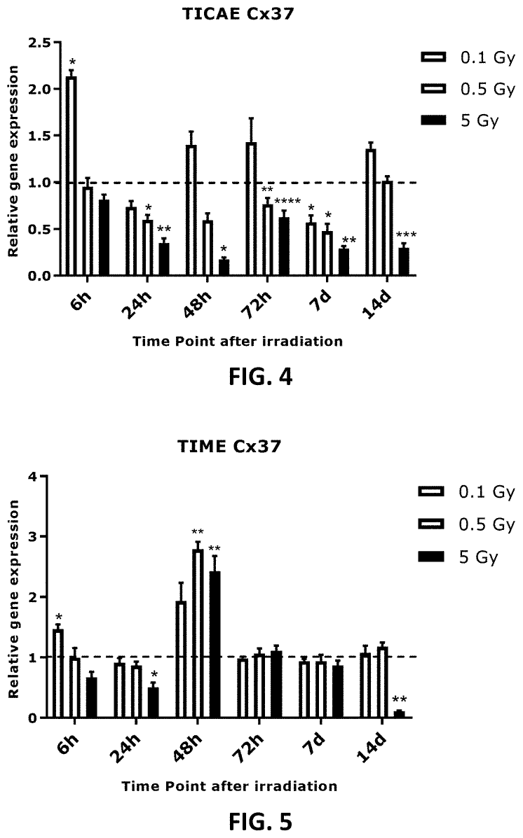

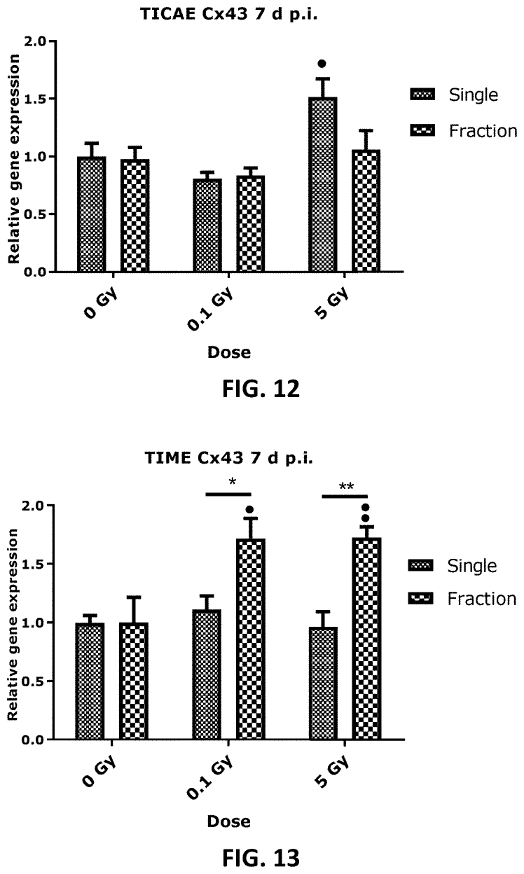

FIG. 84 to FIG. 95 depict bar graphs showing the effect of single and fractionated irradiation on Cx37 (FIG. 84, FIG. 85, FIG. 90 and FIG. 91), Cx40 (FIG. 86, FIG. 87, FIG. 92 and FIG. 93) and Cx43 (FIG. 88, FIG. 89, FIG. 94 and FIG. 95) gene expression at 24 h (FIG. 84 to FIG. 89) and 7 d (FIG. 90 to FIG. 95) post irradiation, in TICAE FIG. 84, FIG. 86, FIG. 88, FIG. 90, FIG. 92 and FIG. 94 and TIME (FIG. 85, FIG. 87, FIG. 89, FIG. 91, FIG. 93 and FIG. 95 without performing normalization of the controls. Normalization of the control of different time points and for radiation regimen (single and fractionated) were performed in FIG. 4 to FIG. 13 and FIG. 54 to FIG. 66 to avoid technical and experimental variations, as the experiments of different time points were not treated at the same time (even though the measurements were presumably carried out under identical circumstances), and they were ran on different qPCR runs. These data were analyzed with a nonparametric Mann-Whitney T-test. The values represent the average ±SEM of five to six biological replicates. * indicates the statistical differences between single and fractionated irradiation for the same radiation dose. ⋅ indicates the statistical differences for either single or fractionated irradiation compared to their respective 0 Gy controls. */⋅: P<0.05; **/⋅⋅: P<0.01.

FIG. 96 AND FIG. 97 depict bar graphs showing radiation-induced ROS production and the effect of TAT-Gap19 in TICAE and TIME cells. FIG. 98 and FIG. 99 depict scatter plots showing radiation-induced ROS production and the effect of TAT-Gap19 in TICAE and TIME cells. FIG. 100 to FIG. 102 depict fluorescence microscopy images showing radiation-induced ROS production and the effect of TAT-Gap19 in TICAE and TIME cells. Intracellular ROS was assessed using CM-H2DCFDA combined with flow cytometry performed at 45 min after irradiation (0.1 and 5 Gy) (FIG. 96 and FIG. 97) or with IncuCyte live cell imaging at three time points (45 min, 2 h and 3 h) after irradiation (FIG. 98 and FIG. 99). FIG. 100 and FIG. 101 show representative images for ROS production at 45 min p.i. in TICAE (FIG. 100) and TIME (FIG. 101) cells, with dark colored cells indicating above threshold ROS signal, FIG. 102 shows representative images for ROS production in TICAE cells in response to tert-Butyl hydroperoxide (tBHP) as a positive control condition; purple colored cells indicate above threshold ROS signal. The values represent the average ±SEM of 15 biological replicates (from four independent experiments) for TICAE cells, and 6 biological replicates for TIME cells in flow cytometry assay, and 16 biological replicates for TICAE and TIME cells in IncuCyte assay. Statistical analysis was done with a nonparametric Mann-Whitney T-test. * indicates statistically significant differences compared to the respective 0 Gy controls. ⋅ indicate statistically significant differences compared to the respective control condition (not treated with TAT-Gap19). *1.: p<0.05; **/⋅⋅: p<0.01; ***/⋅⋅⋅: p<0.0001, ****/⋅ . . . : p<0.00001.

FIG. 103 depicts a scatter plot showing radiation-induced cell death as assessed by Caspase 3/7 activity in TICAE cells from 4 h to 100 h p.i. and the effect of TAT-Gap19. FIG. 104 and FIG. 105 depict scatter plots showing radiation-induced cell death as assessed by Annexin V level in TICAE (FIG. 104) and TIME cells (FIG. 105) from 4 h to 100 h p.i. and the effect of TAT-Gap19. FIG. 106 to FIG. 107 depict bar graphs showing radiation-induced cell death as assessed by 10 kDa dextran fluorescein staining of TIME cells, measured at 6 h (FIG. 106) and 72 h (FIG. 107) p.i and the effect of TAT-Gap19. The values represent the average ±SEM of 6-8 biological replicates; statistical analysis was done with nonparametric two-way ANOVA followed by a Tukey test for Annexin V and Caspase 3/7 activity, and with a nonparametric Mann-Whitney T-test for the dextran fluorescein assay. * indicates statistically significant differences compared to the respective 0 Gy controls. ⋅ indicates statistically significant differences of the TAT-Gap19 group compared to the corresponding respective control condition (not treated with TAT-Gap19. */⋅: p<0.05; **/⋅⋅: p<0.01; ***/⋅⋅⋅p<0.0001, ****/⋅⋅⋅⋅: p<0.00001.

FIG. 108 TO FIG. 131 depict bar graphs showing radiation-induced inflammatory responses and the effect of TAT-Gap19. The response of various inflammatory markers in TICAE cells (FIG. 108 to FIG. 115) and TIME cells (FIG. 116 to FIG. 131) to 0.1 Gy and 5 Gy irradiation conditions is shown. The values represent the average ±SEM of 5-6 biological replicates; data were analyzed with a nonparametric Mann-Whitney T-test. * indicates statistical differences compared to the respective 0 Gy controls. ⋅ indicate statistically significant differences of the TAT-Gap19 group compared to the corresponding responses in the control group (not-treated with TAT-Gap19). */⋅: p<0.05; **/⋅⋅: p<0.01. IL-1β, interleukin 1 beta; IL-6, interleukin 6; IL-8, interleukin 8; ICAM-1, Intracellular Adhesion Molecule; 1 VCAM-1, Vascular cell adhesion protein 1; PECAM-1, Monocyte chemotactic protein-1; TNF-α, Tumor necrosis factor alpha; MCP-1, Platelet endothelial cell adhesion molecule-; CRP, C-reactive protein.

FIG. 132 to FIG. 141 depict bar graphs showing radiation-induced premature endothelial senescence and the effect of TAT-Gap19. Senescence-associated β-galactosidase activity was measured in a CPRG assay at 7 (FIG. 132 and FIG. 135), 9(FIG. 133 and FIG. 136) and 14 d (FIG. 134 and FIG. 137) after radiation exposure (0.1 Gy and 5 Gy) in (a) TICAE cells (FIG. 132 to FIG. 134) and TIME cells FIG. 135 to FIG. 137. IGFBP-7 7 (FIG. 138 and FIG. 140) and GDF-15 (FIG. 139 and FIG. 141) were assessed in TICAE cells and (FIG. 138 and FIG. 139) TIME cells (FIG. 140 and FIG. 141) at 7 d p.i. cells. The values represent the average ±SEM of 16-24 biological replicates in the CPRG assay and of 6 biological replicates in multiplex-based assays. Statistical significance was analyzed with a nonparametric Mann-Whitney T-test. * indicates statistically significant differences compared to the respective 0 Gy controls. ⋅ indicates statistically significant differences of the TAT-Gap19 group compared to the respective control condition (not treated with TAT-Gap19). */⋅: p<0.05; **/⋅⋅: p<0.01, ***/⋅⋅⋅: P<0.001, ****/⋅ . . . : P<0.0001.

FIG. 142 to FIG. 147 depict bar graphs showing radiation-induced endothelial DNA damage and the effect of TAT-Gap19. Gamma H2AX foci (FIG. 142 and FIG. 145), TP53BPI foci (FIG. 143 and FIG. 146) and the colocalized Gamma H2AX/TP53BPI foci (FIG. 144 and FIG. 147) were assessed in TICAE cells (FIG. 142 to FIG. 144) and TIME cells (FIG. 145 to FIG. 147) at 1 h after 0.1 Gy and 5 Gy of X-ray exposure with or without applying TAT-Gap19. FIG. 148 depicts representative wide-field epifluorescence microscopy images showing gamma H2AX foci, TP53BPI foci and colocalized gamma H2AX/TP53BPI foci in DAPI stained nuclei of TICAE cells at 5 Gy dose. The values represent the average ±SEM of 8 biological replicates; statistical significance was analyzed with a nonparametric Mann-Whitney T-test. * indicates statistically significant differences compared to the respective 0 Gy controls. ⋅ indicates statistically significant differences of the TAT-Gap19 group compared to the corresponding responses in the control group (not treated with TAT-Gap19). */⋅: p<0.05; **/⋅⋅: p<0.01, ***/⋅⋅⋅: p<0.001.

FIG. 149 is a scatter plot showing caspase 3/7-dependent cell death in TIME cells after 0.1 Gy and 5 Gy of radiation exposure. Caspase 3/7 activity was assessed in TIME cell after 4 h until 100 h of 0.1 Gy and 5 Gy of IR exposure using IncuCyte live cell imaging. No significant changes in Caspase 3/7 activity were observed for the 0.1 Gy and 5 Gy doses compared to 0 Gy control. Data were analyzed with a nonparametric two-way ANOVA followed by Turky test. The values represent the average ±SEM of 8 biological replicates.

FIG. 150 is a table showing the effect of radiation exposure and TAT-Gap19 on different atherosclerosis inflammatory markers at 24 h, 48 h, 72 h and 7 d post exposure in TICAE and TIME cells. The effect of 0.1 Gy and 5 Gy of IR exposure was compared to 0 Gy controls, while 0 Gy, 0.1 Gy and 5 Gy TAT-Gap19 was compared to the respective radiation dose of the control (IR only) conditions.

FIG. 151 to FIG. 155 depict bar graphs showing the effect of radiation exposure, TAT-Gap19 and TAT alone on IL-6 (FIG. 151), MCP-1 (FIG. 152), IL-1B (FIG. 152), CRP (FIG. 154) and Endothelin-1 (FIG. 155) in TICAE cells at 24 h post exposure. Data were analyzed with a nonparametric Mann-Whitney T-test. The values represent the average ±SEM of 5 biological replicates. * indicates the statistical differences compared to the respective 0 Gy controls. ⋅ indicate the statistical difference compared to the respective radiation dose of the control conditions (un-treated with TAT-Gap19). */⋅: p<0.05; **/⋅⋅: p<0.01. IL-6, interleukin 6; IL-1B, Interleukin 1 beta; MCP-1, Platelet endothelial cell adhesion molecule-1; CRP, C-reactive protein.

FIG. 156 to FIG. 158 and FIG. 160 to FIG. 164 depict fluorescence microscopy images to a model system for investigating bystander signaling in response to local irradiation with X-rays. FIG. 156 shows a control non-irradiated example image of RBE4 cells, an immortalized rat brain endothelial cell line, marked with DAPI nuclear staining and background γ-H2AX staining (×10 objective; scale bar 1 mm). FIG. 157 and FIG. 153 show magnifications showing nuclear detail (×40; scale bar 10 μm). FIG. 160 shows, using a small aperture 3×3 mm collimator for local irradiation, a corresponding image taken 3 h post-irradiation showing high density γ-H2AX foci in the irradiated zone (within dotted line; 20 Gy), and lower density in the surrounding bystander area (DAPI staining; scale bar 1 mm). FIG. 161 to FIG. 164 show nuclear detail magnifications in irradiated and bystander areas (scale bar 10 μm).

FIG. 159 shows a schematic drawing of the aforementioned small aperture 3×3 mm collimator.

FIG. 165 shows a radiosensitive (GafChromic) film placed underneath the cell dish, which was used to locate the irradiated zone (scale bar 1 mm). FIG. 166 shows an overlay of the radiosensitive film and the γ-H2AX staining (scale bar 1 mm).

FIG. 167 depicts a scatter plot showing that the demarcation of the irradiated zone was defined as the full width at two thirds of the maximum radiation intensity (double arrowed line; about 3.4 mm wide) delineating the zone with the highest γ-H2AX foci count (raw counts without background subtraction).

FIG. 168 depicts a scatter plot with the spatial profile of γ-H2AX foci in the bystander area in shaken and non-shaken cell dishes 30 min after irradiation (γ-H2AX counts normalized to 100% at the border of the irradiation). Gamma-H2AX counts averaged over the 1500 to 3000 μm interval were significantly different from each other.

FIG. 169 and FIG. 170 depict scatter plots of the Time dependence of γ-H2AX counts appearance in the irradiated (FIG. 169) and bystander (FIG. 170) areas (γ-H2AX counts relative to the number of nuclei and corrected for background counts in non-irradiated paired cell dishes) for 1 and 20 Gy irradiation. * vs non-irradiated (n=5-11).

FIG. 171 depicts a bar graph showing that gamma-H2AX counts in the bystander area recorded 3 h post-irradiation were not different between RBE4 cells and pBMECs (primary brain microvascular endothelial cells) isolated from C57B16 mice. * vs non-irradiated.

FIG. 172 shows a schematic drawing of a 3×3 mm collimator used for irradiation of cell dishes.

FIG. 173 and FIG. 174 depict fluorescence microscopy images of Gamma-H2AX staining in cell dishes irradiated with the 3×3 mm collimator and treated with vehicle (FIG. 173) or Gap26 (FIG. 174; scale bars 1 mm).

FIG. 175 and FIG. 176 depict bar graphs showing the effect of the connexin channel inhibition on γ-H2AX scores (normalized to vehicle) in RBE4 cells in the irradiated zone (N=8-9) FIG. 175 and in the bystander area (N=8-9) (FIG. 176).

FIG. 177 and FIG. 178 depict bar graphs showing the effect of the connexin channel inhibition on γ-H2AX scores (normalized to vehicle) in RBE4 cells in the irradiated zone (N=4-10) (FIG. 177) and in the bystander area (N=4-10) (FIG. 178)

FIG. 179 depicts a bar graph showing γ-H2AX scores in the bystander area in pBECs derived from C57BL/6 Cx43fl/fl:Tie2-Cre mice (N=12).

FIG. 180 shows a gel electrophoresis result related to FIG. 179.

FIG. 181 depicts a schematic drawing showing irradiation triggering hemichannel opening and ATP release.

FIG. 182 depicts a scatter plot showing broad-beam irradiation triggering ATP release (normalized to control) in RBE4 cells. * vs non-irradiated control (n=6).

FIG. 183 depicts a bar graph summarizing data from the 5 min point 1 Gy irradiation (boxed area in FIG. 182) and illustrating the effect of connexin channel inhibition. * vs vehicle (n=4)

FIG. 184 depicts a schematic drawing of a 3×3 mm collimator used for irradiation.

FIG. 185 depicts a schematic drawing of irradiation triggering propidium iodide dye uptake.

FIG. 186 and FIG. 187 depicts fluorescence microscopy images of propidium iodide dye uptake in the irradiated and bystander areas, without irradiation (FIG. 186) and 5 min post-irradiation with the 3×3 mm collimator (1 Gy, scale bar 1 mm) (FIG. 187).

FIG. 188 depicts a bar graph summarizing data demonstrating radiation induced dye uptake (relative to the number of nuclei and background corrected for signal in non-irradiated cells) that is inhibited by Gap26 in both irradiated and bystander areas.* vs non-irradiated, # vs vehicle, (n=4).

FIG. 189 shows results of patch clamp experiments on HeLa-Cx43 cells demonstrating traces and matching all-point histograms depicting typical Vm-induced (+70 mV, 30 s) Cx43 hemichannel unitary current activity without irradiation and after 1 or 20 Gy irradiation. The histogram illustrates 220 pS and 440 pS peaks that are typical for Cx43 hemichannel opening.

FIG. 190 depicts a scatter plot of the time course of unitary current activities for the conditions explained for FIG. 189. Points represent membrane charge transfer (Qₘ) recorded at different time points after irradiation. Linear regression analysis demonstrates that the slopes increase from 0 to 20 Gy, indicating that hemichannel opening increases with time after radiation exposure.

FIG. 191 depicts a scatter plot of Qₘ summary data for repeated Vm steps to +70 mV (n=5). * vs non-irradiated.

FIG. 192 depicts a schematic drawing of collimator used for broad-beam irradiation, used to show irradiation triggering cytosolic Ca²⁺ dynamics.

FIG. 193 depicts a graph showing broad-beam irradiation of RBE4 cells triggering cytosolic Ca²⁺ oscillations recorded 5 min after 1 Gy irradiation.

FIG. 194 and FIG. 195 depict bar graphs showing the percentage of oscillating cells (FIG. 194) and number of oscillations per cell (FIG. 195) for a non-irradiated samples and after 1 Gy and 20 Gy irradiation. Both the percentage of oscillating cells and the number of oscillations per cell increased upon irradiation. *vs non-irradiated (n=2 cell dishes).

FIG. 196 depicts fluorescence microscopy images of cytosolic Ca²⁺ imaging, demonstrating Ca²⁺ dynamics in response to medium transfer from 1 Gy broad-beam irradiated RBE4 cell dishes to reporter RBE4 cells loaded with fluo-3-AM (scale bar measures 20 μm). Image a shows resting fluo-3 fluorescence; images b to f are AF/F images with b just before medium transfer and subsequent images at the time points indicated.

FIG. 197 depicts a schematic drawing of broad-beam irradiation and medium transfer as used for the experiment corresponding to FIG. 196

FIG. 198 depicts a graph showings time course of cytosolic Ca²⁺ changes upon medium transfer.

FIG. 199 depicts a bar graph of the area under the curve (AUC) of the Ca²⁺ changes, which significantly increased with radiation exposure. * vs non-irradiated (n=3-7).

FIG. 200 and FIG. 201 depict bar graphs of the cytosolic Ca²⁺ increase (in AUC %) when connexin channel inhibitors were added either to the irradiated cell dish (FIG. 200) or recipient cell dish (FIG. 201), which strongly reduced the Ca²⁺ response in the recipient cells (AUC, expressed relative to vehicle). * vs vehicle (n=3-8).

FIG. 202 depicts a schematic drawing of broad-beam irradiation used to showing irradiation increasing ROS production.

FIG. 203 to FIG. 205 depict fluorescence microscopy images after broad-beam irradiation of CM-H2DFDA loaded RBE4 cells measured 5 min after irradiation (non-irradiated, 1 and 20 Gy respectively; scale bar 100 μm); the broad-beam irradiation increasing the fluorescence.

FIG. 206 depicts a bar graph of the quantification of the fluorescence intensity relative to the number of cells (nuclei count) and normalized to the non-irradiated condition, showings significantly increased signal for 1 and 20 Gy irradiation. * vs non-irradiated (n=10-11).

FIG. 207 depicts a schematic drawing of local 3×3 mm beam irradiation experiments to show inhibitors of signaling via Ca²⁺, ROS, NO, ATP and IP₃ inhibit γ-H2AX responses in the irradiated and bystander areas.

FIG. 208 and FIG. 209 depict bar graphs of the effect of cytosolic Ca²⁺-chelation with BAPTA-AM, ROS scavenging with NALC, NO-scavenging with C-PTIO, purinergic P2X antagonism with PPADS and IP3 receptor antagonism with xestospongin C (Xesto C) on γ-H2AX counts (normalized to vehicle) in RBE4 cells in the irradiated zone (FIG. 208) and in the bystander area (FIG. 209). * vs vehicle (n=7-10).

FIG. 210 and FIG. 211 depict bar graphs of the effect of cytosolic Ca²⁺-chelation with BAPTA-AM, ROS scavenging with NALC and NO-scavenging with C-PTIO on γ-H2AX counts (normalized to vehicle) in irradiated (FIG. 210) and bystander (FIG. 211) areas in pBMECs. *vs vehicle (n=4-7).

FIG. 212 depicts a schematic drawing of irradiation-induced ATP release.

FIG. 213 depicts a bar graph showing that BAPTA-AM, NALC and C-PTIO also inhibited irradiation-induced ATP release (normalized to non-irradiated control). * vs vehicle (n=3-6).

FIG. 214 depicts a schematic drawing giving an overview of the electroporation experiment, with indication of the irradiated zone and electroporation zone located in the bystander area, used to show that localized electroporation loading of cells in the bystander zone with cell impermeable inhibitors strongly reduce γ-H2AX scores.

FIG. 215 and FIG. 216 depict fluorescence microscopy images of the electroporation zone in RBE4 cells as visualized by 10 kDa Dextran Texas Red (DTR) (FIG. 215) or Fura Red (FIG. 216) as a fluorescent reporter, immediately after electroporation (0 h time point; size bar 50 μm).

FIG. 217 is table showing the full with at half maximum (FWHM) for both dyes at 0 h and later.

FIG. 218 and FIG. 219 depict fluorescence microscopy images showing representative γ-H2AX foci with indication of the electroporation zone loaded with vehicle solution (FIG. 218) or with BAPTA (FIG. 219) (size bar 1 mm). The dotted line indicates the border of the irradiated area.

FIG. 220 shows a bar graph summarizing data of γ-H2AX counts in the electroporation zone, demonstrating that SOD, BAPTA and BH4-Bcl2 significantly decreased γ-H2AX counts compared to vehicle. * vs vehicle (n=4-5).

FIG. 221 depicts a schematic view of the bystander signal communication network. Ionizing radiation directly interacts with molecules but also indirectly via reactive oxygen species (ROS) and reactive nitrogen species (RNS) produced by ROS interaction with nitric oxide (NO), leading to direct and indirect DNA damage. ROS elevates intracellular Ca²⁺, and Ca²⁺ on its turn triggers ROS; Ca²⁺ also activates the production of NO and IP₃, and the release of ATP that are involved in intracellular and extracellular bystander communication/propagation. IP₃ and Ca²⁺ pass through gap junctions (GJs) while ATP is released via various mechanisms including hemichannels (HCs) that are opened by ROS, Ca²⁺ and NO.

FIG. 222 and FIG. 223 depicts gel electrophoresis results showing connexin expression in brain microvascular endothelial cells namely Western blotting analysis demonstrating Cx43, Cx40 and Cx37 expression in RBE4 cells and primary BMECs. Total protein was determined by sypro staining. Primary BMECs, also expressed the three Cxs. Beta-tubulin was used as a loading control.

FIG. 224 to FIG. 226 depict fluorescence microscopy images of representative immunostainings demonstrating Cx37 (FIG. 224), Cx40 (FIG. 225) and Cx43 (FIG. 226), CD31 and DAPI nuclei (scale bar 80 μm).

FIG. 227 and FIG. 228 depict representative fluorescence microscopy images for determining γ-H2AX counts in the irradiated area, recorded with ×10 objective (FIG. 227) or x63 objective (FIG. 228; scale bar 3 μm).

FIG. 229 depicts a scatter plot showing the relation between low and high magnification analysis of γ-H2AX foci counts in the irradiated zone. The y-axis shows γ-H2AX counts in the irradiated area determined from images recorded with a ×10 objective (scale bar 1 mm) whereas the x-axis shows the percentage of γ-H2AX stained surface area relative to the nuclear surface area based on images acquired with a x63 objective (n=2-3). The relation is initially linear and flattens at higher doses, resulting in an underestimation of the γ-H2AX counts in the irradiated zone for the 20 Gy dose.

FIG. 230 depicts a gel electrophoresis result of Cx43 connexin expression in RBE4 cells.

FIG. 231 depicts a bar graph showing Cx43 expression in RBE4 cells shows a trend to increase following irradiation with 1 and 20 Gy.

FIG. 232 depicts a bar graph showing Cx37 expression is increased in pBECs isolated from C57BL/6 Cx43fl/fl:Tie2-Cre+ mice compared to Cre-control animals (n=4).

FIG. 233 depicts a bar graph of gap junctional coupling studied by scrape loading and dye transfer (SLDT) quantified by the spatial constant of 6-carboxyfluorescein dye spread (normalized to non-irradiated; n=6). Irradiation with 1 Gy did not significantly alter dye spread.

FIG. 234 depicts a bar graph of experiments on C6 WT cells and stably expressing mutant Cx26 with reduced IP3 permeability (C6V84L) (n=7), demonstrating significantly reduced γ-H2AX counts in the irradiated and bystander areas.

FIG. 235 depicts a bar graph showing extracellular protease activity (AU) in non-irradiated and broad-beam irradiated cells (1 and 20 Gy), 5 minutes post irradiation (n=10).

FIG. 236 depicts a graph illustrating blood-brain barrier leakage assessed by combined 3/10 kDa fluorescent tracers after 20 Gy irradiation of mouse brain in vivo. Leakage was quantified relative to the non-irradiated hemisphere. ‘Non-irr’ is a non-irradiated control. Both an early (1 h) and late peak (48 h) of significant barrier leakage can be discerned (p<0.05; n=6). Experiments in C57BL/6 Cx43fl/fl:Tie2-Cre mice that have targeted Cx43 knockdown in endothelial and hematopoetic cells under control of the Tie2 promoter demonstrated significantly reduced barrier leakage (arrow pointing to the 1 h data point at the 100% control line; n=2).

FIG. 237 depicts an image showings the X-ray irradiation setup with CT scanner for in vivo brain irradiation.

FIG. 238 depicts show the 5×5 collimator used.

FIG. 239 depicts images illustrating different incidences used for 20 Gy irradiation of the left hemisphere only.

In the different figures, the same reference signs refer to the same or analogous elements.

### DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. The term “comprising” therefore covers the situation where only the stated features are present and the situation where these features and one or more other features are present. Thus, the scope of the expression “a product comprising means A and B” should not be interpreted as being limited to products consisting only of components A and B. It means that with respect to the present invention, the only relevant components of the product are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

The following terms are provided solely to aid in the understanding of the invention.

As used herein, and unless otherwise specified, a disorder or medical disorder is an abnormal physical or mental condition of a subject. ‘Disorder’ therefore encompasses the following terms: disease, illness, medical condition, pathology, syndrome, complication, sequela, etc. The disorder may in general be short or long-term and symptomatic or asymptomatic.

As used herein, and unless otherwise specified, a subject is a human or animal being. The subject may, for example, be a patient, e.g. in an ionizing radiation treatment (which may be completed, ongoing or to be performed), but may likewise be medical personnel (e.g. a doctor, nurse or operator).

As used herein, and unless otherwise specified, an agent or pharmaceutical agent is a substance or compound having a pharmaceutical effect. The agent may, for example, be an inorganic molecule, an organic molecule, a biomolecule (e.g. a peptide or protein) or a supramolecular assembly thereof; the agent may range in size from a small molecule to a macromolecule.

As used herein, and unless otherwise specified, when reference is made to a connexin hemichannel reference is made to a ‘free’ hemichannel which is not incorporated into a gap junction.

As used herein, and unless otherwise specified, when reference is made to a hemichannel or gap junction of a particular connexin isotype, it is meant that said hemichannel or gap junction at least comprises, and preferably consists of, the corresponding connexin proteins of that isotype. For example, a Cx43 hemichannel comprises at least one Cx43 protein and preferably consists of six Cx43 proteins; in other words, the Cx43 hemichannel may be a heteromeric or homomeric Cx43 hemichannel.

In a first aspect, the present invention relates to an agent for inhibiting a connexin protein, connexin hemichannel or connexin gap junction, for use in the therapy of one or more ionizing radiation-induced disorders.

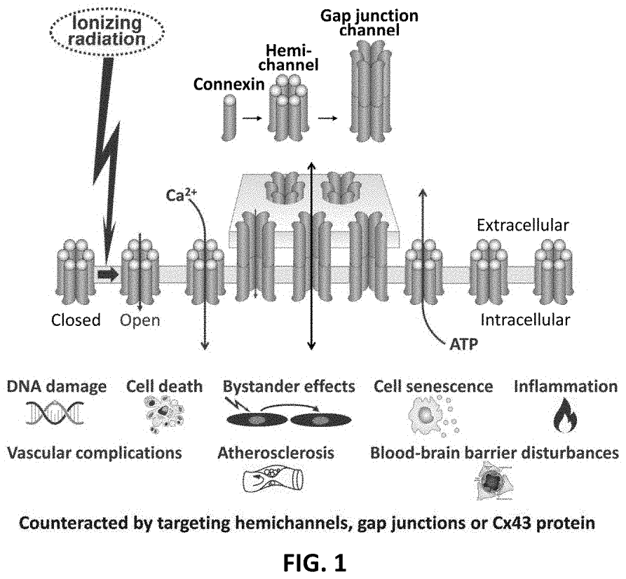

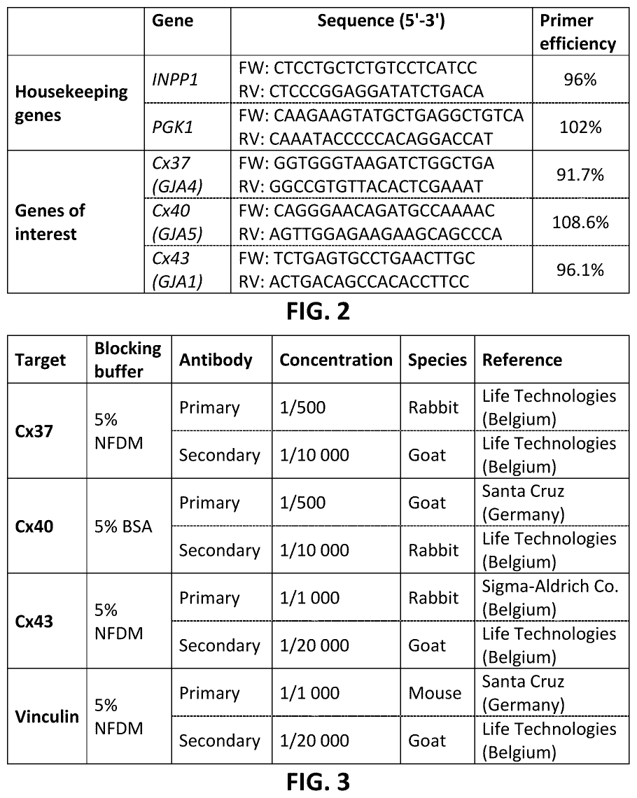

It was surprisingly realized within the present invention that connexins are involved in a variety of different ionizing radiation-induced secondary health effects. For example, the expression of Cx43, the most abundant connexin isotype in the human body, increases in response to ionizing radiation. Moreover, gap junctional cell-cell coupling as well as hemichannel opening increases, which collectively leads to disturbed cell-cell communication. An increase of gap junctional coupling enhances the propagation of cell death from dying cells to intact living cells by a mechanism of bystander cell death. Increased hemichannel opening results in ATP release that enhances inflammation, facilitates cellular Ca²⁺ overload, enhances cell death and leads to premature cell senescence. Combined, these effects act at the cellular as well as intercellular level, which may lead to ionizing radiation-induced disorders, including cardiovascular, neurovascular and neurodegenerative disorders. Crucially, counteracting hemichannel opening suppresses both cellular and intercellular levels of negative impact of radiation exposure. This is schematically depicted in FIG. 1. Further, it was surprisingly found that counteracting connexin hemichannel opening with hemichannel inhibitors, suppresses both cellular and intercellular pathways of negative radiation exposure impact and thereby function as radiation protective agents.

In general, the connexin may be of any isotype. There are 20 known connexins in mice and 21 in humans, which all form hemichannels as well as gap junctions. These distinct hemichannel isotypes can in principle all contribute, for example, to ATP release and calcium entry into the cells. It may then be expected that most, if not all, connexin isotypes hemichannels and/or gap junctions play a functional role in the cells and/or bodies where they are present. By functional role, it is here meant that the hemichannels and/or gap junctions can open and/or close under defined and realistic conditions for a particular cell type. Taking this notion into account, for example for connexin hemichannels, evidence for a functional role is presently available for several, but not yet all, connexins known. In particular, there is well-documented evidence available for a functional role of Cx43 and Cx26 hemichannels. For Cx30, Cx30.2, Cx32, Cx40 and Cx46 there is evidence, but less extensive than for Cx43 and Cx26. For Cx36, Cx37, Cx45, Cx45.6, Cx50 and Cx56 there is limited evidence available. For other connexins, the evidence is currently still sparse or absent. That said, the research on this topic is presently not concluded and it is reasonably believed that further additional evidence and functional roles for other connexins will still be found.

In preferred embodiments, the connexin protein may be a Cx43 protein, and/or the connexin hemichannel may be a Cx43 hemichannel, and/or the connexin gap junction may be a Cx43 gap junction. In other preferred embodiments, the connexin protein may be a Cx37, and/or the connexin hemichannel may be a Cx37 hemichannel, and/or the connexin gap junction may be a Cx37 gap junction. Cx43 (and to a lesser extent Cx37) proteins, hemichannels and gap junctions are particularly suitable targets, as described extensively in examples 1-3 (cf. infra). For Cx37, as set out in example 3, it had been expected that endothelial Cx43 KO in C57BL/6 Cx43fl/fl:Tie2-Cre mice would reduce bystander effects. However, because Cx37 displays compensatory increased expression, protection is masked suggesting that Cx37 also has bystander promoting effects and is therefore also an interesting target.

In embodiments, inhibiting a connexin protein may comprise inhibiting the expression of said connexin protein and/or inhibiting the formation of a connexin hemichannel or connexin gap junction therefrom. In embodiments, inhibiting a connexin hemichannel or connexin gap junction may comprise inhibiting the formation thereof and/or blocking or closing said hemichannel or said gap junction.

In embodiments, at least one of the one or more ionizing radiation-induced disorders may be a cardiovascular disorder, a neurovascular disorder, a neurodegenerative disorder or a dermal disorder. The disorder may, for example, be an inflammatory disease, cardiac ischemia/reperfusion, stroke, spinal cord injury, pain hypersensitivity, retinal disease, delayed wound healing, mental disorder (e.g. depression), etc. In preferred embodiments, the one or more radiation-induced disorders may be an ionizing radiation-induced atherosclerosis.

In embodiments, the therapy may comprise mitigation and/or prevention of one or more ionizing radiation-induced disorders. In embodiments, the radiation inducing the one or more disorders may be a medical intended radiation (e.g. as part of a radiation therapy), a medical non-intended radiation (e.g. a tissue surrounding a targeted area), an accidental radiation (e.g. due to a nuclear spill or accident) or an occupational radiation (e.g. as part of normal occupational handling). As such, e.g. in a radiotherapy, the one or more disorders may arise due to exposure of a targeted tissue to radiation or of an untargeted (e.g. surrounding) tissue in a patient, or exposure of an operator or assisting personnel. In embodiments, the therapy may be for reducing side-effects from a radiotherapy. In embodiments, the radiotherapy may be a thoracic radiotherapy or a head-and/or-neck radiotherapy (e.g. a brain radiotherapy).

In embodiments, the agent may be for inhibiting a first isotype (e.g. Cx43) of connexin protein, connexin hemichannel and/or connexin gap junction in a specific manner with respect to a second isotype (e.g. Cx37 or Cx40) of connexin protein, connexin hemichannel and/or connexin gap junction. Targeting specific connexin isotypes advantageously allows addressing selectively only those connexin isotypes which are involved in a particular disorder, thereby reducing the risk of side effects (e.g. which would occur when targeting other connexin isotypes which are not involved in the targeted disorder).

In embodiments, the agent may be for inhibiting one or more connexin hemichannels in a specific manner with respect to one or more connexin gap junctions (i.e. of the same or a different isotype). Gap junctions in particular fulfil important physiological functions and systemic administration of agents that inhibit gap junctions have considerable potential side effects, e.g. on cardiac pump function and heart rhythm. One, less preferred, strategy for reducing the above side effects may be to administer the agent locally (e.g. only to a targeted tissue). However, the risk of these side effects can be also advantageously be reduced significantly by targeting hemichannels specifically (i.e. with respect to gap junctions). This can then advantageously allow administering the agent in a broad manner (e.g. throughout the body). Moreover, in several cases, dysfunctional (e.g. leaky) hemichannels are the main cause of a particular disorder, more so than dysfunctional gap junctions. As such, it is then also for that reason advantageous to target hemichannels specifically.

In embodiments, the agent may be for inhibiting the connexin hemichannel in a specific manner with respect to a corresponding connexin gap junction (i.e. of the same isotype). For example, the agent may be for inhibiting Cx43 hemichannels specifically with respect to Cx43 gap junctions.

In embodiments, the agent may be a connexin-targeting molecule (e.g. a small molecule) or a hemichannel inhibitor. In embodiments, the agent may be a connexin mimetic peptide. In embodiments, the agent may be selected from carbenoxolone, a Gap19-based compound, a Gap26-based compound, an L2-based compound and a peptide5-based compound. Such a compound may, for example, be Gap19 (or Gap26, L2 or peptide5) as such, or may comprise or be derived from Gap19 (or Gap26, L2 or peptide5) fused to a membrane translocation sequence (e.g. TAT-Gap19). The membrane translocation sequence may advantageously facilitate cellular entry of the agent. Of the above, Gap19-based, L2-based and petide5-based compounds may be particularly preferred, as these can, for example, block Cx43 hemichannels in a specific manner. Gap junctions and hemichannels are typically composed of the same connexin building blocks; as a result, most substances that inhibit hemichannels also inhibit gap junctions. Nevertheless, agents have been identified which target hemichannels specifically with respect to gap junctions. For instance, peptides corresponding to certain sequences on the intracellular loop of a connexin (e.g. Cx43) have been found to specifically inhibit hemichannels while preserving gap junctional function. Examples of such peptides are Gap19 (a nonapeptide) and L2 (a 26 amino acid sequence that includes the Gap19 motif). L2 furthermore prevents gap junction closure, extending its potential effect spectrum to keeping Cx43 hemichannels closed and enhancing Cx43 gap junctional functions. Peptide5 is another interesting peptide in this respect, inhibiting hemichannels at low concentrations and hemichannels and gap junctions at higher concentrations. Peptide5 is currently believed to target not only Cx43 but also others connexin isotypes.

In embodiments, a dosage of the agent may be from 5 to 50 mg/kg, preferably between 10 and 40 mg/kg, yet more preferably between 15 and 35 mg/kg, most preferably between 20 and 30 mg/kg; such as 25 mg/kg. Such a relatively high dosage may be particularly relevant when the agent is a peptide (e.g. Gap19, such as TAT-Gap19), since the pharmaceutical activity of peptides is typically lower than for smaller molecules.

In embodiments, any feature of any embodiment of the first aspect may independently be as correspondingly described for any embodiment of any of the other aspects.

In a second aspect, the present invention relates to an agent for inhibiting a connexin protein, connexin hemichannel or connexin gap junction, for use in the therapy of an atherosclerosis.

It was surprisingly found within the present invention that connexins play a role in the pathology of atherosclerosis (both ionizing radiation-induced or not). Although healthy vascular endothelial cells mainly express Cx37 and Cx40, both connexins are lost in the endothelium covering advanced atherosclerotic plaques. In contrast, Cx43 typically has a low expression in the healthy endothelium, is found to increase the formation of atherosclerotic lesions in vivo, and becomes clearly detectable at specific regions of advanced atherosclerotic plaques. Without being bound by theory, it is believed that Cx37 and Cx40 act in an atheroprotective manner, while Cx43 has proatherogenic properties. As such, the agent may preferably be for inhibiting a Cx43 protein, Cx43 hemichannel or Cx43 gap junction.

In preferred embodiments, the agent may be for inhibiting a connexin hemichannel (e.g. Cx43 hemichannel).

In embodiments, any feature of any embodiment of the second aspect may independently be as correspondingly described for any embodiment of any of the other aspects.

In a third aspect, the present invention relates to a pharmaceutical composition comprising an agent according to any embodiment of the first or second aspect or a pharmaceutical composition.

In embodiments, the pharmaceutical composition may further comprise a pharmaceutically acceptable carrier.

In embodiments, any feature of any embodiment of the third aspect may independently be as correspondingly described for any embodiment of any of the other aspects.

In a fourth aspect, the present invention relates to a method of therapy of an ionizing radiation-induced disorder, comprising: (a) inhibiting a connexin protein, connexin hemichannel or connexin gap junction in a subject, and (b) exposing the subject to radiation; wherein step a is performed during and/or after step b.

‘Inhibiting’ is in this context understood to mean ‘bringing or keeping in an inhibited state’. In embodiments, step a may comprise administering an agent according to any embodiment of the first or second aspect or a pharmaceutical composition according to any embodiment of the third aspect. The agent or pharmaceutical composition may bring the connexin protein, hemichannel or gap junction in the inhibited state, where it may remain for some time (e.g. a few hours or days). The inhibition can be prolonged by subsequently administering additional amounts of the agent or pharmaceutical composition. Since ionizing radiation-induced disorders may typically develop in the days, weeks and/or months following the radiation exposure, the connexin protein, connexin hemichannel or connexin gap junction is advantageously inhibited during this time (i.e. after step b). As such, the agent and/or pharmaceutical composition may be administered before, during and/or after the radiation exposure, so that the inhibition may be in effect during and/or after said radiation exposure. In embodiments, administering of the agent and/or pharmaceutical composition may be repeated (e.g. during the days, weeks and/or months following the radiation exposure). This advantageously allows ensuring that inhibition remains into effect during the period where the ionizing radiation-induced disorder may develop.

In embodiments, step b may comprise directly exposing to radiation a cell comprising an inhibited connexin protein, connexin hemichannel or connexin gap junction, or exposing a surrounding cell thereof.

In embodiments, any feature of any embodiment of the fourth aspect may independently be as correspondingly described for any embodiment of any of the other aspects.

In a fifth aspect, the present invention relates to a use of an agent according to any embodiment of the first or second aspect in a method of therapy of an ionizing radiation-induced disorder.

In embodiments, the method of therapy may be a method of therapy according to any embodiment of the fourth aspect.

In embodiments, any feature of any embodiment of the fifth aspect may independently be as correspondingly described for any embodiment of any of the other aspects.

The invention will now be described by a detailed description of several embodiments of the invention. It is clear that other embodiments of the invention can be configured according to the knowledge of the person skilled in the art without departing from the true technical teaching of the invention, the invention being limited only by the terms of the appended claims.

(1): Single and Fractionated Ionizing Radiation Induce Alterations in Endothelial Connexin Expression and Channel Function

### Materials and Methods

### 1. Cell Culture

We used two human endothelial cell lines: hTERT telomerase immortalized human coronary artery endothelial cells (TICAE) from the European Collection of Authenticated Cell Cultures (ECACC; HCAECs Cat. No: 300-05), and telomerase immortalized human dermal microvascular endothelial cells (TIME) from the American Type Cell Culture (ATTC). TICAE cells are not tumorigenic and they display all major endothelial phenotypic markers, such as, PECAM1, vonWillebrand factor and cadherin-5. In addition, they have a response to radiation exposure similar to their primary counterparts. TIME cells are positive for CD31, capable of taking up Low Density Lipoprotein (LDL), and are karyotypically, morphologically, and phenotypically similar to the primary parent cells (data provided by ATTC).

TICAE and TIME cells were grown in MesoEndo Cell Growth Medium (Sigma-Aldrich Co. LCC, Diegem, Belgium). The passage number that was used in all the experiments (for controls and irradiated conditions) is between passages 26 until passage 32. The cells were kept in a humidified incubator at 37° C. supplemented with 5% CO2 and split every two to three days with a 0.05% trypsin solution supplemented with 0.02% ethylenediaminetetraacetic acid (EDTA) (Life Technologies, Merelbeke, Belgium). Cells were counted via Moxi Z Mini Automated Cell Counter (ORFLO Technologies, Ketchum, Id., USA). Cells were not sub-cultured during the course of the experiments, but medium was changed twice/thrice per week for 7 days and 14 days single irradiation experiments, and for 7 days fractionated irradiation experiment.

### 2. Irradiation

Both TICAE and TIME cells were irradiated at 100% confluence with a vertical point source X-ray beam using a Xstrahl RX generator (Camberley, UK; 320 kV, 12 mA, 3.8 mm Al and 1 mm Cu). X-rays doses (0.1, 0.5 and 5 Gy) were delivered to the cells either in one session (‘single irradiation’) or in a fractionated manner with three X-rays doses administered over three consecutive days (0.033 and 1.67 Gy/fraction/day), leading to an accumulative dose of 0.1 and 5 Gy (‘fractionated irradiation’). The dose rate used was 0.5 Gy/min for both single and fractionated exposure. Dosimetry was applied for all the experiments to ensure uniformity of dose and dose rate delivered, following ISO 4037 and ISO-17025 recommendations. Non-irradiated controls for all experiments were treated with the same conditions like irradiated samples, except they are sham-irradiated (0 Gy control).

### 3. Gene Expression Analysis Via RT-qPCR

TICAE and TIME cells were seeded in 6-well plates at a density of 2.5×10⁵ cells/well in five biological replicates. After three/four days, cells reached 100% confluence. The medium was changed before irradiation with 2 ml medium/well for single irradiation experiment, and 3 ml medium/well for fractionated irradiation experiment. Without refreshing the medium, cells were harvested 6, 24, 48 and 72 h post irradiation (p.i.). For the cells harvested 7 and 14 days p.i., the medium was refreshed every two/three days. After harvest, cells were stored in RLT Plus buffer and the total RNA was isolated with the RNeasy® Mini Kit (QIAGEN, Venlo, The Netherlands) according to the manufacturer's instructions. Concentration and quality of RNA were assessed spectrophotometrically using NanoDrop 2000c (Applied Biosystems, Thermofisher Scientific, Waltham, Ma, USA).

The GoScriprt™ Reverse Transcription System (Promega, Leiden, The Netherlands) was used to prepare the complementary DNA (cDNA) by adding 1 μL Random Nucleotide and 1 μL Oligo (dT) primers in 21 μL reactions. After denaturation, a mixture of 8 μL GoScriprt™ 5× Reaction Buffer, 6 μL magnesium chloride (MgCl₂), 2 μL PCR Nucleotide Mix, 1 μL Recombinant RNasin® Ribonuclease Inhibitor and 2 μL GoScript™ Reverse Transcriptase was added to each sample.

Two technical replicates for each biological replicate were prepared and quantitative polymerase chain reaction (qPCR) was performed in Fast Optical 96-Well Reaction Plates (Applied Biosystems, Gaasbeek, Belgium) using MESA GREEN qPCR MasterMix Plus for SYBR® Assay Low ROX (Eurogentec, Seraing, Belgium) on a 7500 Fast Real-Time PCR System (Applied Biosystems, Thermofisher Scientific, Waltham, Ma, USA). Amplification occurred at the following cycling conditions: 5 minutes 95° C., 40 cycles of 3 seconds at 95° C., and 45 seconds at 60° C., followed by the generation of a dissociation curve to verify amplification specificity. Reactions contained 12.5 μL 1× Fast SYBR Green Master Mix, 25 mM forward primer and 25 mM reverse primer (sequences selected from literature or qPrimerDepot database (https://primerdepot.nci.nih.gov/)), 20 ng of cDNA template (for all the conditions of all the experiments) and nuclease-free water in a total amount of 25 μL. To evaluate primer efficiency, a standard curve was generated using a two-fold dilution series of a sample over at least five dilution points. In addition, the specificity of the primers was confirmed by performing gel electrophoresis. Primer sequences and efficiencies are shown in Table 1. All measurements were performed in duplicate and the mean of two values from each sample was used in further analyses. The mathematical method of Pfaff1 was used to quantify the gene expression. Housekeeping genes, INPP1 and PGK1 were selected to obtain sample-specific normalization factors.

### 4. Protein Extraction and Western Blot Analysis

TICAE and TIME cells were seeded in 6-well plates at a density of 2.5×10⁵ cells/well in four to six biological replicates. After three/four days, cells reached 100% confluence. The medium was changed before irradiation with 2 ml medium/well for single irradiation experiment, and 3 ml medium/well for fractionated irradiation experiment. To extract proteins, 200 μL of RIPA lysis buffer (Roche, Brussels, Belgium), consisting out of 150 mM NaCl, 50 mM Tris-HCl pH 7.4, 1% NP-40/IGEPAL CA-630, 0.5% sodium deoxycholate, 0.1% SDS, a phosphatase tablet and protease inhibitor tablet (Roche, Brussels, Belgium), was added to 10⁶ cells. Next, cells were homogenized for 30 seconds with a tissue lyser II device (Qiagen, Antwerp, Belgium). The protein concentration was determined with the bicinchoninic acid (BCA) Protein Assay kit (Sigma-Aldrich Co. LLC, Diegem, Belgium). Subsequently, for all the conditions of all the experiments, 10 μg of proteins (except for Cx40 TIME, 20 μg was used) were supplemented with Laemmli buffer (¼ of the total volume) (Bio-Rad, Temse, Belgium), β-mercaptoethanol ( 1/10 of the Laemmli buffer) (Sigma-Aldrich Co. LLC, Diegem, Belgium) and heated at 95° C. for 5 minutes. Electrophoresis was performed at 160 volt and the separated proteins were transferred to a nitrocellulose membrane (Applied Biosystems, Thermofisher Scientific, Waltham, Ma, USA) using the iBlot dry transfer system (Invitrogen™ Thermo Fisher Scientific, Ninove, Belgium). The membranes were blocked for 2-3 h at room temperature using either 5% non-fat dry milk (NFDM) (Bio-Rad, Temse, Belgium) or 5% BSA (Sigma-Aldrich Co. LLC, Diegem, Belgium) (Table 2). Afterwards, membranes were incubated overnight at 4° C. with the appropriate primary antibody (Table 2). After washing, the membrane was incubated for 45 minutes at room temperature with the appropriate horse radish peroxidase (HRP)-conjugated secondary antibodies (Life Technologies, Merelbeke, Belgium) (Table 2). The HRP-immunoreactive bands were visualized with the ECL detection kit (Bio-Rad, Temse, Belgium) and scanned using the Fusion Fx imaging device (Vilber Lourmat, Eberhardzell, Germany). Signals were quantified densitometrically using Bio1D analysis software (Vilber Lourmat, Eberhardzell, Germany) and expressed as relative values (i.e. normalized to the corresponding vinculin signal of the same membrane). Cx37 protein level couldn't be detected with 10-30 μg protein concentration due to the low endogenous level.

### 5. Scrape Loading and Dye Transfer

The scrape loading and dye transfer assay (SLDT) was used to assess gap junctional function after IR exposure. TICAE and TIME cells were seeded in a 24-well plate at a density of 1×10⁵ cells/well in four to six biological replicates. Three days later, cells reached 100% confluence and the medium was refreshed before irradiation with 1 ml/well for single irradiation experiment and 1.5 ml for fractionated irradiation experiment. At different time points after irradiation (6 and 72 h post single irradiation, or 72 h post fractionated irradiation) the cells were washed 3 times with a scrape loading and dye transfer (SLDT) buffer (137 mM NaCl, 5.36 mM KCl, 0.81 mM MgCl₂, 5.55 mM MgCl₂.6H₂O, 25 mM HEPES, pH 7.4). Then, the cells were incubated with a SLDT solution composed of SLDT buffer and 400 μM 6-carboxyfluorescein (6-CF, 0.376 kDa) (Sigma-Aldrich Co. LCC, Diegem, Belgium) for one minute. A vertical scratch was made in the middle of each well, using a 20G needle (Becton Dickinson, Erembodegem, Belgium). After another one minute incubation, the cells were washed 6 times with HMS supplemented with 25 mM HEPES (Sigma-Aldrich Co. LCC, Diegem, Belgium). Carbenoxolone (Sigma-Aldrich Co. LCC, Diegem, Belgium; 50 μM) was used as a control for blocking gap junctional coupling. After an incubation time of 10 minutes, the cells were visualized with an Eclipse Ti automated inverted wide-field epifluorescence microscope (Nikon, Brussels, Belgium) equipped with a 5×dry objective (Plan Fluor, numerical aperture 0.6) and a Nikon IE2000-E camera controlled by the NIS Elements software. The relative area of 6-CF transfer was calculated using FIJI software. By analyzing the area of the dye diffusion from the first line of cells to adjacent cells, the gap junctional coupling was determined.

### 6. Extracellular ATP Measurements

Hemichannels are an ATP release pathway and we therefore determined extracellular ATP after irradiation. To that purpose, TICAE and TIME cells were seeded in a 96-well plate at a density of 1×10⁵ cells/well and 0.3×10⁵ cells/well, respectively, in six to eight biological replicates. Three days later, cells reached 100% confluence. At 30 minutes before irradiation, cells were refreshed with 100 μl medium alone or with medium supplemented with 100 μM TAT-Gap19 (Genosphere Biotechnologies, Paris, France) to block the Cx43 hemichannels. For fractionated irradiation, cells were refreshed with 150 μl medium/well. At different time points after irradiation (1, 6 and 72 h), 1:5 ATP assay mix dissolved in ATP mix dilution buffer (Sigma-Aldrich Co. LCC, Diegem, Belgium) was added to each well. Extracellular ATP release was assessed by measuring the luminescence signal received after oxidation of luciferin and ATP to oxyluciferin and AMP using the CLARIOstar® microplate reader (BMG Labtech, Temse, Belgium). Experiments were corrected for baseline ATP signal in medium in the absence or presence of TAT-Gap19.

### 7. Dye Uptake Assay

Hemichannel opening was also investigated by dye uptake studies making use of the hemichannel permeable fluorescent tracer propidium iodide (PI; MW 668.4 Da). In addition, dextran fluorescein 10 kDa dye was used to assess membrane integrity and occurrence of cell death. PI stains cells with open hemichannels, late apoptotic and necrotic cells, while dextran fluorescein only stains late apoptotic and necrotic cells. Therefore, PI-positive and dextran fluorescein-negative cells are a measure for hemichannel opening while dextran fluorescein-positive cells are a measure for dead cells.

TICAE and TIME cells were seeded in a 24-well plate at a density of 1.25×10⁵ and 1.5×10⁵ cells/well, respectively, in six biological replicates. Three days later, cells reached 100% confluence. Thirty minutes before irradiation, cells were refreshed with 1 ml medium alone or with medium supplemented with 100 μM TAT-Gap19 (Genosphere Biotechnologies, Paris, France). For fractionated irradiation, cells were refreshed with 1.5 ml medium/well. At different time points after irradiation (1, 6 and 72 h), cells were washed twice with HMS supplemented with 25 mM HEPES (Sigma-Aldrich Co. LCC, Diegem, Belgium) (HBSS-HEPES). Afterwards, the cells were incubated with 1 mM PI and 200 μM dextran fluorescein (10 kDa) (Life Technologies, Merelbeke, Belgium), dissolved in HBSS-HEPES for 5 minutes followed by 5 times washing with MSS-HEPES. Finally, the fluorescence was measured using an IncuCyte ZOOM® system (Essen BioScience, Ann Arbor, Mich., USA) by using different channels (TRITC, FITC and phase contrast) and a 10× objective Fluorescence signal was normalized to cell number.

### 8. Principal Component Analysis (PCA)

In order to have an overview of the results, principal component analysis (PCA) was performed for Cx40 and Cx43 gene expression and protein level (assessed by RT-qPCR and western blot) for both TICAE and TIME cells after single irradiation over the 6 h to 14 d p. i. time period. As the same sample was not used for both qPCR and western blot analysis, and the number of replicates were varying from 4-6 replicates, the means of the various replicates for each time point/radiation dose were calculated and PCA was performed using the prcomp command in R software (v 3.4.3). Both first and second components were plotted in the horizontal and vertical axes, respectively. For each time point and each radiation dose, the gene expression/protein level profile was averaged and analyzed using two-dimension PCA.

### 9. Statistical Analysis

All experiments were analyzed with a nonparametric Mann-Whitney T-test. The results were considered statistically significant when P<0.05. Data are presented as mean±standard error of the mean. Statistical analysis was done with GraphPad Prism 5.01 (GraphPad Software Inc., La Jolla, Calif. 92037 USA). Occasional exclusion of outlier data points were done using Grubbs' test.

### Results

### 1. Single and Fractionated Irradiation Induce Changes in the Gene Expression of Atheroprotective Cx37 and Cx40 and Proatherogenic Cx43

a. Single Irradiation

TICAE and TIME cells were exposed to different single doses of X-rays (0.1, 0.5 and 5 Gy) and assessed for changes in Cx gene expression at different time points (6 h, 24 h, 48 h, 72 h, 7 d and 14 d p.i.).

In TICAE cells, a radiation-induced downregulation in Cx37 gene expression was observed starting from 24 h p.i., which persisted up to 14 d p.i. and were mainly significant for the 0.5 Gy and 5 Gy doses. For 0.1 Gy exposures, Cx37 gene expression was significantly upregulated at an early time point (6 h p.i.), while downregulated at a late time point (7 d p.i.) (FIG. 4). In TIME cells, 5 Gy irradiation induced downregulation of Cx37 gene expression at 24 h and 14 d p.i., while upregulation was observed at 48 h. Irradiation at 0.1 and 0.5 Gy gave upregulation at 6 h and 48 h p.i. respectively (FIG. 5).

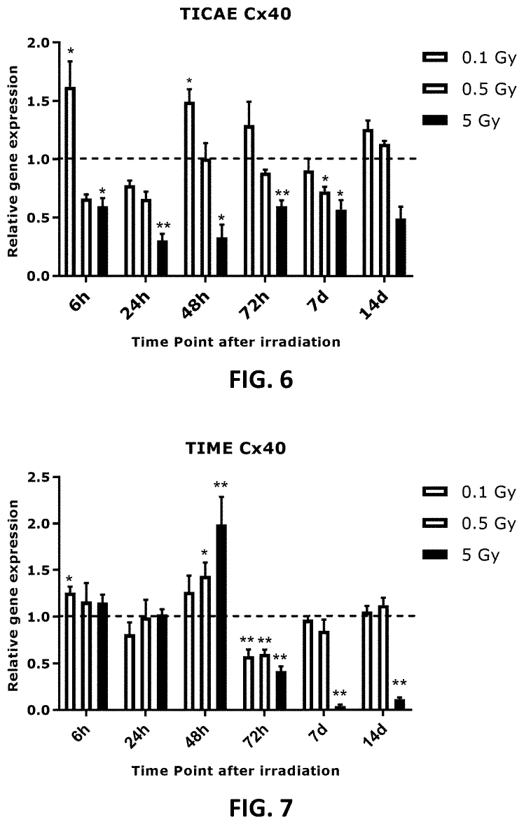

Gene expression of Cx40 was downregulated in TICAE cells starting from 6 h and persistent up to 7 d p.i., mainly significant at 5 Gy (FIG. 6). For 0.1 Gy exposures, Cx40 gene expression showed a significant upregulation at 6 h and 48 h p. i.. In TIME cells, Cx40 gene expression showed an upregulation at early time points, significant at 6 h for 0.1 Gy, and at 48 h for 0.5 Gy and 5 Gy. At 72 h p.i., a dose-dependent downregulation was observed, which persisted up to 7 and 14 d for the 5 Gy dose (FIG. 7).

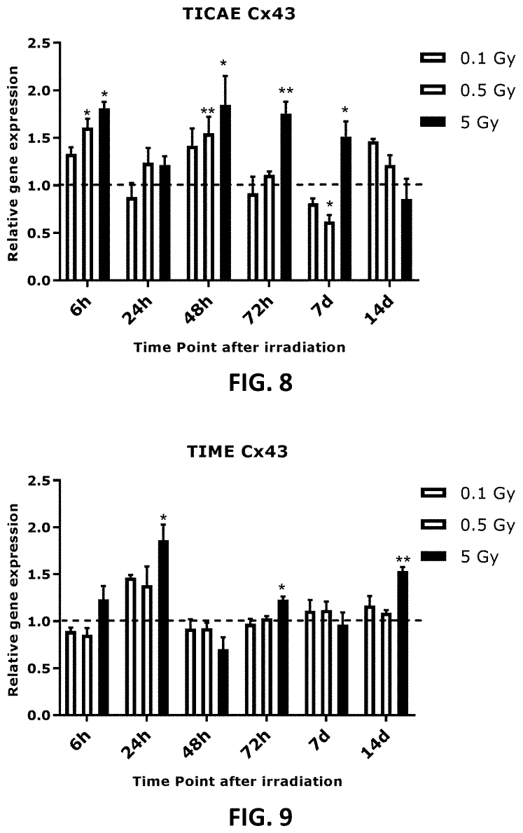

Different from Cx37 and Cx40, irradiation mainly resulted in an upregulation of Cx43 gene expression for the 0.5 and 5 Gy doses. In TICAE cells, 5 Gy induced significant upregulation of gene expression at 6 h and 48 h p.i., which persisted up to 7 d p.i.. For the 0.5 Gy dose, upregulation normalized more rapidly (FIG. 8). In TIME cells, 5 Gy caused significant upregulation at 24 h, 72 h and 14 d p.i., while lower doses did not induce significant alterations (FIG. 9).

a. Fractionated Irradiation

An early and late time point (24 h and 7 d p.i.) were selected to evaluate the effect of fractionated irradiation on Cx gene expression, and to compare it to single exposure effect (by comparing the relative gene expression (fold change) between single and fractionated exposure to their respective normalized controls).

For Cx37, fractionated irradiation of TICAE cells resulted in a downregulation of gene expression, which was significant for 5 Gy at both time points and for 0.1 Gy only at the 7 d time point, like single dose irradiation (FIG. 59 and FIG. 61). In line with the similar effect of both dose regimens, no significant differences were found between single and fractionated irradiation. In TIME cells, Cx37 gene expression was found to be significantly downregulated for the 5 Gy fractionated regimen at the 24 h time point only, as in single irradiation, but surprisingly the 7 d time point showed strongly significant upregulation. As expected from this strong upregulation, the response to fractionated 0.1 and 5 Gy irradiation was significantly higher as compared to single irradiation (FIG. 60 and FIG. 62).

For Cx40, fractionated irradiation of TICAE cells resulted in significant downregulation of gene expression for the 5 Gy dose at 24 h, like single irradiation, but not at 7 d, unlike single irradiation, resulting in significant differences for the two radiation regimens at 5 Gy for the 7 d time point (FIG. 63 and FIG. 65). In TIME cells, Cx40 gene expression was significantly downregulated at 5 Gy at the 24 h time point, and at 0.1 Gy and 5 Gy at the 7 d time point. In accordance, there is a significant difference between single and fractionated irradiation on Cx40 gene expression at 5 Gy at 24 h p.i. and at 0.1 Gy at 7 d p.i. (FIG. 64 and FIG. 66).

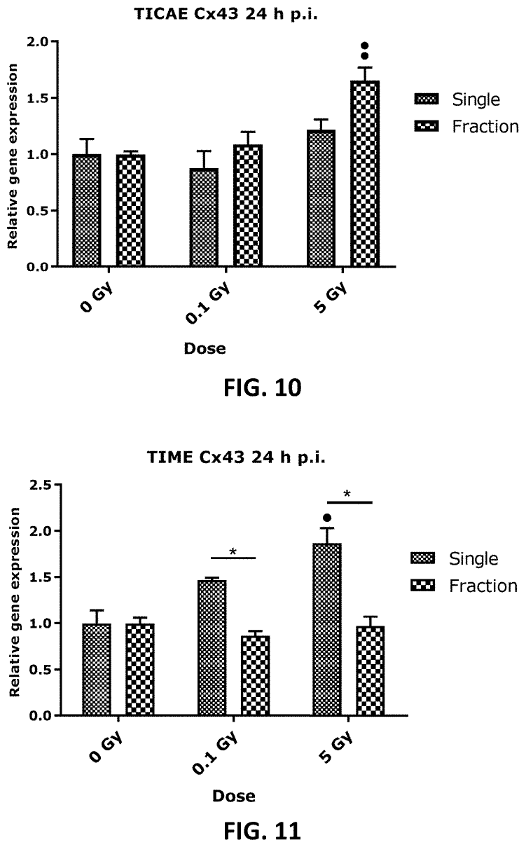

For Cx43, fractionated irradiation of TICAE cells induced significant upregulation of gene expression at the 24 h time point and things recovered at the 7 d time point, but there were no significant differences for the two radiation regimens (FIG. 10-FIG. 13). In TIME cells, fractionated irradiation did not induce changes in Cx43 gene expression after 24 h, unlike observations with single irradiation where 5 Gy caused upregulation. By contrast, at the 7 d time point, fractionated irradiation upregulated Cx43 gene expression (significant for both 0.1 and 5 Gy doses) while a single dose had no effect. As expected from the diverse effects of single vs fractionated irradiation, comparisons between the two dose delivery schemes showed several significant differences for the 24 h and 7 d time points as well as for the 0.1 and 5 Gy doses (FIG. 10-FIG. 13). Taken together with the observations for Cx37 and Cx40, these experiments clearly demonstrate distinct effects of fractionated irradiation vs single irradiation in TIME cells. For Cx40 and Cx43, the direction of the difference seems to depend on the time point after irradiation.

### 2. Single and Fractionated Irradiation Decrease Atheroprotective Cx40 Protein Expression and Increase Proatherogenic Cx43 Protein Expression

a. Single Irradiation

TICAE and TIME cells were exposed to different single doses of X-rays (0.1, 0.5 and 5 Gy) and assessed for changes in Cx protein levels at different time points (6 h, 24 h, 48 h, 72 h, 7 d and 14 d p.i.). Expression of Cx37 protein could not be detected with the 10-30 μg protein concentration used, indicating low endogenous levels.

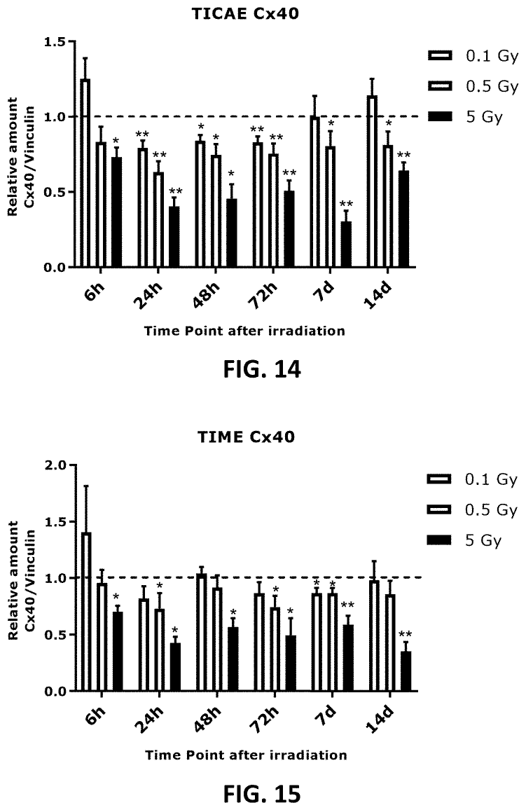

A radiation-induced acute and persistent decrease in Cx40 protein level was observed in a dose-dependent manner in both TICAE and TIME cells. In TICAE cells FIG. 14), only the 5 Gy dose demonstrated significantly decreased Cx40 expression at the early time point of 6 h. However, in the period between 24 h and 72 h all doses showed significantly decreased Cx40 protein levels; this decrease was continued up to 14 d p.i. for the 0.5 and 5 Gy doses. In TIME cells, the decrease in Cx40 protein level was significant at all time points for 5 Gy; for 0.5 Gy significant decrease was attained at 24 h, 72 h and 7 d p.i., while for 0.1 Gy significance was only attained at 7 d p.i. (FIG. 15).

We found a radiation-induced acute and persistent increase in Cx43 protein level that was dose-dependent for both TICAE and TIME cells. For the 5 Gy dose, the increase was significant for all time points except for 14 d p.i.; for the 0.5 Gy dose, all time points except 48 h were significant; for 0.1 Gy, a significant increase was apparent from 72 h on, which remained high up to 14 d p.i. (FIG. 18). In TIME cells, significant increases in Cx43 protein levels were mainly concentrated in the 24 h to 72 h time window, and at 7 d p.i., a slight, but significant decrease in Cx43 protein level was observed (FIG. 19).

In addition, a radiation-induced increase in the phosphorylated (p) and hyperphosphorylated (pp) forms of Cx43 were observed in TICAE cells at 24 h, 48 h, 7 d and 14 d p.i. (mainly significant at 0.5 Gy and 5 Gy) (FIG. 22). In TIME cells, a dose-dependent increase in the phosphorylated and hyperphosphorylated forms was observed mainly at 24 h p.i. (FIG. 27).

b. Fractionated Irradiation

An early and late time point (24 h and 7 d p.i.) were selected to evaluate the effect of fractionated irradiation on Cx protein expression, and to compare it to single exposure effect.

For Cx40, fractionated irradiation of TICAE cells induced a significant decrease in the protein level only for the 5 Gy dose at the 24 h and 7 d time points, but the effects were less pronounced compared to single irradiation at 7 d time points (FIG. 67 and FIG. 69). In TIME cells, fractionated irradiation induces a significant decrease at 5 Gy in Cx40 protein level at 24 h time point, like single irradiation, and then stabilizes at 7 d post fractionated irradiation, unlike single irradiation. In accordance, there is a significant difference between single and fractionated irradiation on Cx40 protein level at 5 Gy after 7 d of the exposure (FIG. 68 and FIG. 70).

For Cx43, fractionated irradiation of TICAE cells induced an increase in the protein level, which was most clear at the 24 h time point (significant for both 0.1 and 5 Gy doses) and less prominent at the 7 d time point (significant for 5 Gy only) (FIG. 24 and FIG. 26). In TIME cells, fractionated irradiation induced an increase in Cx43 protein level at 5 Gy for both 24 h and 7 d time point, however, these effects were less pronounced than those observed in TICAE cells (FIG. 25 and FIG. 27). Importantly, no significant differences were observed between single and fractionated irradiation in both TICAE and TIME cells.

### 3. Single and Fractionated Irradiation Exposure Induce an Increase in Gap Junction-Mediated Dye Coupling

a. Single Irradiation

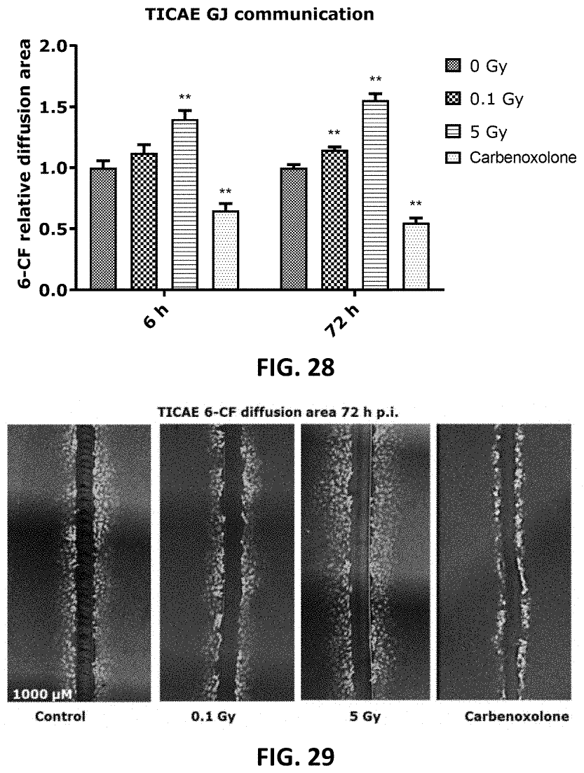

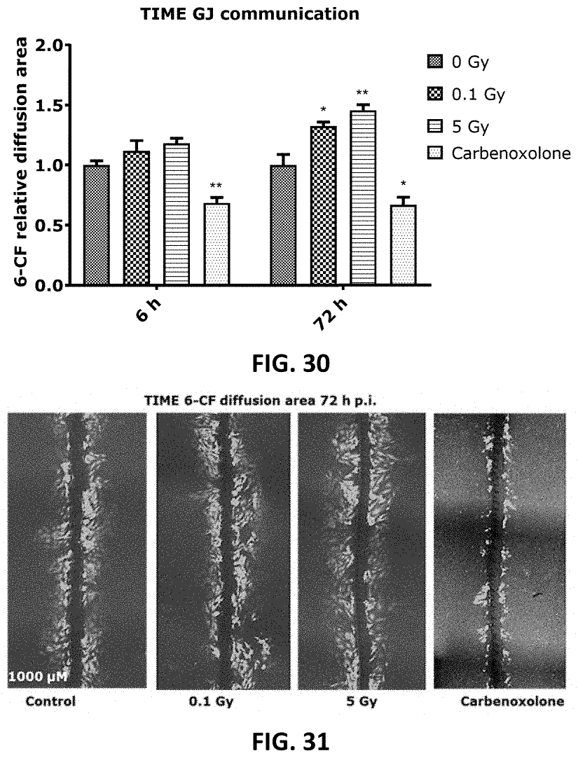

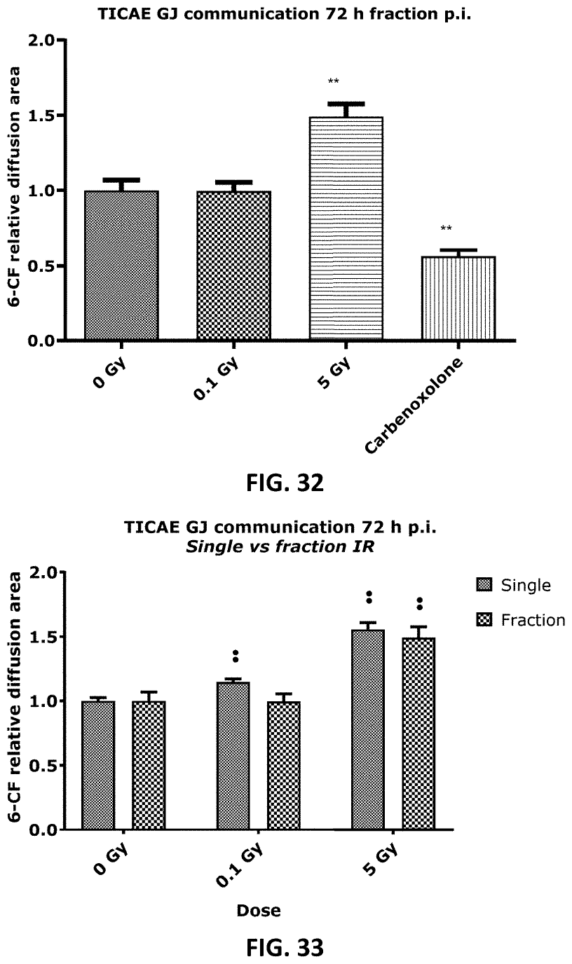

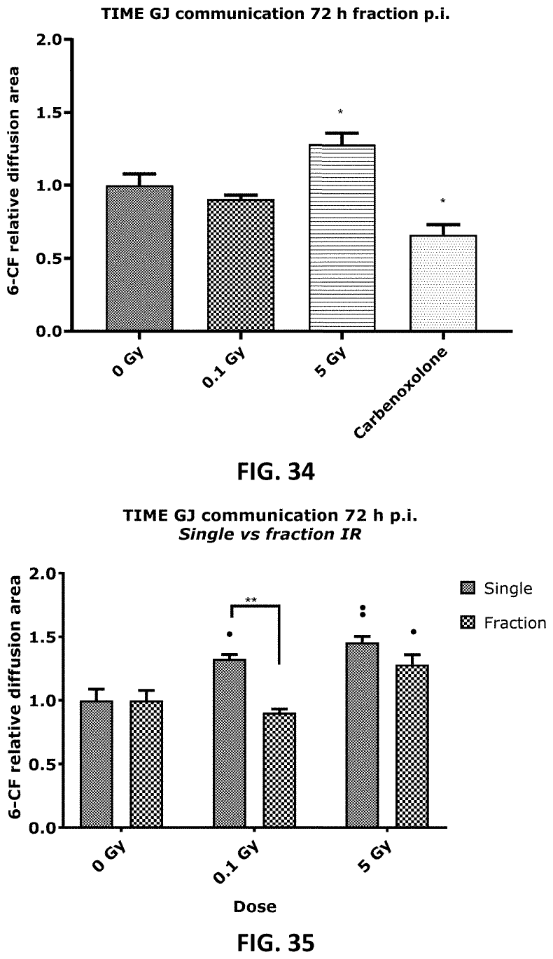

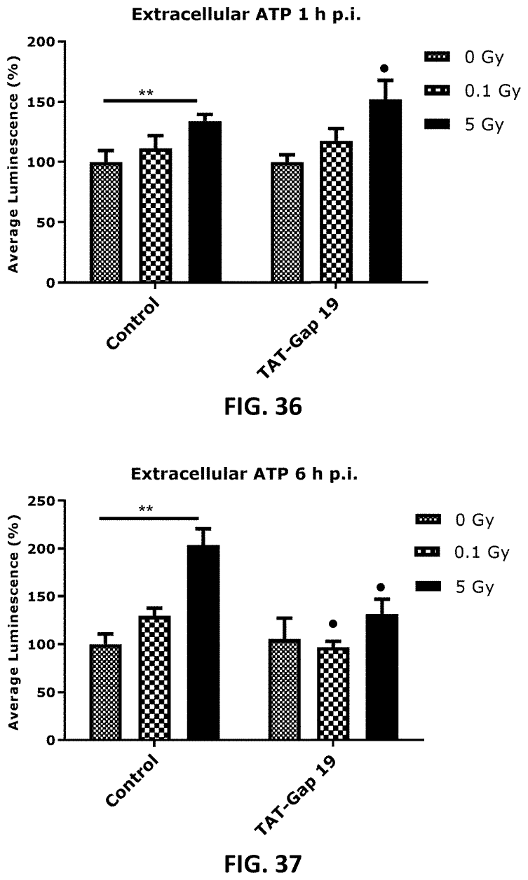

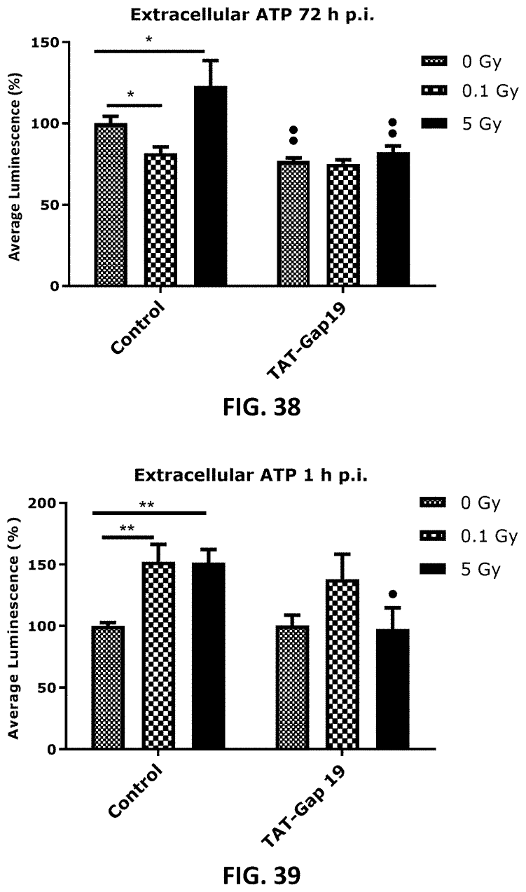

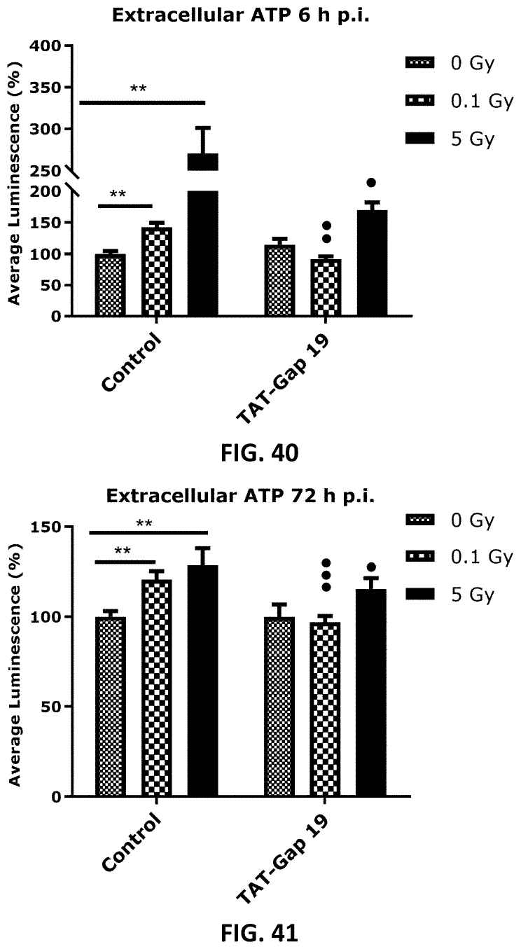

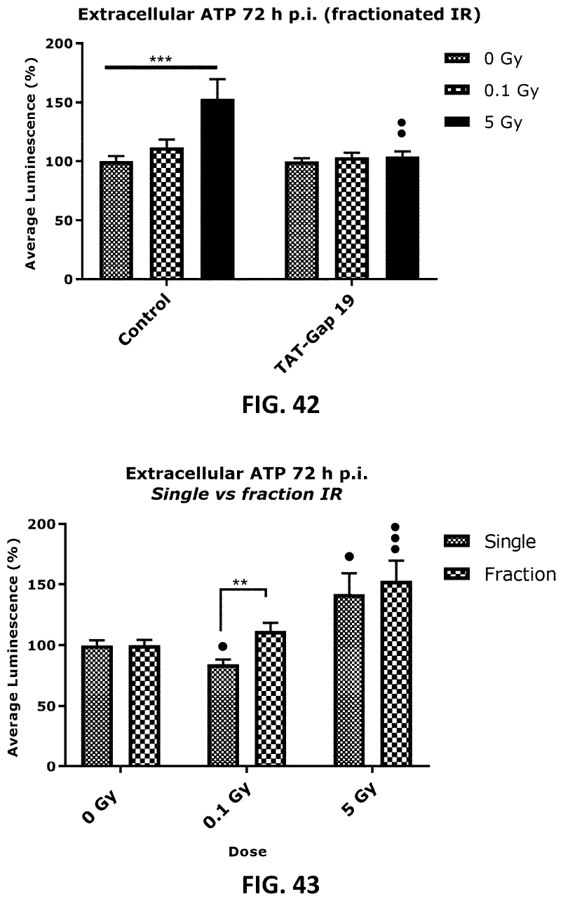

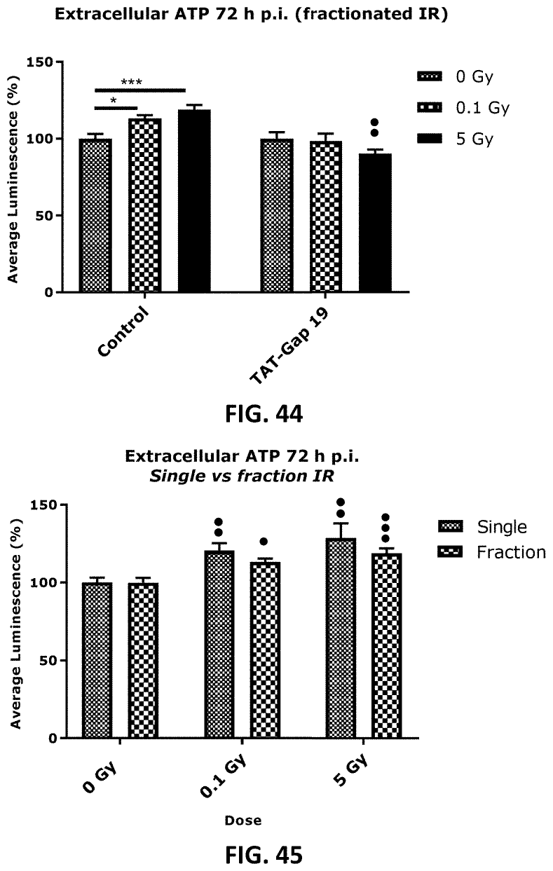

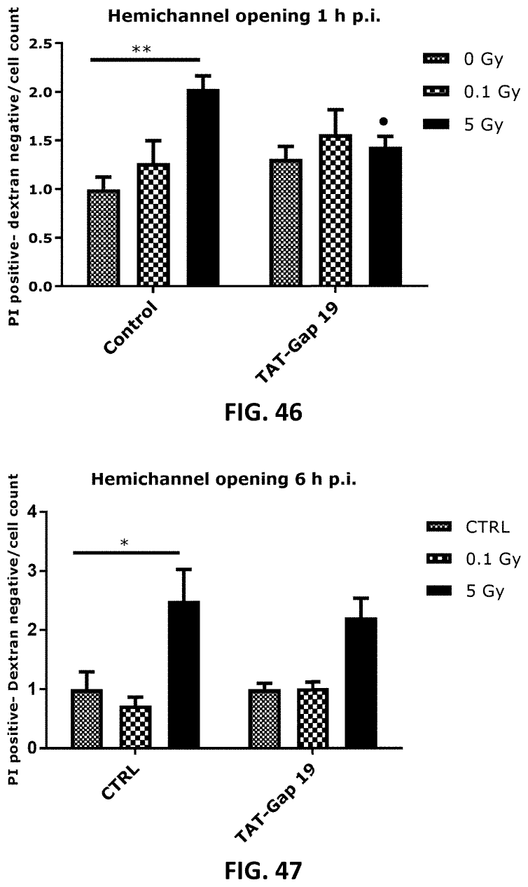

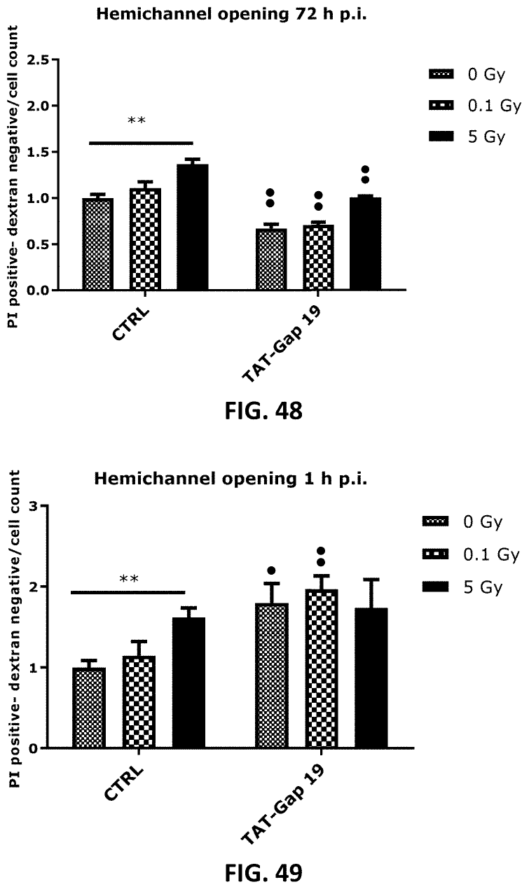

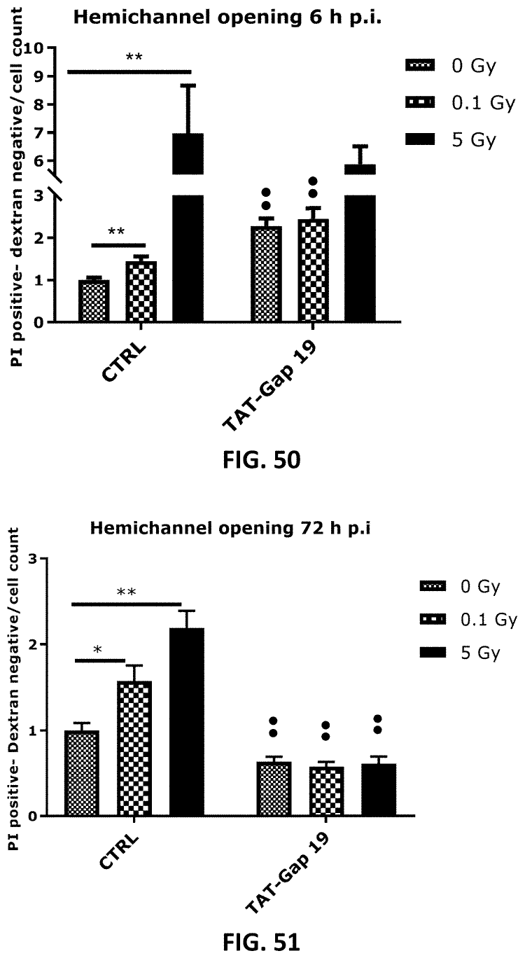

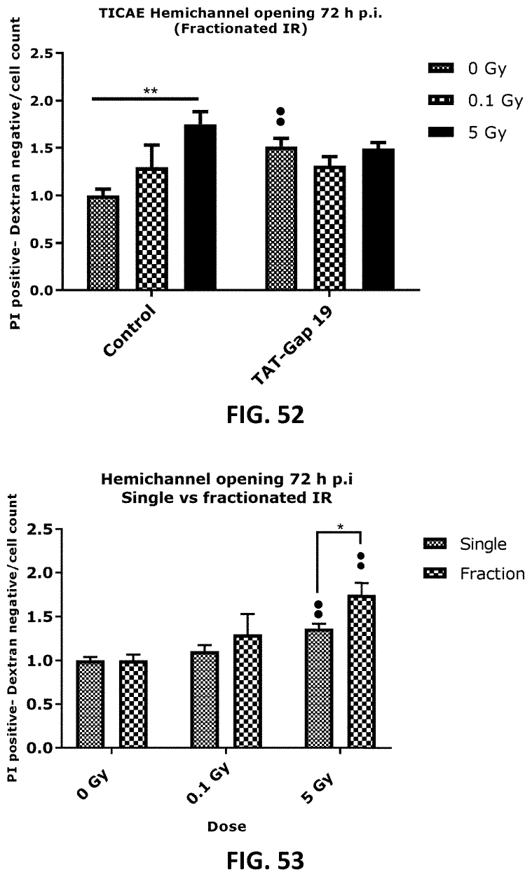

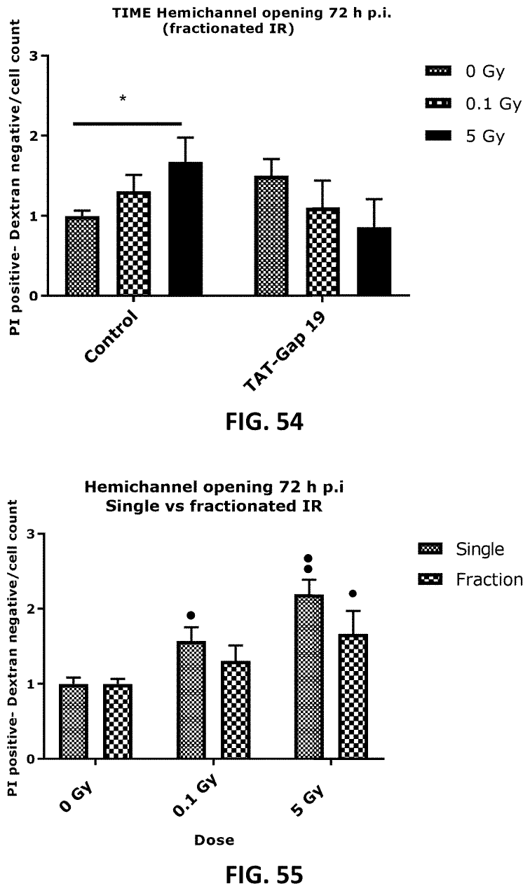

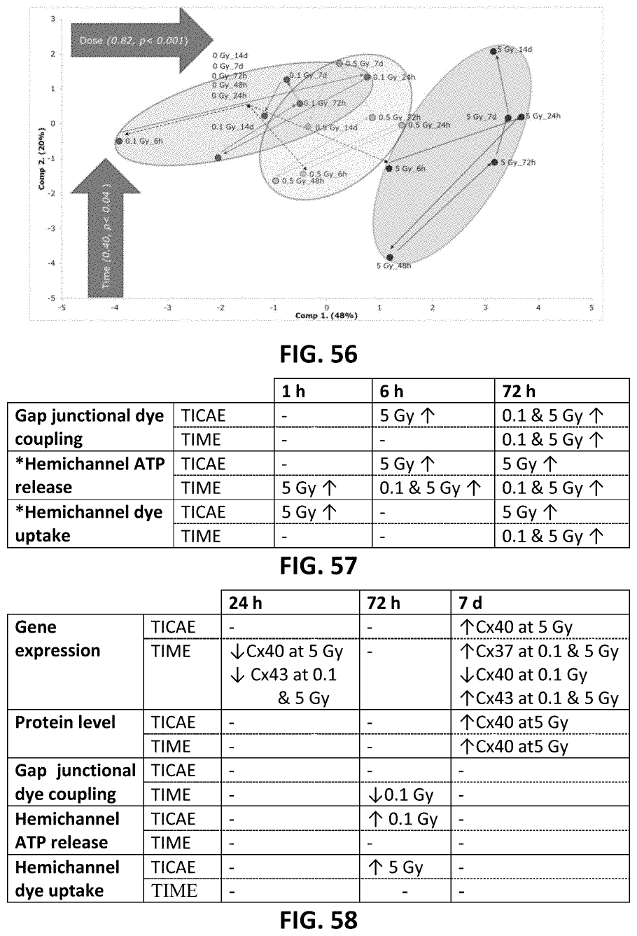

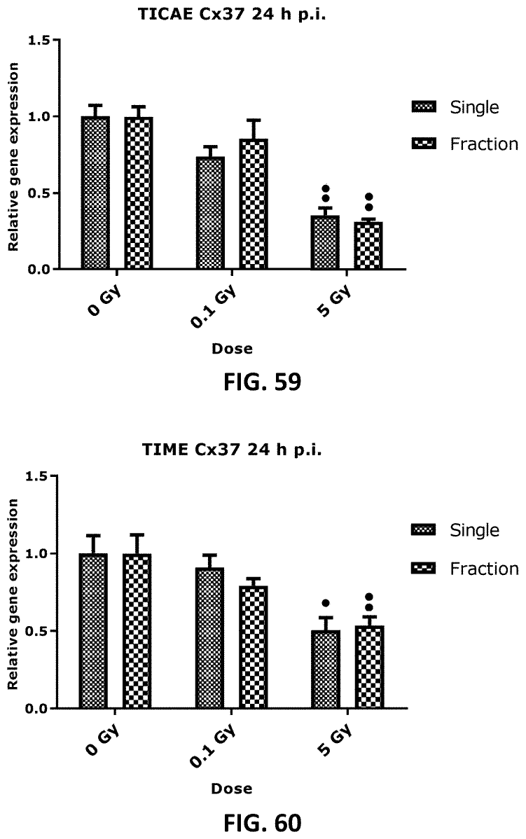

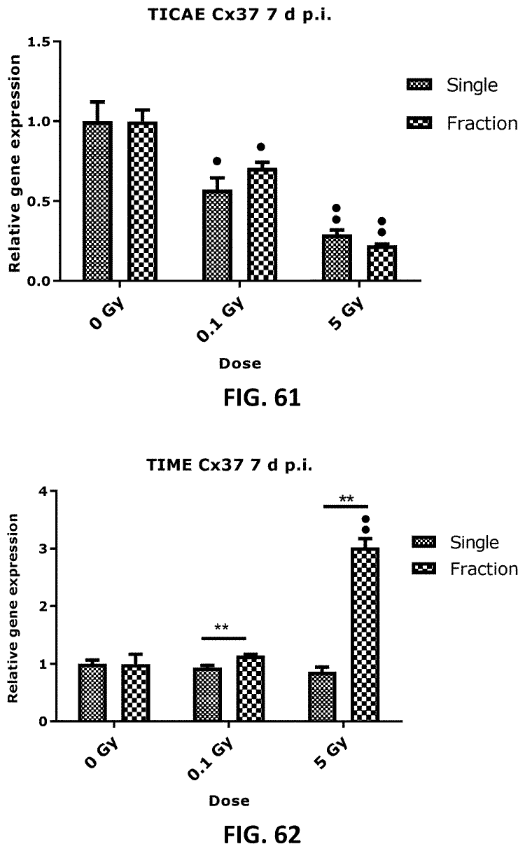

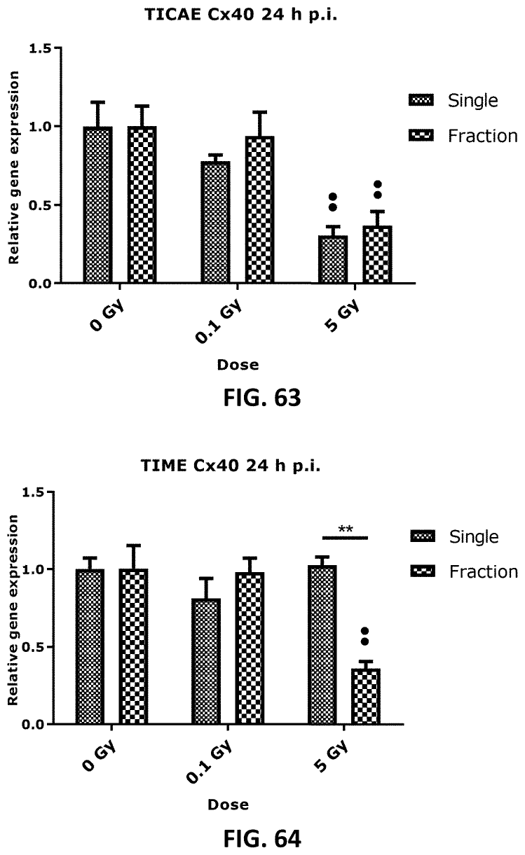

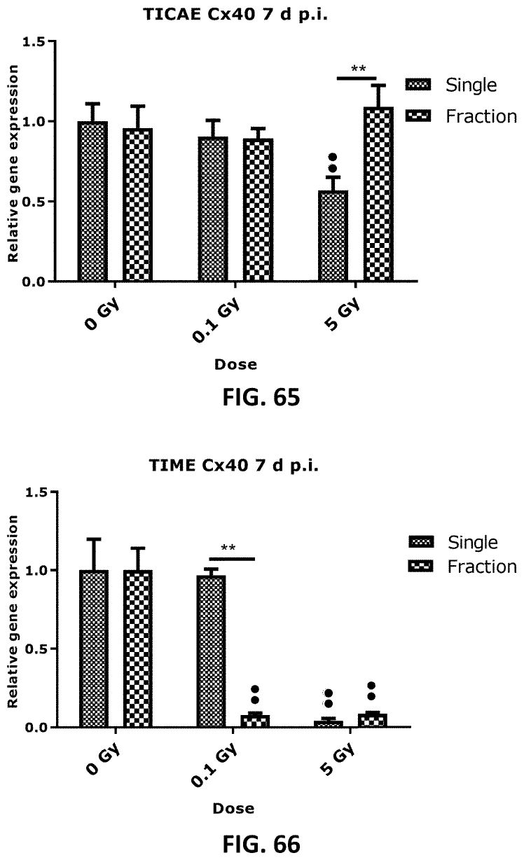

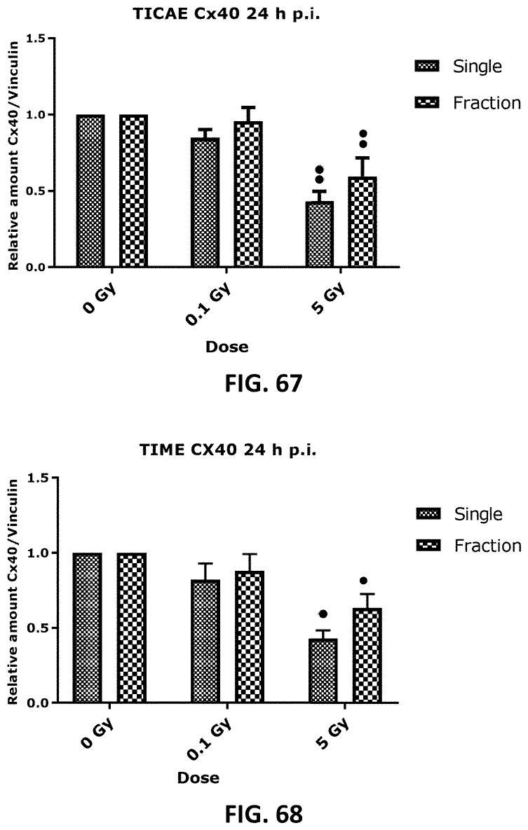

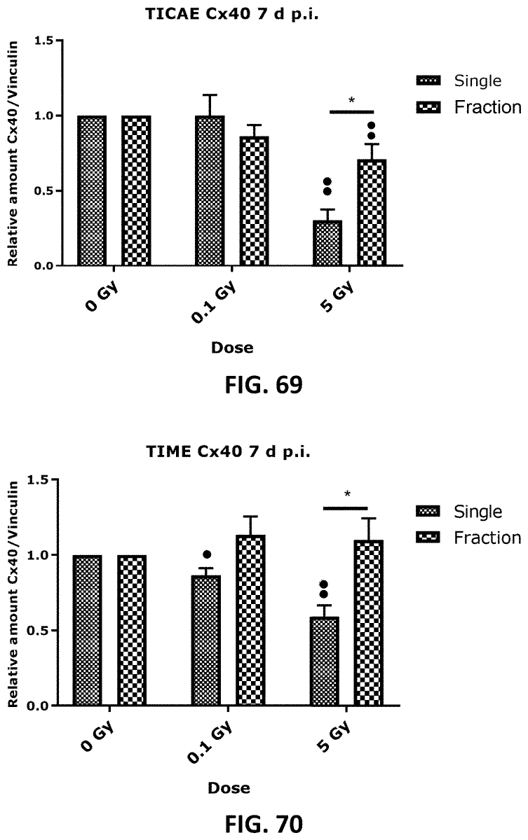

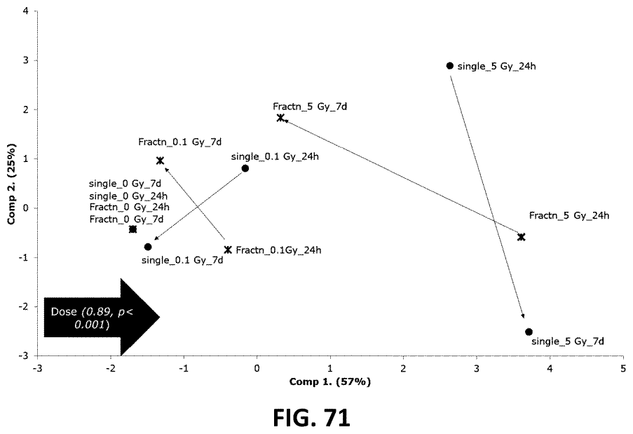

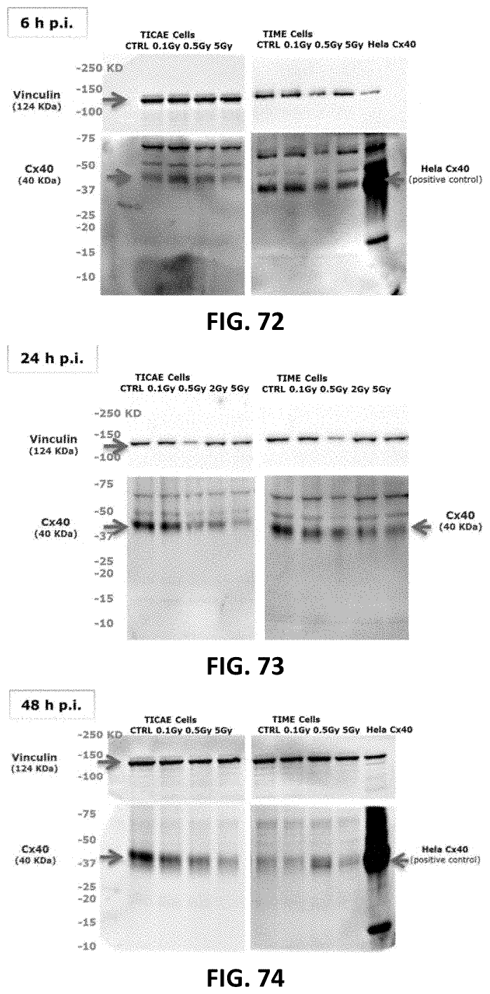

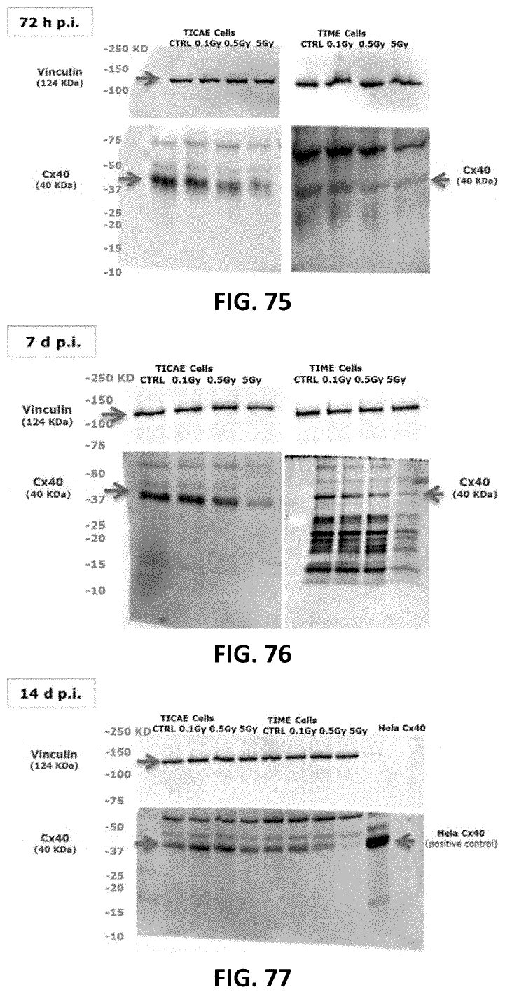

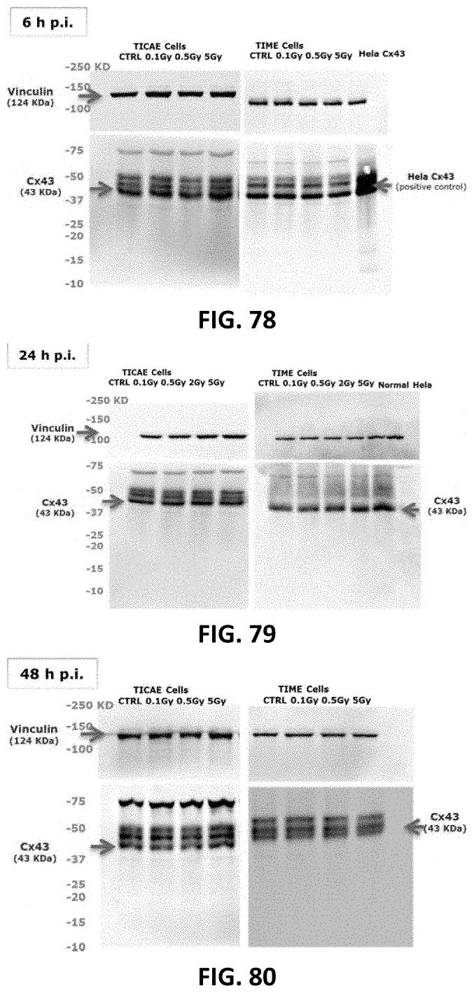

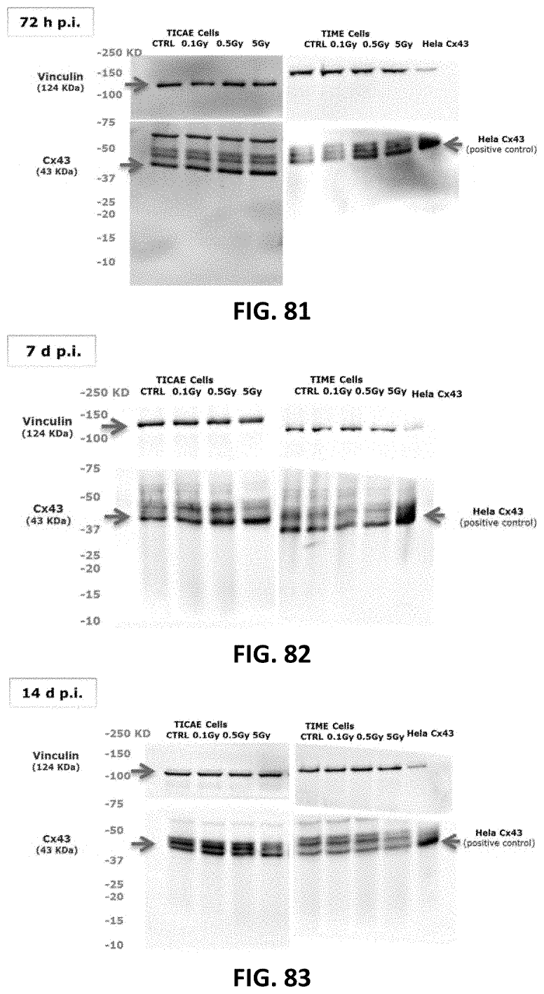

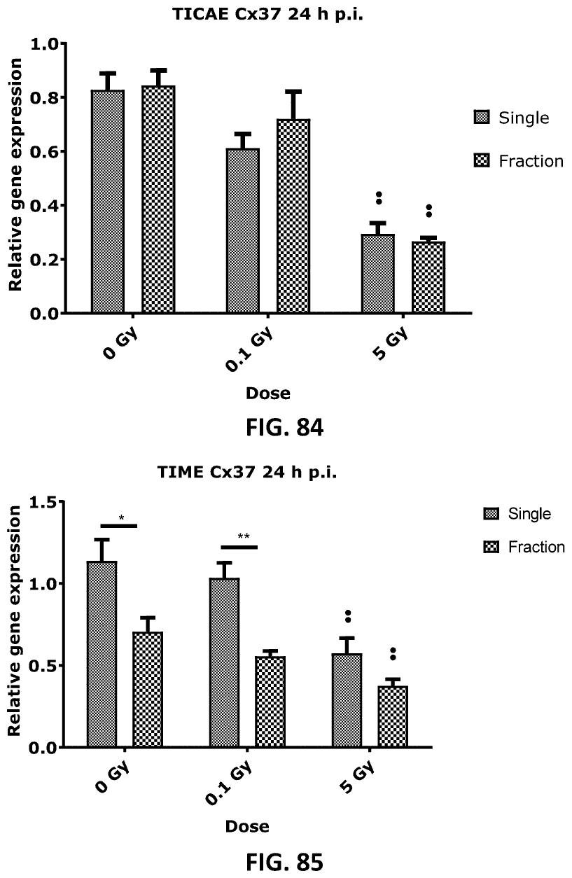

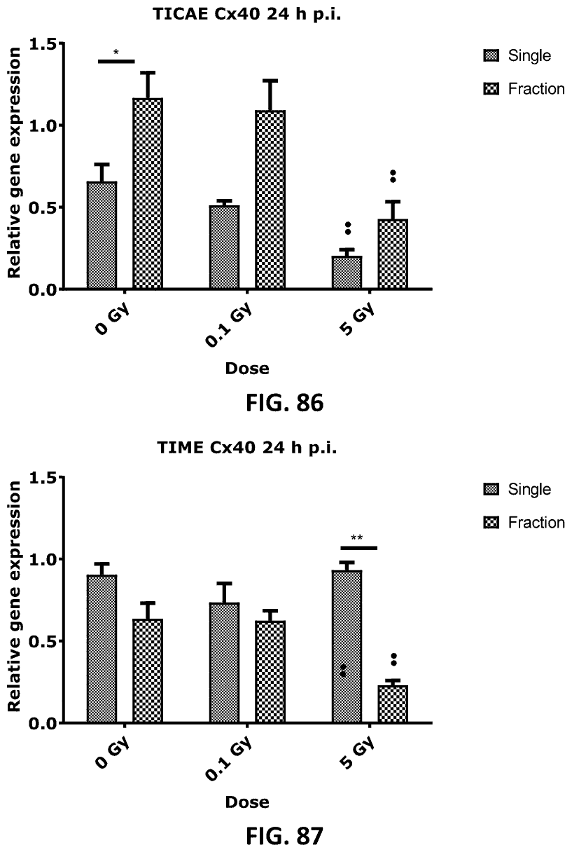

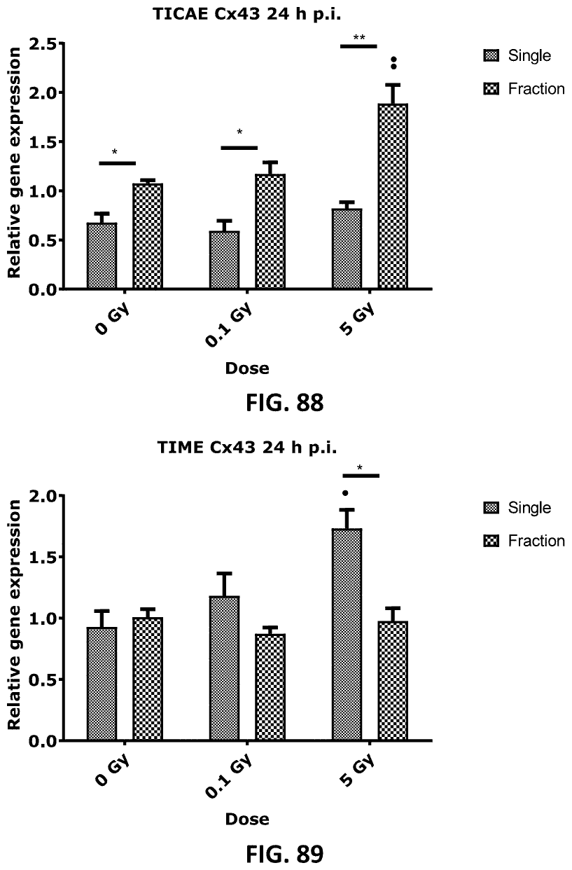

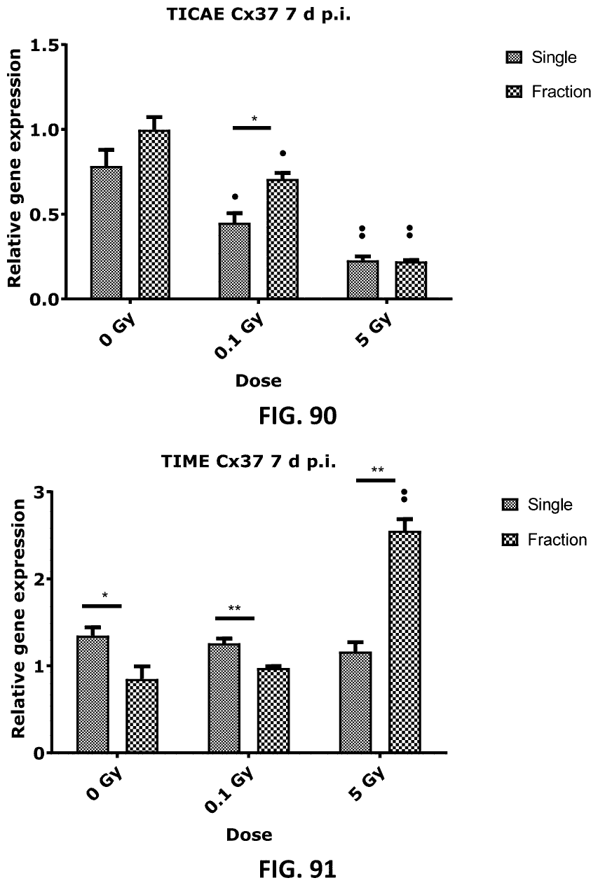

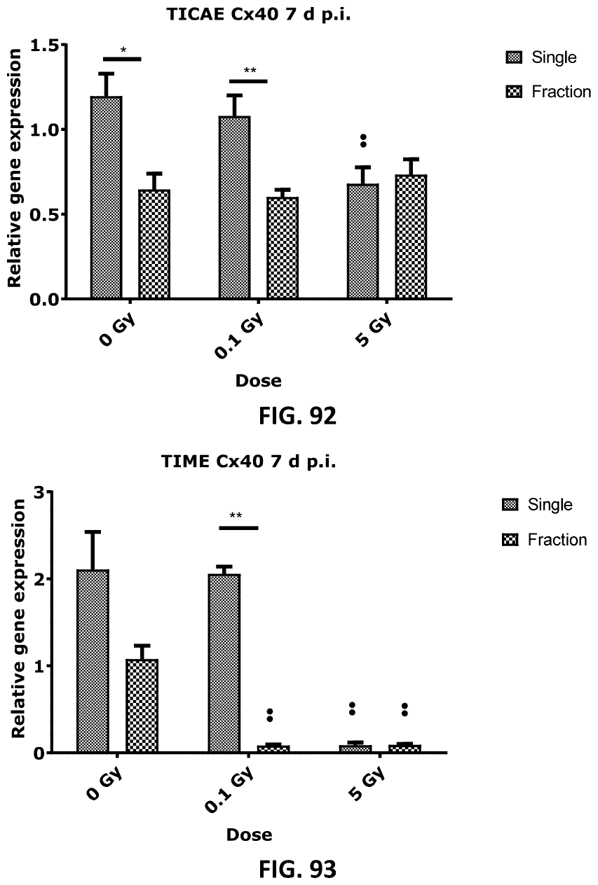

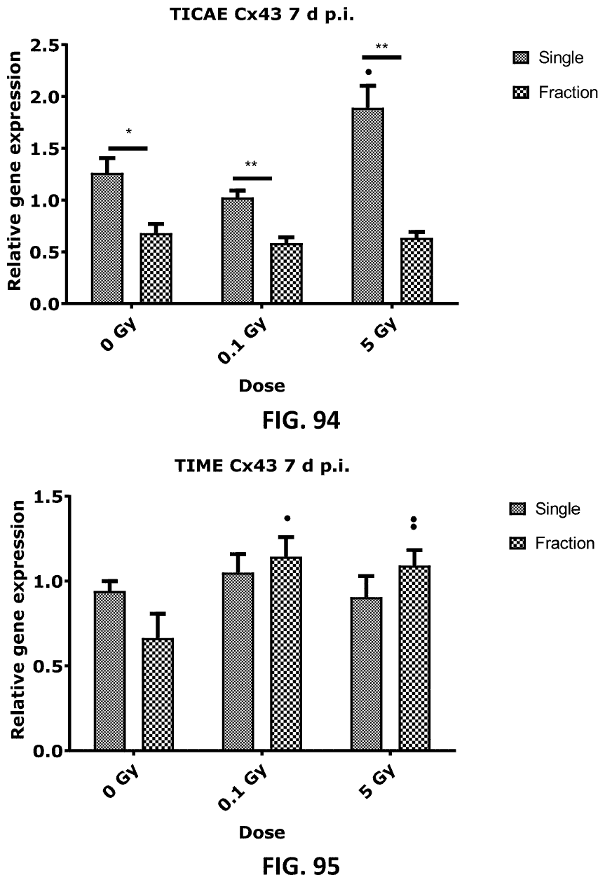

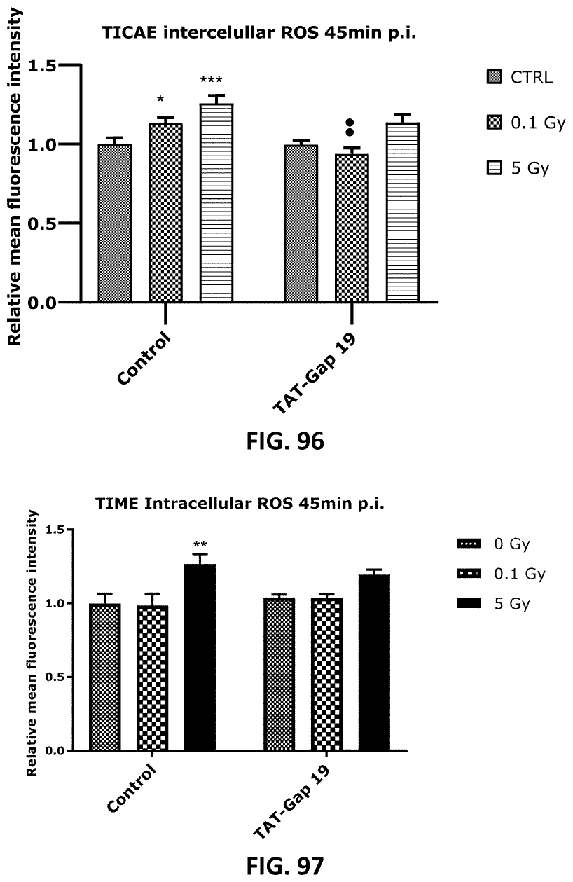

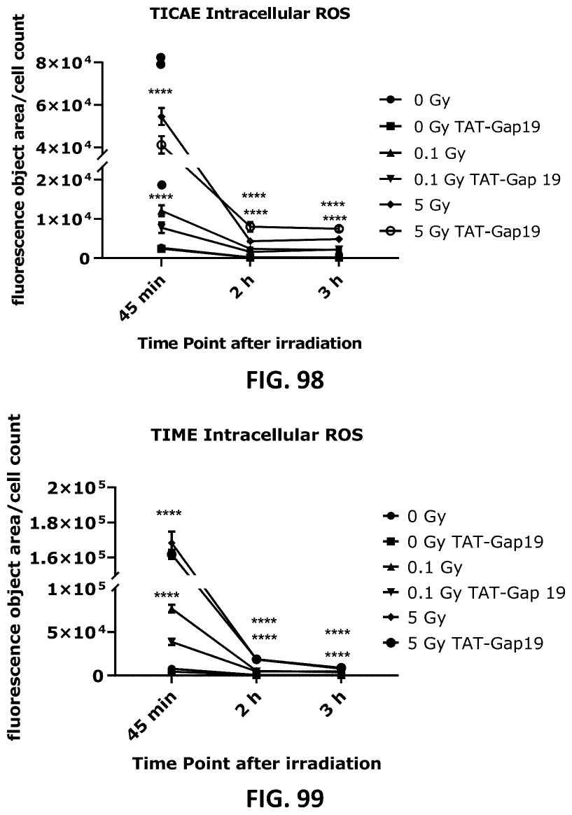

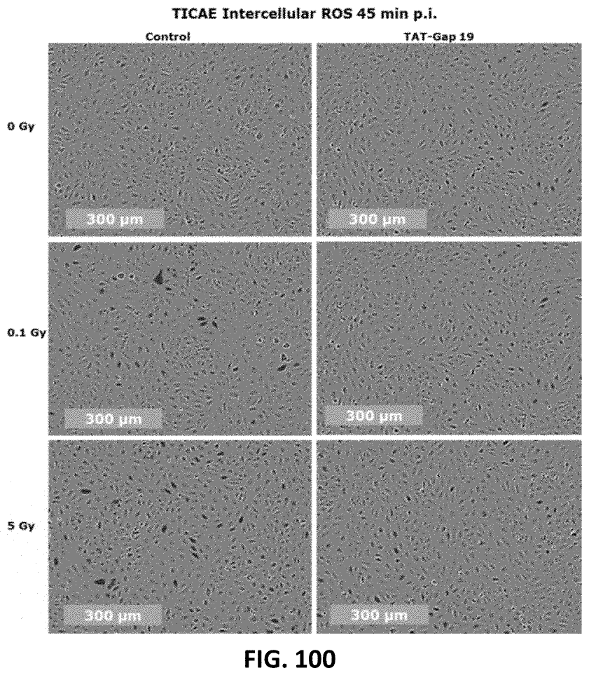

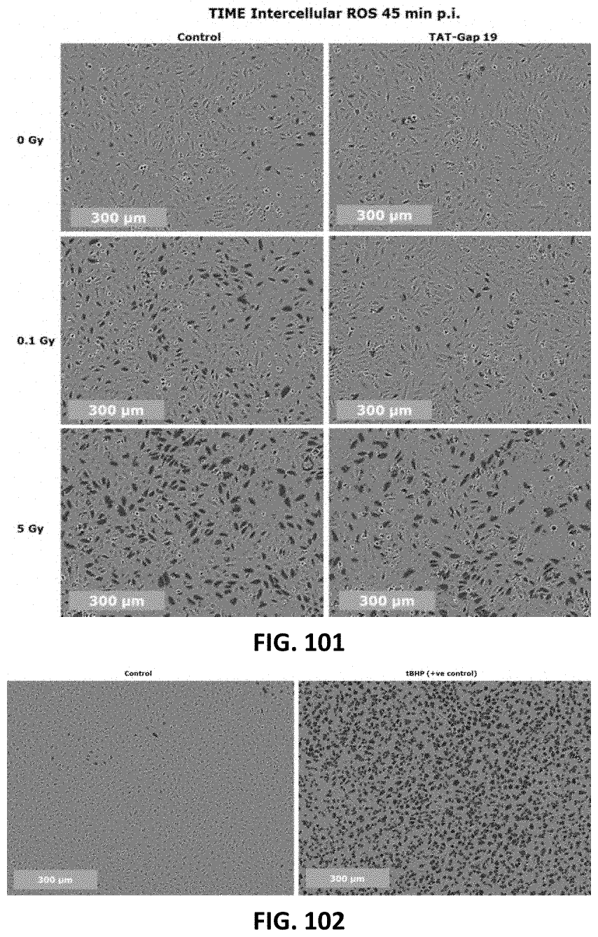

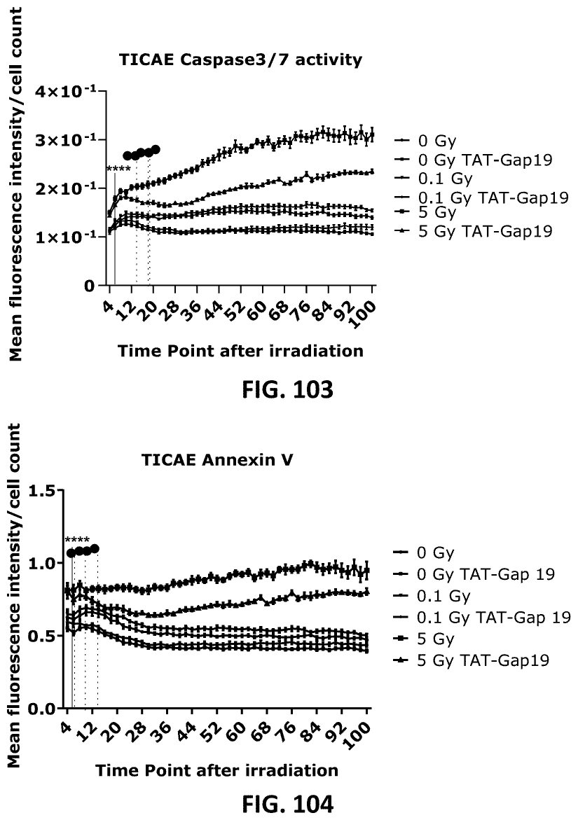

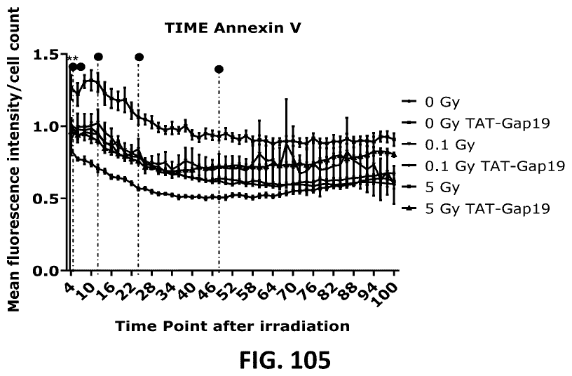

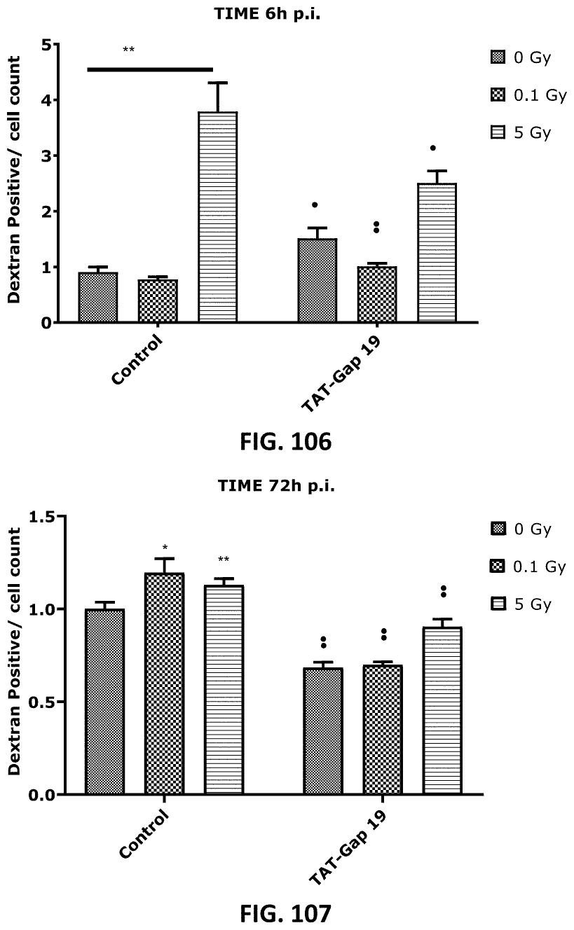

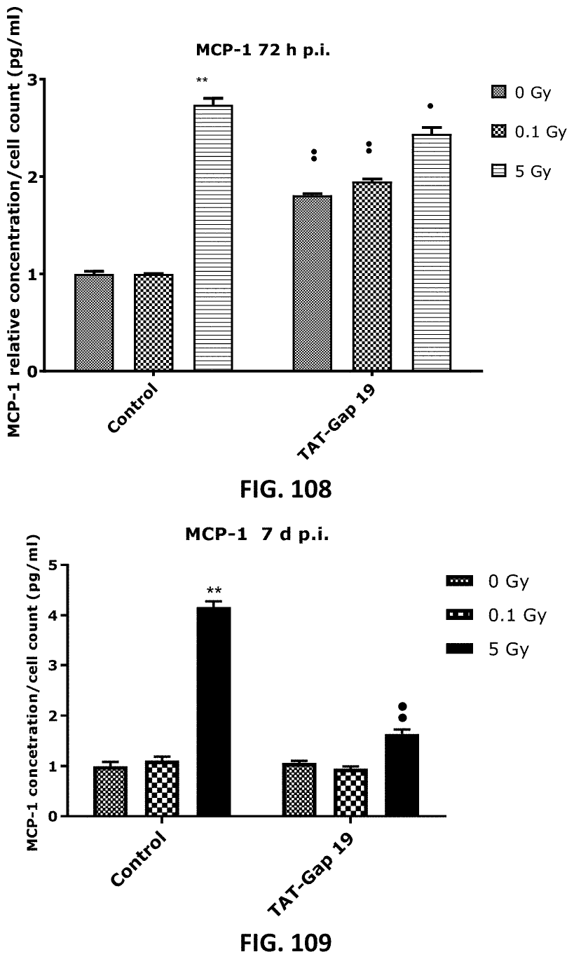

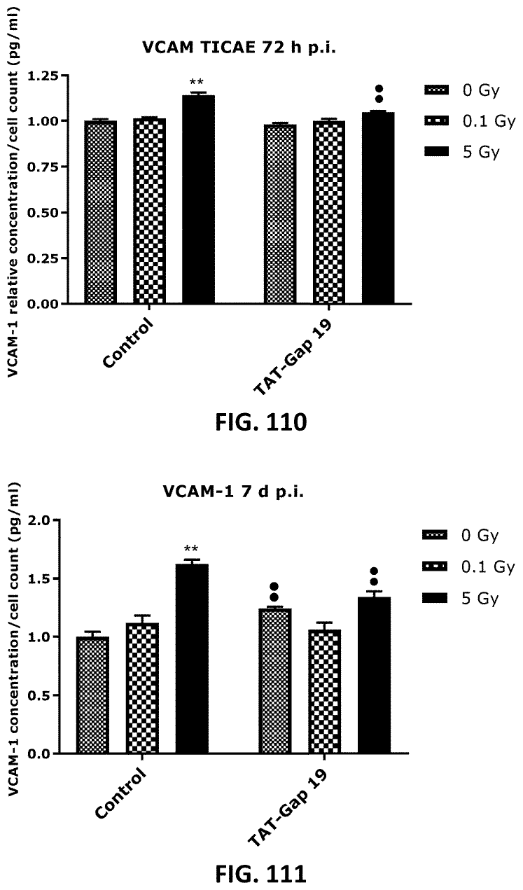

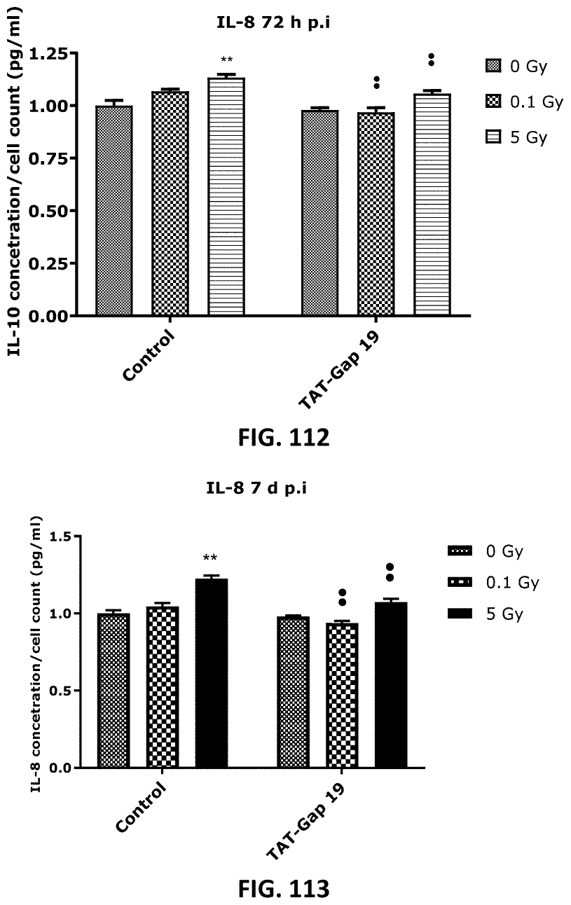

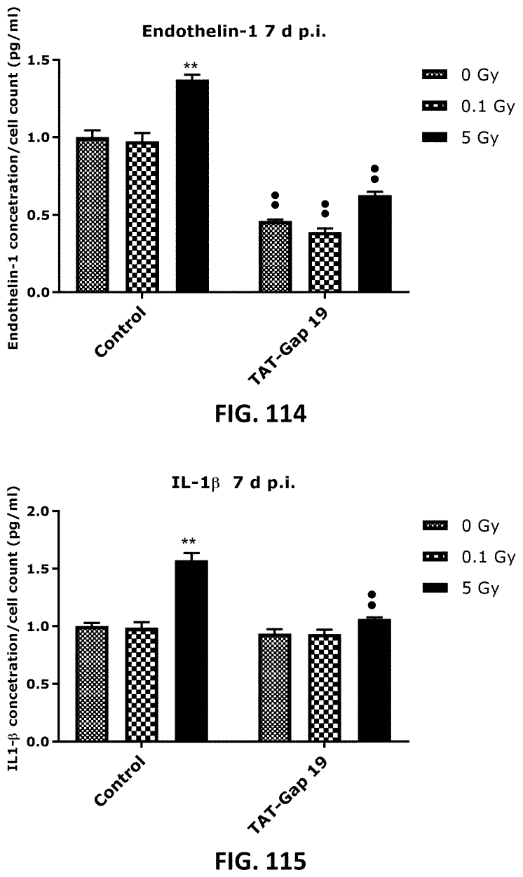

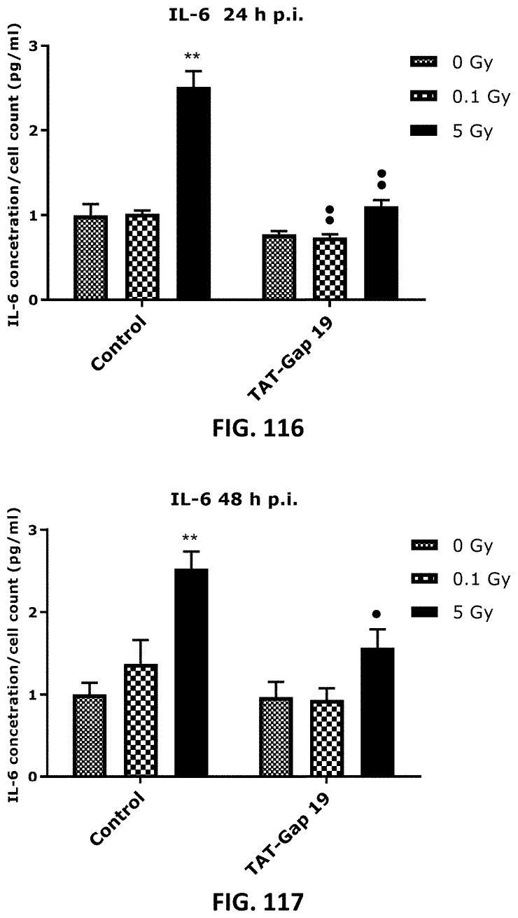

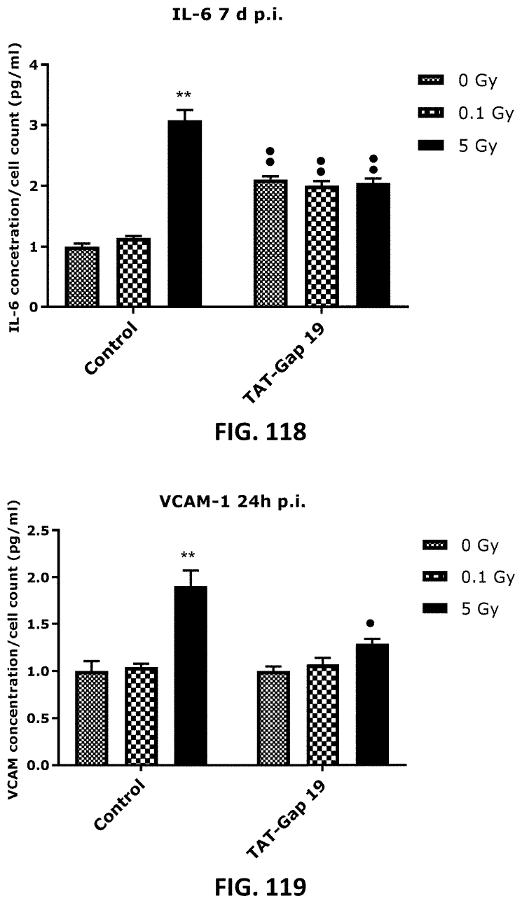

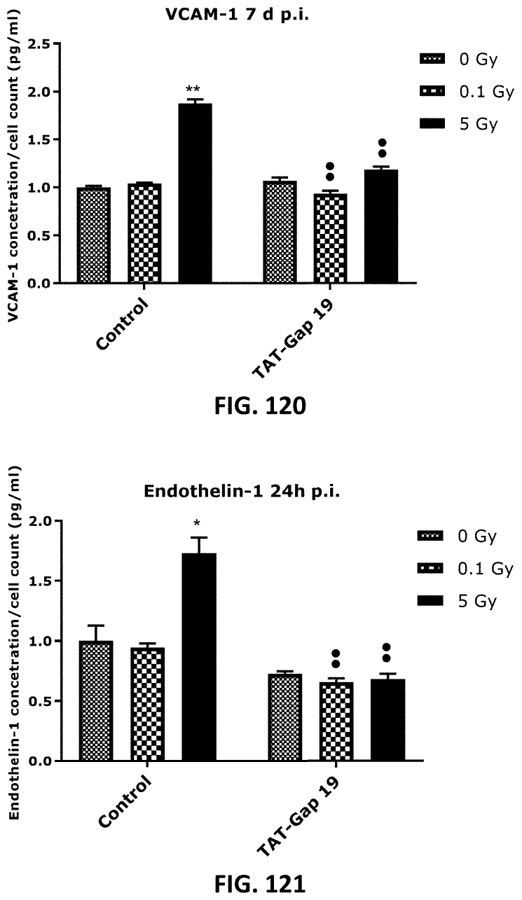

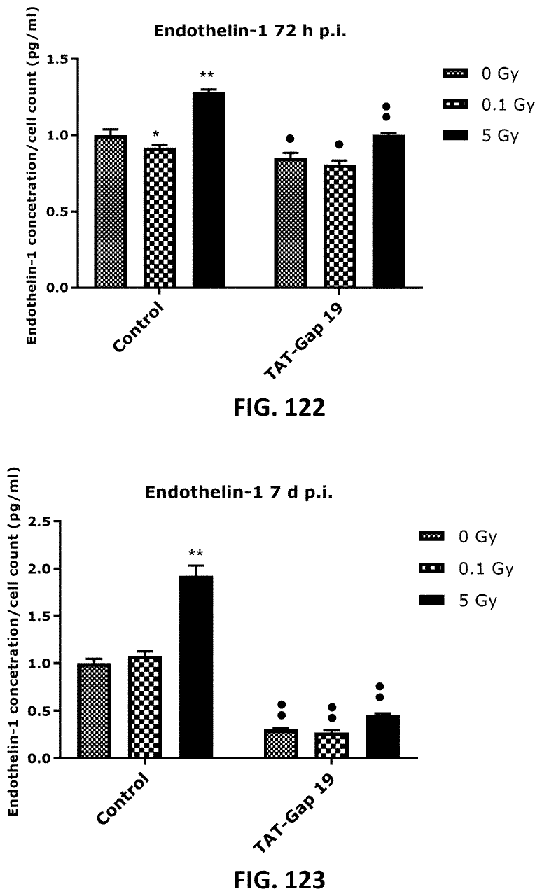

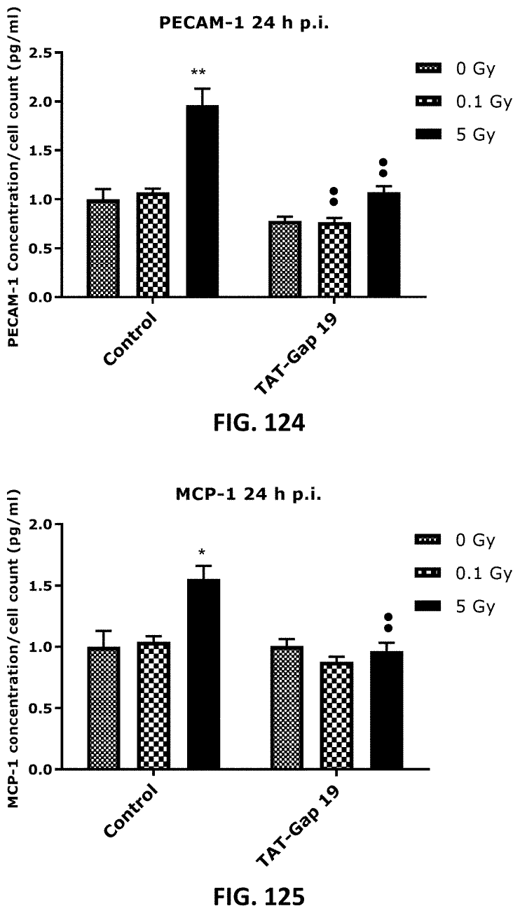

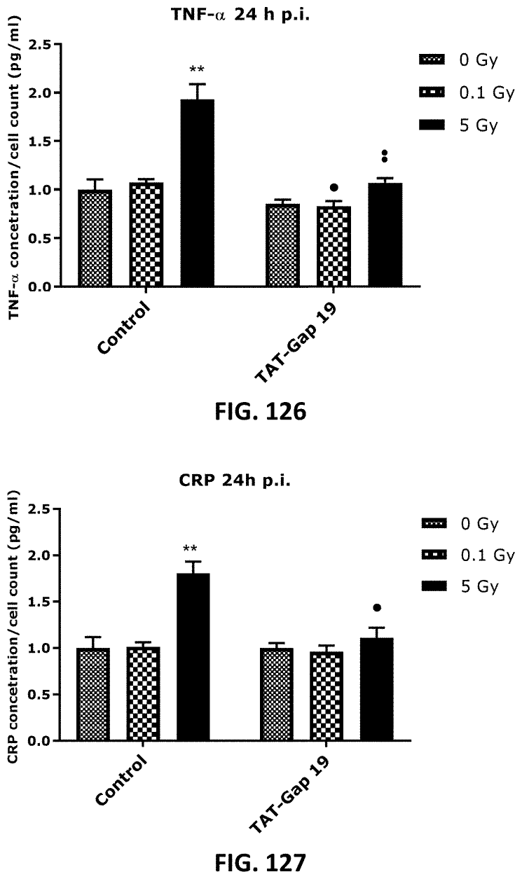

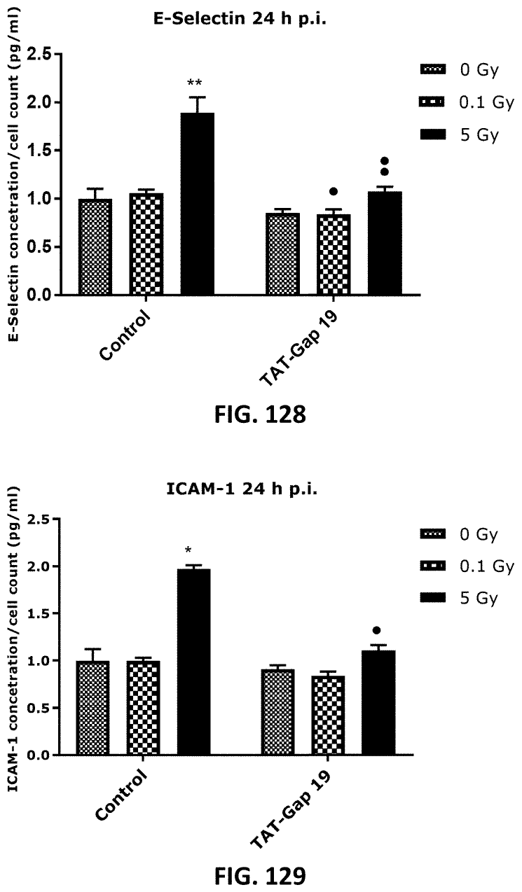

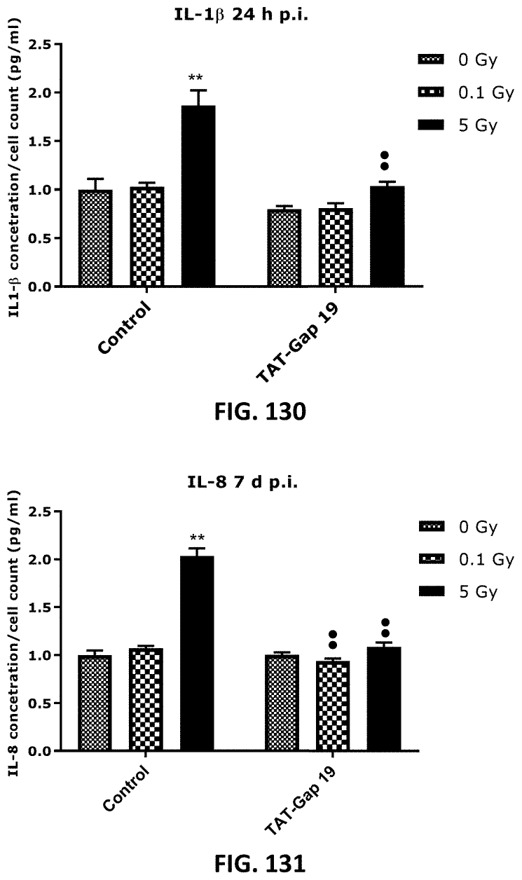

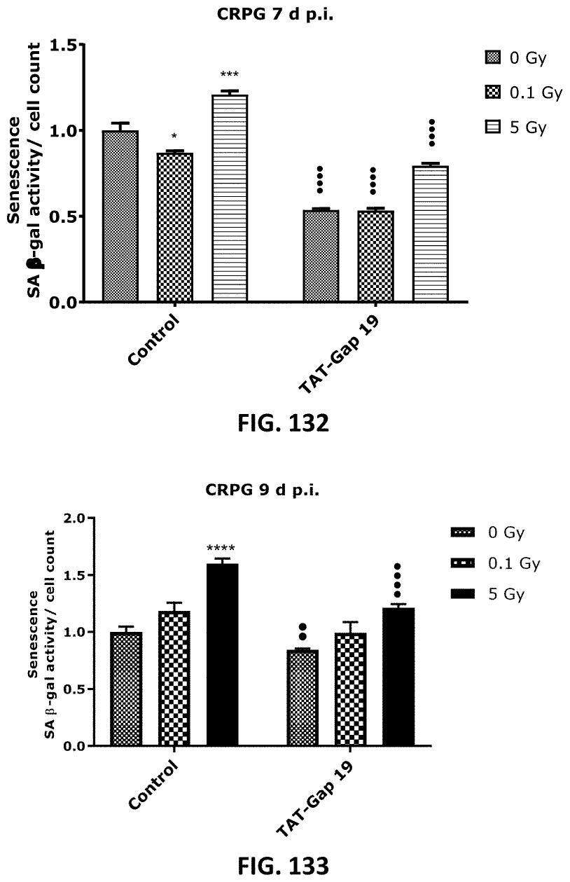

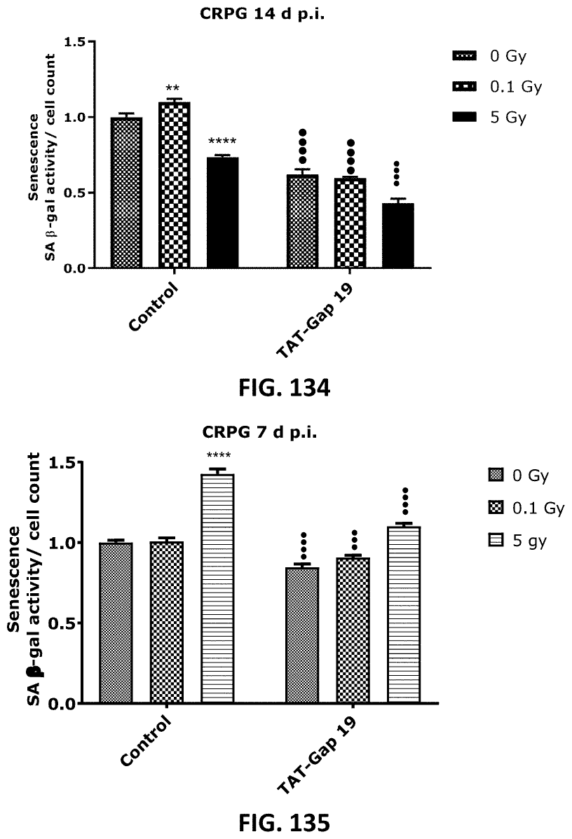

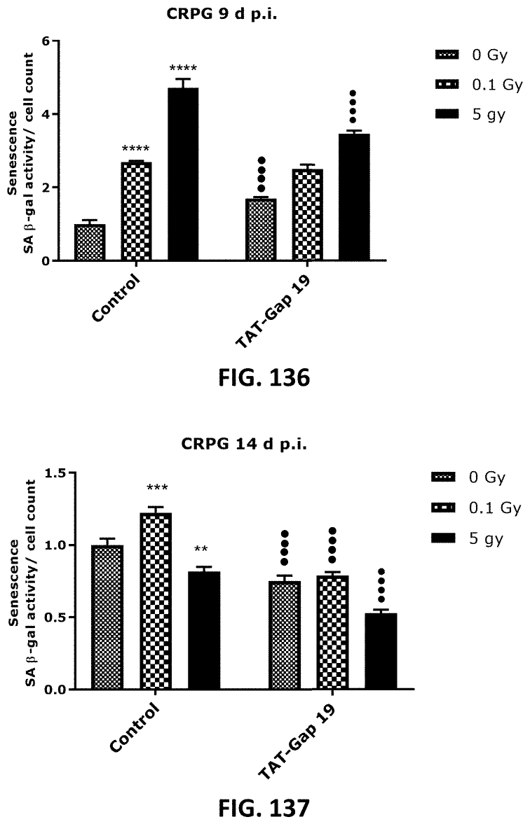

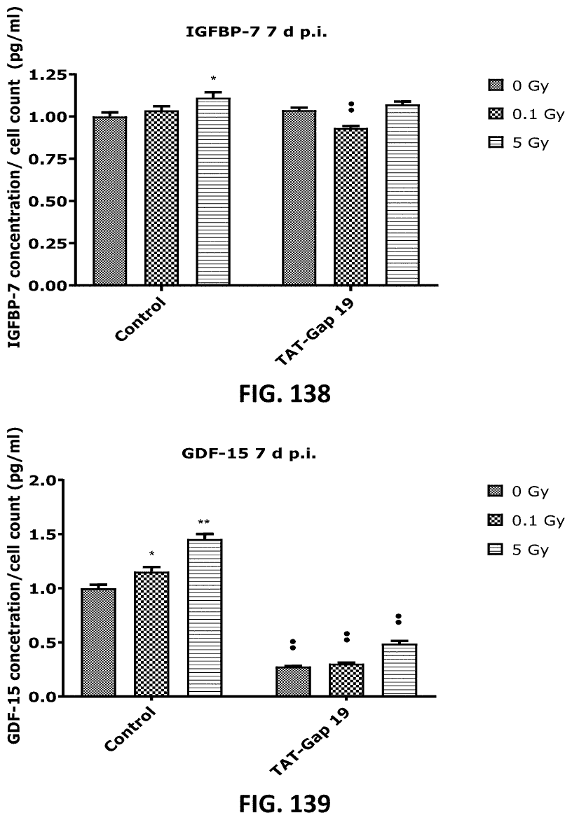

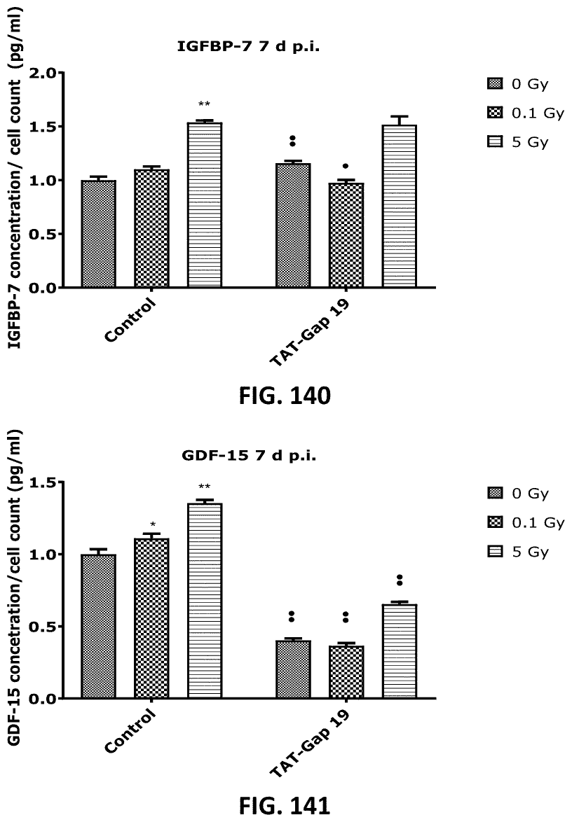

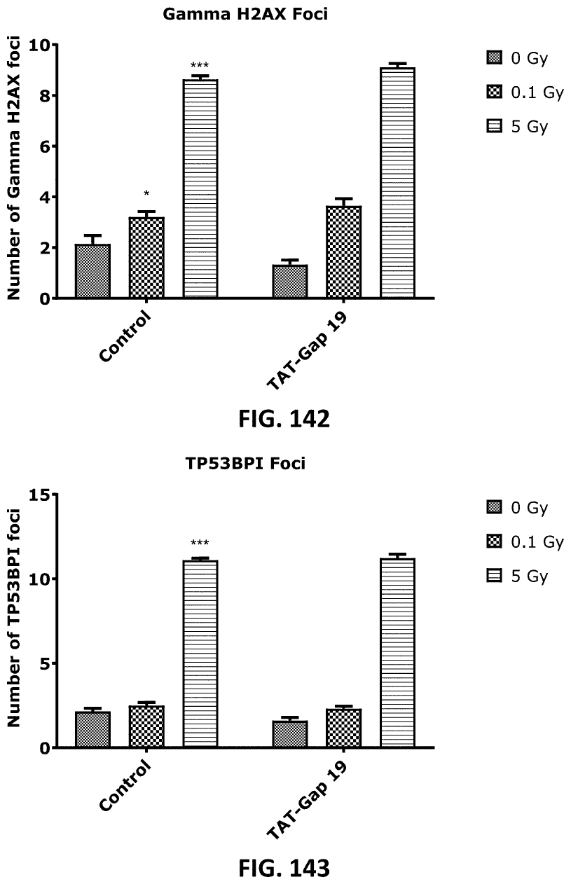

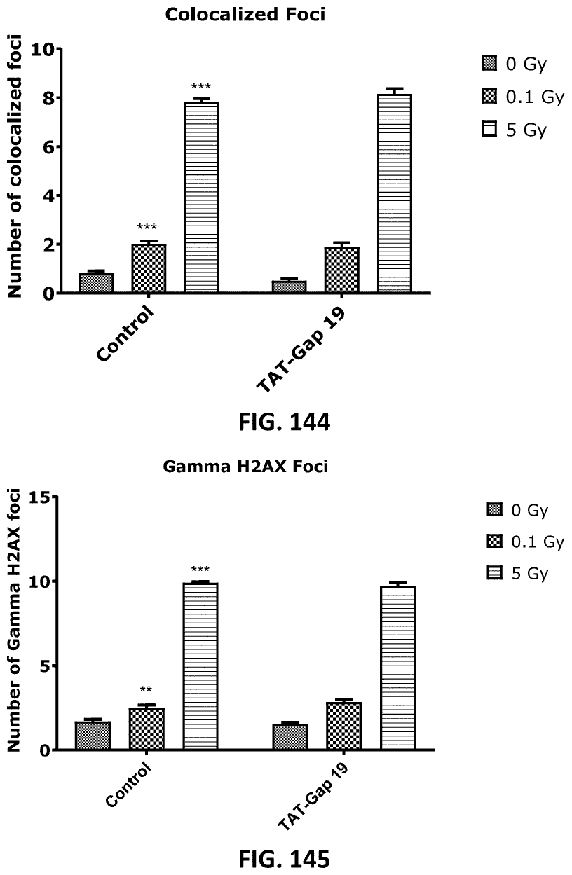

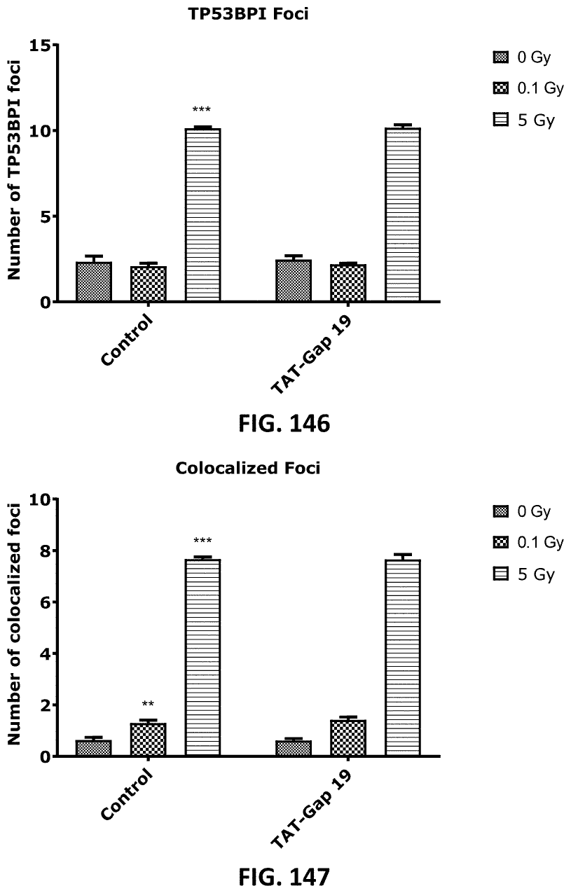

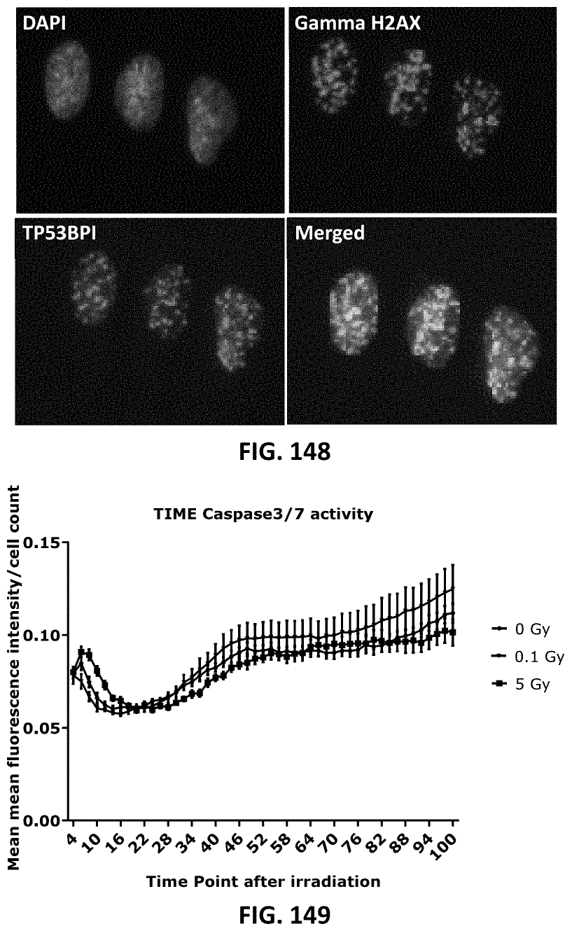

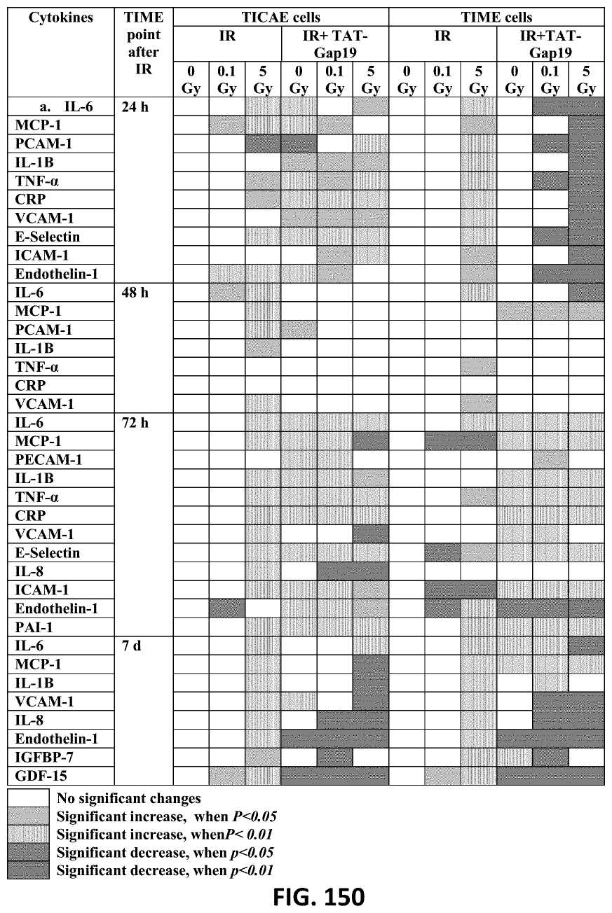

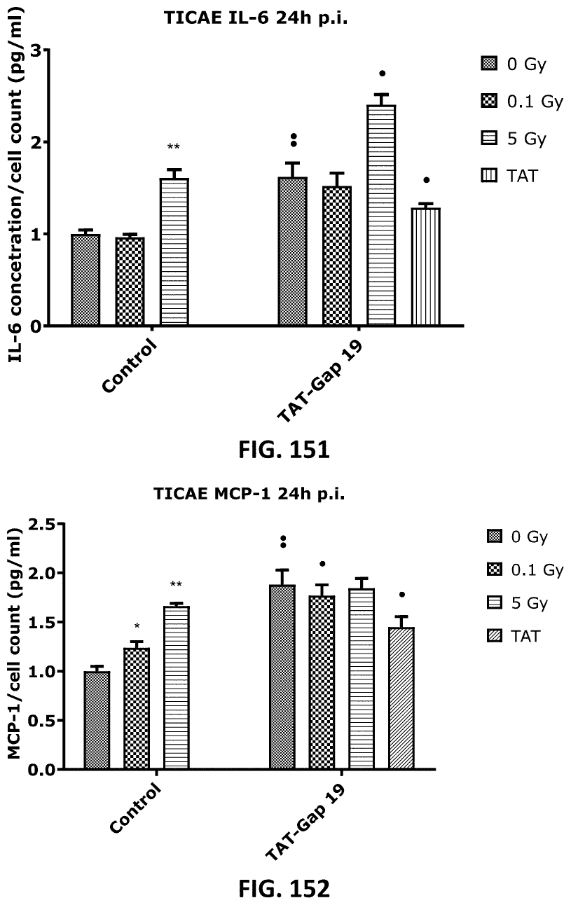

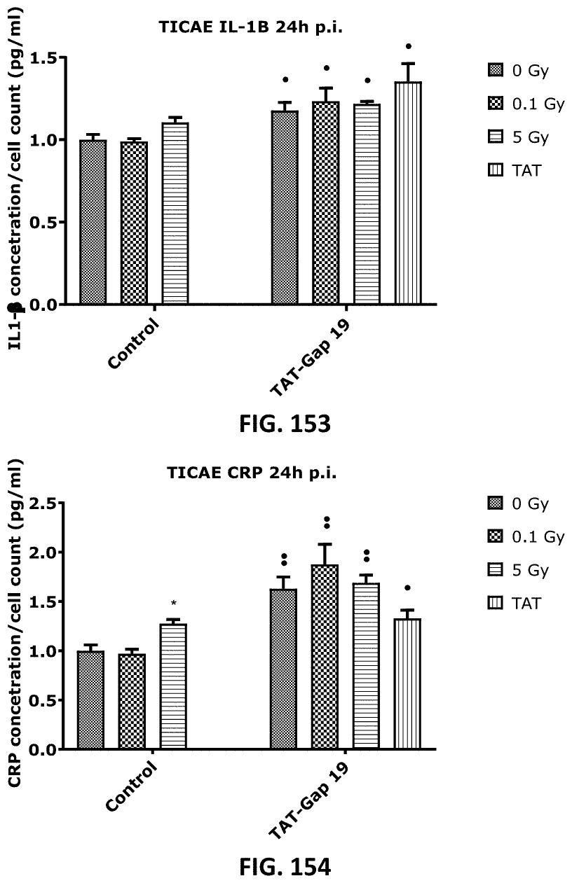

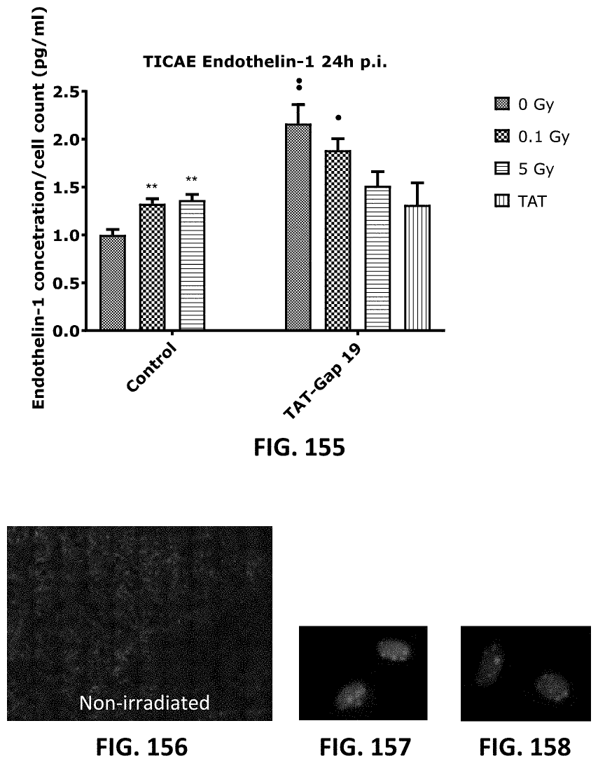

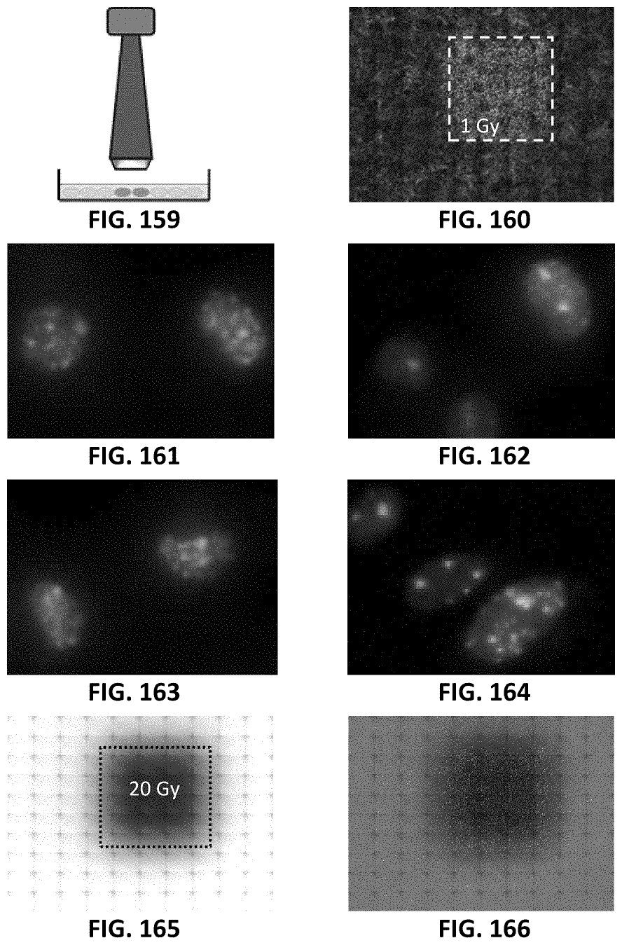

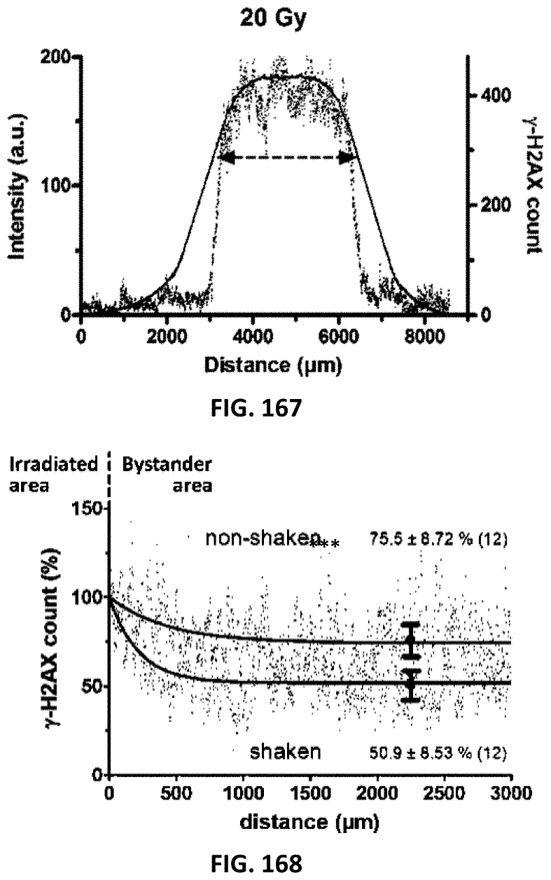

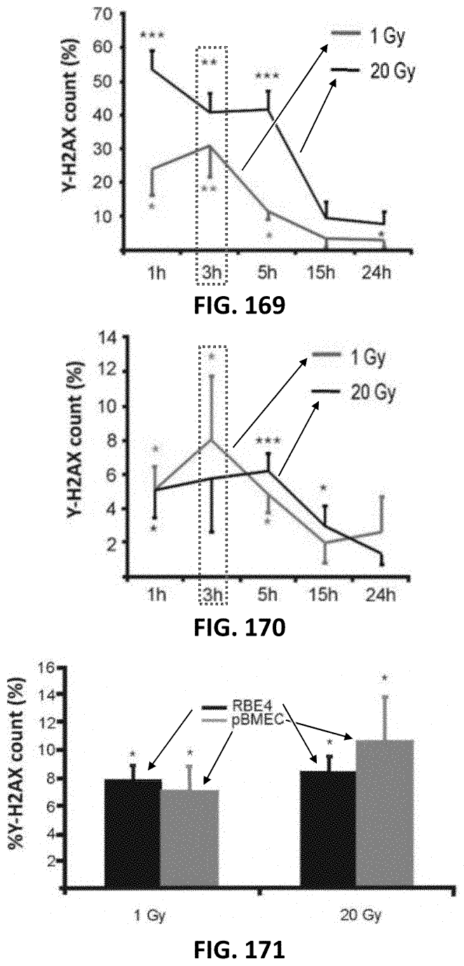

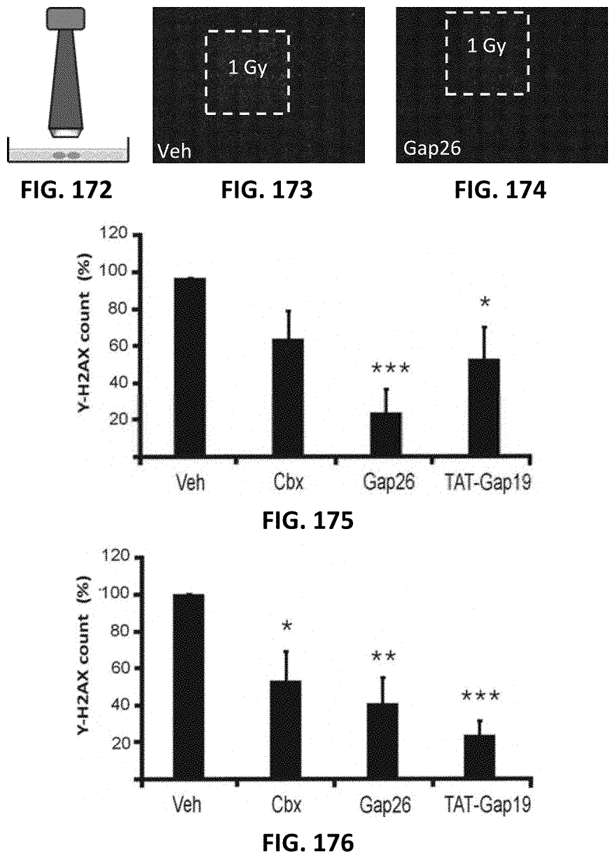

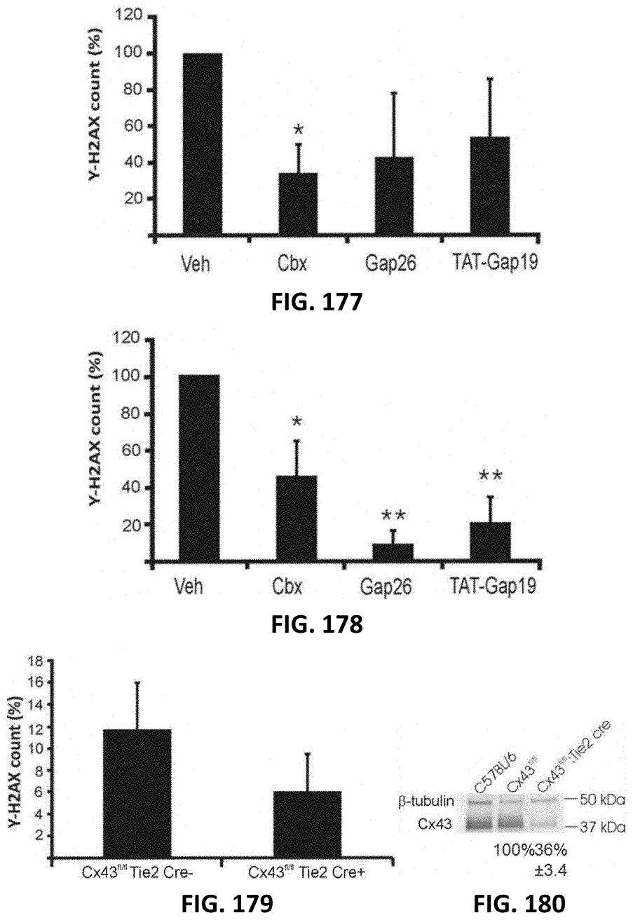

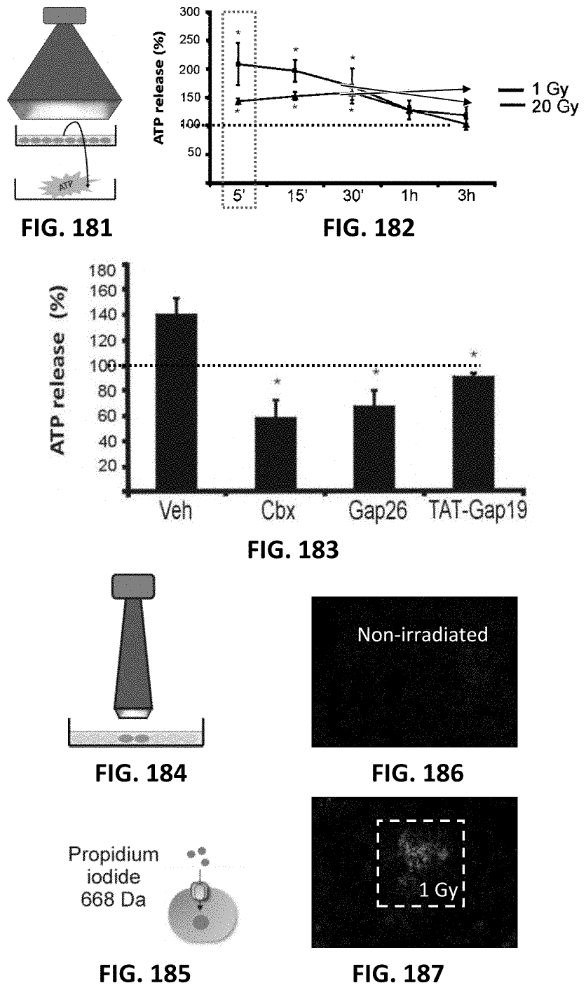

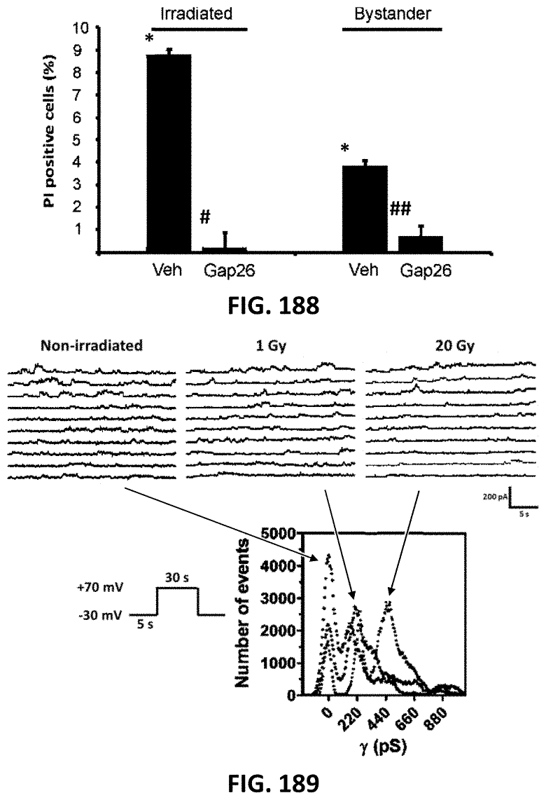

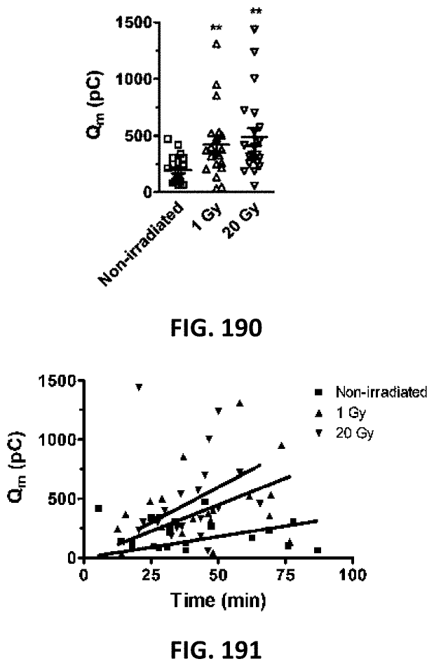

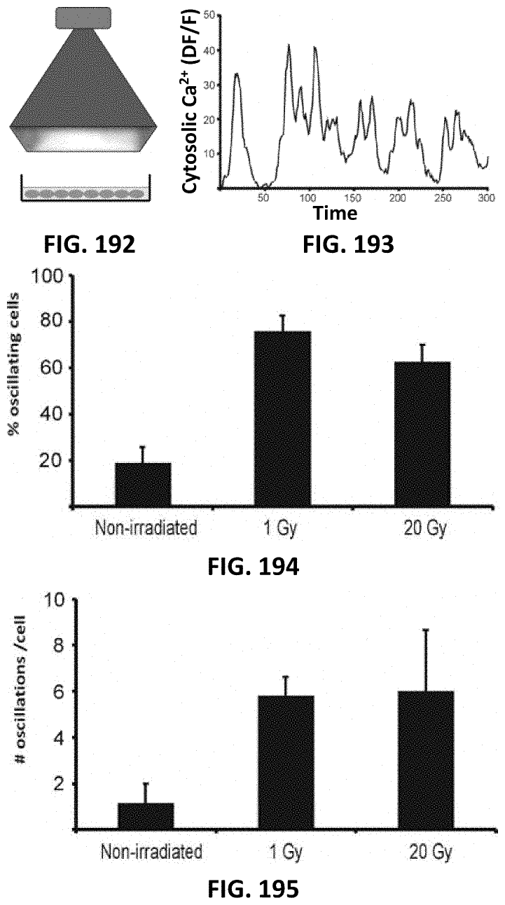

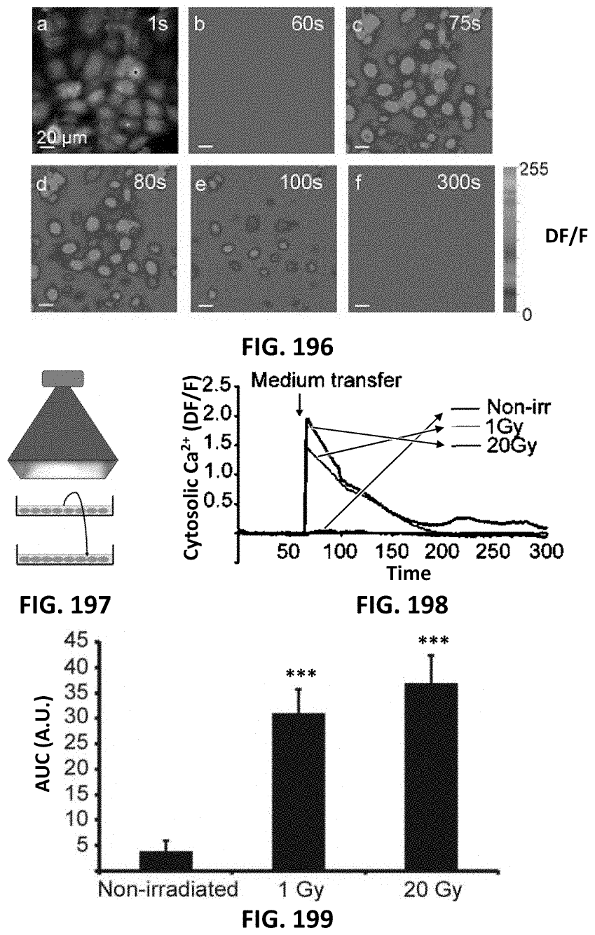

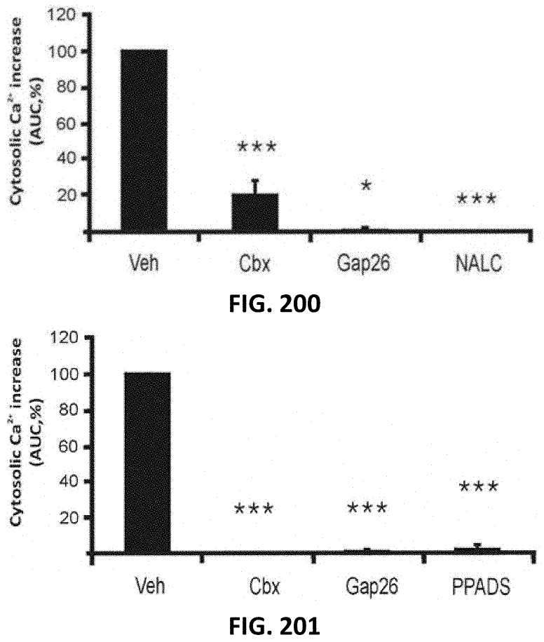

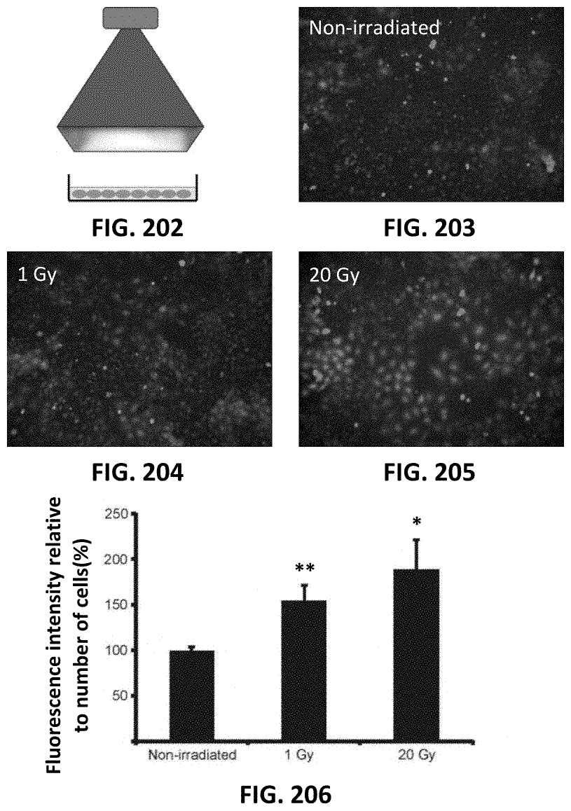

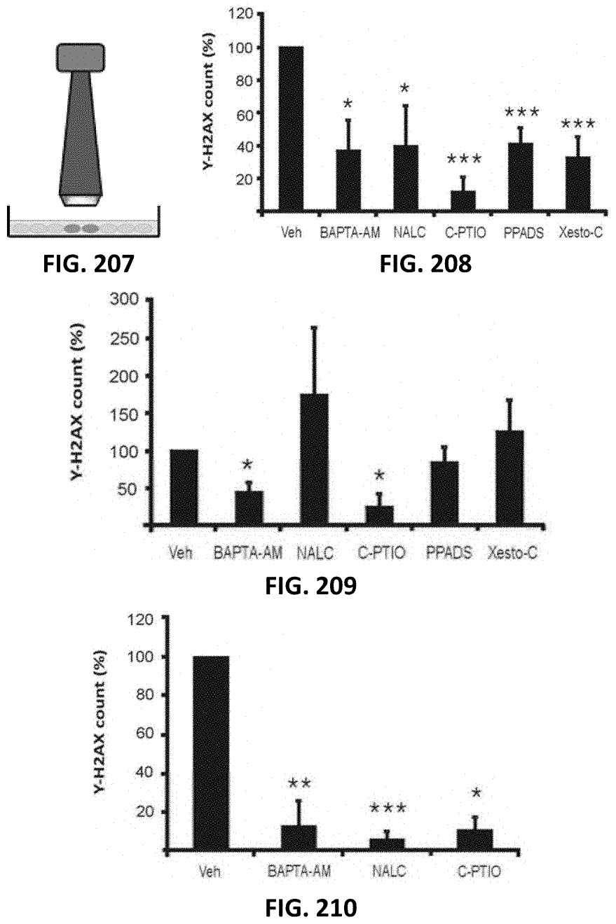

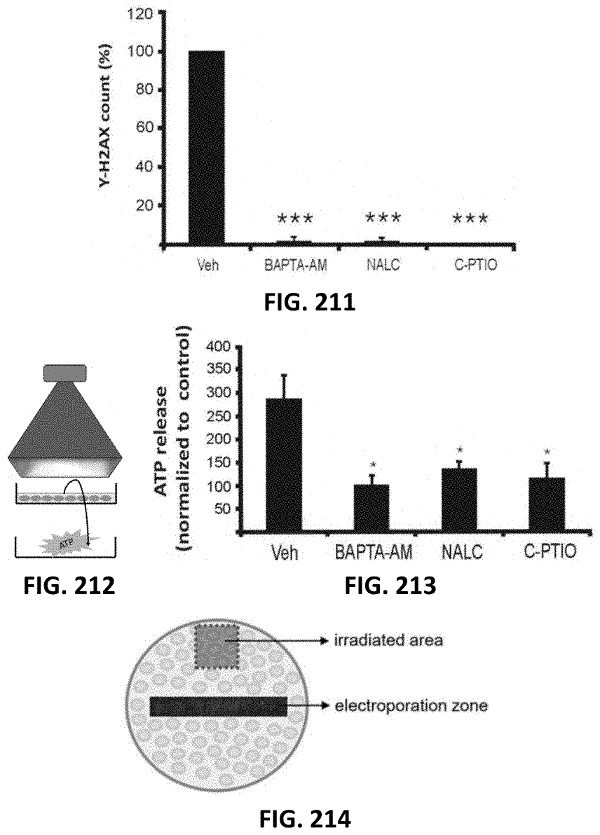

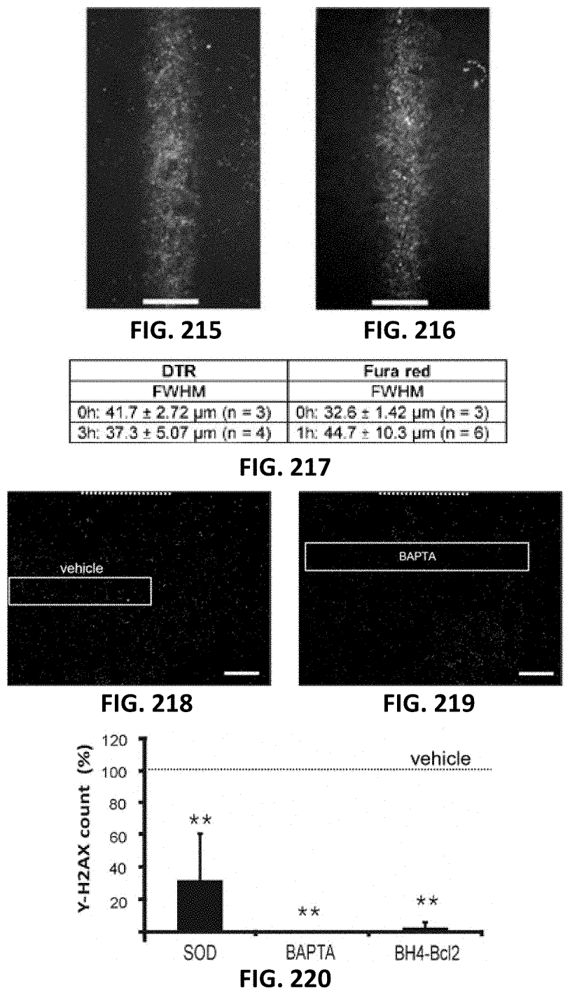

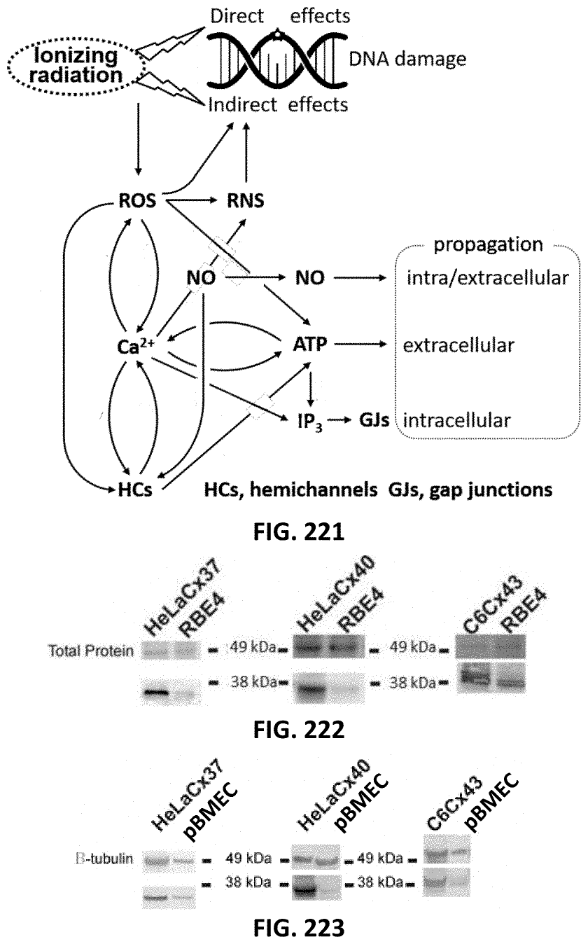

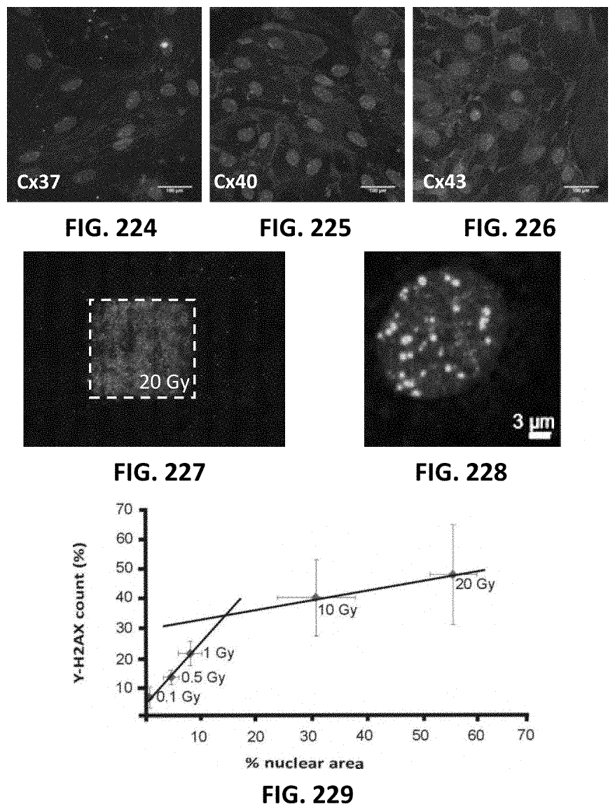

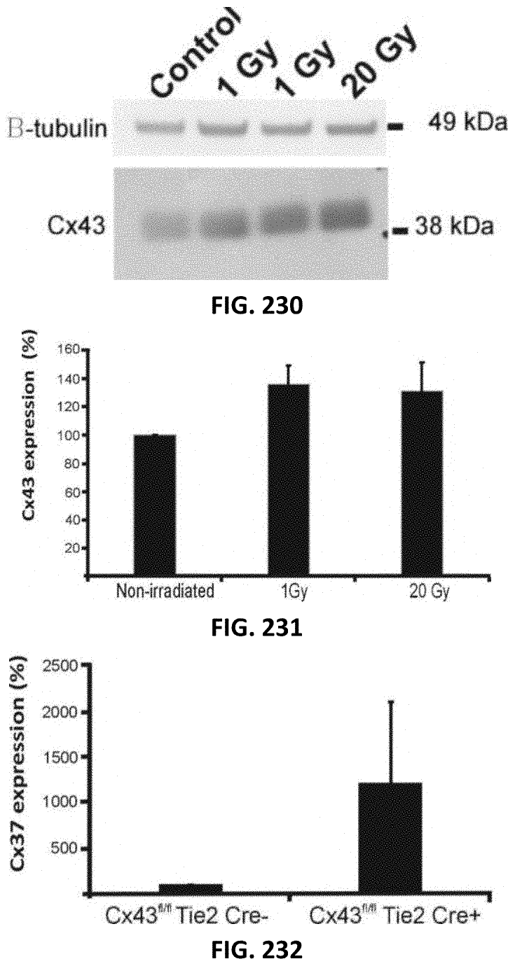

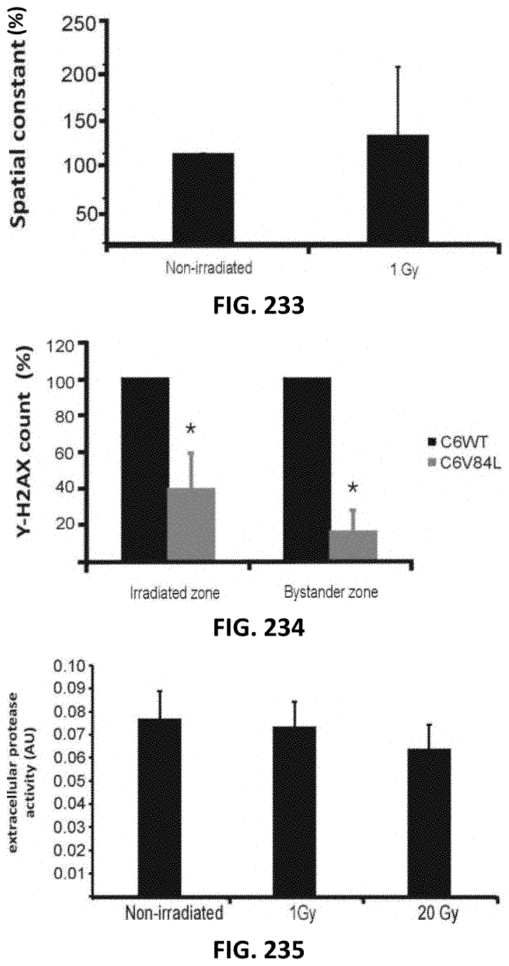

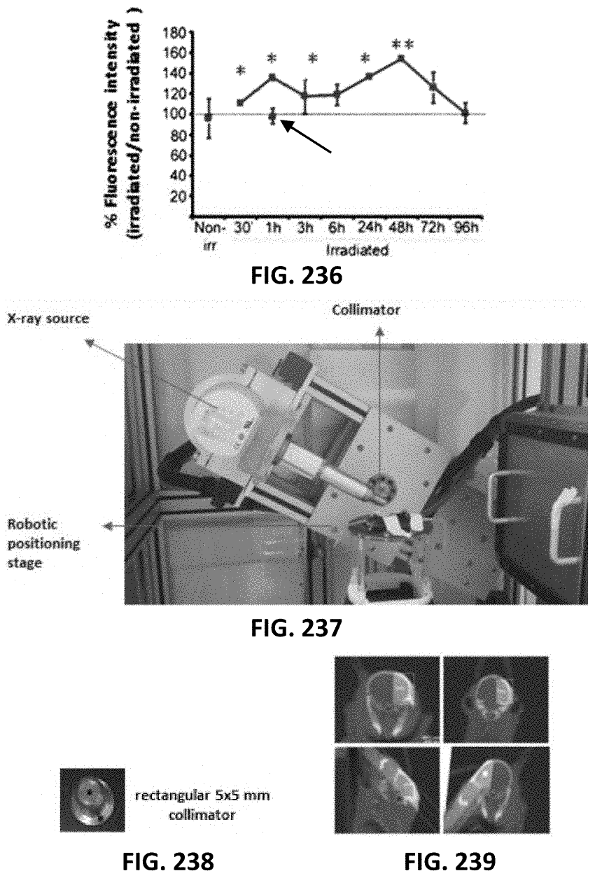

After exposure of TICAE and TIME cells to a single dose of 0.1 or 5 Gy, SLDT assays were performed at 6 h and 72 h p.i. At 6 h p.i., a significant increase in 6-CF diffusion area was observed for the 5 Gy dose in TICAE cells compared to control non-irradiated cells (FIG. 28). At 72 h p.i., a significant increase in 6-CF diffusion area was observed for 0.1 and 5 Gy in both TICAE and TIME cells compared to control non-irradiated cells (FIG. 28 and FIG. 30). Carbenoxolone inhibited the dye spread in all cases. These results reflect a radiation-induced increase in gap junction communication, even for low doses at 72 h p.i. in TICAE and TIME cells (summarized in FIG. 57).

b. Fractionated Irradiation

The 72 h p.i. time point was selected to evaluate the effect of fractionated irradiation on dye spread (fractionated irradiation as applied in the preceding experiments), and to compare it to single exposure effect. For TICAE and TIME cells, only the 5 Gy dose resulted in significantly increased dye spread (FIG. 32 and FIG. 34). For TICAE cells, there were no differences between fractionated and single exposures (FIG. 33). In TIME cells, fractionated irradiation with 0.1 Gy was significantly less potent in increasing dye spread compared to single irradiation (FIG. 35).

### 4. Radiation-Induced Alterations in Hemichannel Function

### 4.1. Assessment of Hemichannel Opening by Measuring Extracellular ATP Release

We first assessed hemichannel opening by determining ATP release in response to 0.1 Gy and 5 Gy irradiation at 1 h, 6 h and 72 h p.i. and tested the effect of hemichannel blockade with TAT-Gap19 which blocks hemichannels composed of Cx43 while it does not inhibit gap junctions.

a. Single Irradiation

In TIME cells, the effects were most strong and both 0.1 and 5 Gy doses triggered significant ATP release at all time points (1, 6 and 72 h) (FIG. 39, FIG. 40 and FIG. 41). In TICAE cells, only 5 Gy induced significant ATP release, and in all cases, except the 1 h p.i. case, TAT-Gap19 significantly reduced the ATP release (Error! Reference source not found., FIG. 37 and FIG. 38). In most cases, TAT-Gap19 significantly inhibited the radiation-induced ATP release but no inhibition was observed for the 1 h p.i. time point for the 0.1 and 5 Gy doses in TICAE and the 0.1 Gy dose in TIME cells. This lack of TAT-Gap19 effect may indicate ATP release via pathways other than Cx43 hemichannels, which may predominate in the early 1 h period after irradiation. FIG. 57 summarizes the responses to single irradiation that were inhibited by TAT-Gap19, thereby reflecting Cx43 hemichannel involvement.

b. Fractionated Irradiation

The 72 h p.i. time point was selected to evaluate the effect of fractionated irradiation on ATP release, and to compare it to single exposure effect (FIG. 42-FIG. 45). As observed with single irradiation, TIME cells showed significant ATP release for both 0.1 and 5 Gy doses while responses in TICAE cells were only significant for 5 Gy. In both cells, TAT-Gap19 significantly inhibited the 5 Gy responses only, presumably because the responses at 0.1 Gy were either non-significant (TICAE) or small (TIME cells) (FIG. 42 and FIG. 44). Interestingly, fractionated irradiation in TICAE cells induced significantly more ATP release at 0.1 Gy compared to single irradiation (FIG. 43 and FIG. 45).

### 4.2 Assessment of Hemichannel Opening Via Dye Uptake Assays

To further assess hemichannel opening, the previous experiments were complemented with dye uptake assays using propidium iodide (PI) and 10 kDa dextran fluorescein as reporter dyes. As an additional control, we also verified the effect of Cx43 hemichannel blockade with TAT-Gap19.

a. Single Irradiation

A radiation-induced increase in dye uptake was mainly significant for the 5 Gy dose at 1 h, 6 h and 72 h p.i. in both TICAE and TIME cells; in TIME cells, the lower 0.1 Gy dose also gave significant dye uptake (FIG. 46 to FIG. 51). Collectively, these responses were very similar to those observed in the ATP release assays. TAT-Gap19 inhibition of these responses was most clear and significant at the 72 h p.i. time point for both cells. Significant inhibition by TAT-Gap19 was also seen at 1 h p.i. in TICAE cells exposed to 5 Gy. In line with the ATP release data, TAT-Gap19 was less efficient in inhibiting dye uptake in the early post irradiation period, here including both the 1 h and 6 h time points. This may indicate PI dye uptake via poorly selective channels other than Cx43 hemichannels and activated by irradiation. FIG. 57 summarizes dye uptake responses that were inhibited by TAT-Gap19, thereby reflecting Cx43 hemichannel involvement.

b. Fractionated Irradiation

The 72 h time point was selected to evaluate the effect of fractionated irradiation on PI dye uptake, and to compare it to single exposure effect. Fractionated irradiation induced an increase in dye uptake in TICAE and TIME cells exposed to 5 Gy but here TAT-Gap19 had no effect (FIG. 52 and FIG. 54). Interestingly, TICAE cells exposed to fractionated 5 Gy irradiation induced significantly stronger dye uptake compared to a single irradiation scheme (FIG. 53 and FIG. 55).

### 5. Principal Component Analysis

In order to have a concise view on the major effects observed, we performed principal component analysis (PCA) for Cx40 and Cx43 gene expression and protein level in both TICAE and TIME cells after single irradiation over the 6 h to 14 d p.i. time period (FIG. 56). This analysis indicated a dose-dependent separation between the radiation doses used (0.1, 0.5 and 5 Gy), which significantly shifted the PCA profiles along the positive side of the first component axis (p<0.001) reflecting dose-dependent alterations in Cx40 and Cx43. In particular, the dose significance started from the 0.5 Gy dose (p<0.00017) followed by the 5 Gy dose (p<0.001) all compared to the non-irradiated control. In addition, a time factor could explain the profile shifts of the irradiated cells along the positive side of the second component axis, with statistical significance (p<0.04). In particular, the late time points group together with the non-irradiated control, i.e. the effect was neutralized and the neutralization effect apparently became more pronounced at 14 d for 0.1 Gy condition. On the other hand, such waxing and waning of the radiation effect was not observed for the 0.5 Gy condition and more pronounced for the 5 Gy condition where the profiles shifted away from the control up to the last measurement at 14 d post exposure (i.e. the effect was persistent) (FIG. 56).

### DISCUSSION

Growing epidemiological data suggest that endothelial cell irradiation induces atherosclerosis. However, the underlying mechanisms are not fully understood. Although connexins were reported to be sensitive to IR and to play a role in atherosclerosis development, their role in radiation-induced atherosclerosis has been poorly explored. Here, we aimed to investigate changes in connexin expression and channel function in response to IR, as a putative mechanism leading to radiation-induced endothelial cell dysfunction, an early event in the atherosclerotic process. We report that single and fractionated X-ray irradiation of coronary artery and microvascular endothelial cells, at doses that can be received after serial diagnostic procedures (0.1 Gy) or as out-of-field exposure after radiotherapy (5 Gy), significantly alters endothelial connexin gene expression, protein levels, gap junctional dye coupling and hemichannel function. Below we discuss these findings in more detail.

We demonstrate changes in Cx37 gene level, consisting of an early (6 h) upregulation at 0.1 Gy followed by a dose-dependent downregulation in TICAE cells and fluctuating responses in TIME cells. These alterations indicate that Cx37 modulation in response to IR is cell line specific, possibly related to distinct endothelial properties at different sites of the vascular tree. For Cx40, gene expression was up at the early time point for the 0.1 Gy dose in the two cell types, as observed for Cx37. Higher doses mainly gave downregulation in TICAE cells and showed more variable responses in TIME cells. Interestingly, the early transient upregulation at 0.1 Gy for both Cx37 and Cx40 genes, observed in both cell types, may reflect possible early protective effects. Protective effects after low dose ionizing irradiation in endothelial cells, such as diminished leukocyte adhesion and enhanced antioxidative defence were reported before. In contrast to the sometimes fluctuating gene expression responses, changes in protein levels of Cx40 were coherent, demonstrating dose-dependent downregulation, significant from low dose (0.1 Gy) at different time points in TICAE and TIME cells. For Cx43, gene expression and protein levels grossly corresponded and demonstrated a dose-dependent elevation at different time points in TICAE and TIME cells. We furthermore found radiation-induced elevation of phosphorylated and hyperphosphorylated Cx43 forms for both cell types. Interestingly, a previous study showed that endothelial Cxs have a distinct temporal expression pattern over time in non-irradiated conditions. In summary, as indicated in PCA (FIG. 56), there is a dose-dependent response in Cx40 and Cx43 after radiation exposure, giving the strongest response at 5 Gy. Additionally, the profile of the irradiated cell responses neutralized after 14 d for 0.1 Gy, while 0.5 Gy and higher gave a more permanent change within the 14 d observation window.

As radiotherapy is delivered to the tumour in multiple radiation fractions, assessing the effect of fractionated irradiation on vascular endothelial cells is of clinical importance. As indicated in PCA (FIG. 71) both single and fractionated irradiation induced the dose-dependent response in Cx40 and Cx43, with persistent changes for 5 Gy condition. However, there are some differences between single and fractionated irradiation response that are more pronounced in TIME cells, as indicated in FIG. 58, suggesting that fractionated irradiation response is cell type dependent. However, it worth mentioning that the comparison between single and fractionated irradiation is limited to the radiation response after normalizing the controls (fold changes). The overall observed decreased expression of atheroprotective Cx37 (gene) and Cx40 (gene/protein), and the increase in the proatherogenic Cx43 (gene/protein) induced by radiation exposure may positively modulate susceptibility to atherosclerosis as delineated in the introduction.

In addition to alterations at the gene and protein level, we also observed functional changes at the level of both gap junctions and hemichannels. Overall, it was clear that irradiation increased gap junctional communication as assessed by dye coupling, which was most obvious at 72 h post single and fractionated irradiation in both TICAE and TIME cells. Taken together, the prominent presence of Cx43 in vascular endothelial cells of the major arteries, combined with the variable observations for Cx37 gene alterations and the decreased level of Cx40 protein, the observed increase in gap junctional coupling is likely caused by the increased Cx43 expression, possibly in combination with the increase in phosphorylated forms that act to enhance gap junctional communication. At the level of hemichannels, we used ATP release and propidium iodide dye uptake assays to estimate functional alterations. To further increase the robustness of the responses, we used TAT-Gap19, a Cx43 hemichannel inhibitor not inhibiting gap junction channels, to determine whether the responses were Cx43 hemichannel-related. FIG. 57 clearly demonstrates an overall increased hemichannel function (ATP release and dye uptake) in the two cell lines for 0 Gy at the 72 h time point. Irradiation with 5 Gy also showed early 1 h responses in the two cell types. In addition, fractionated irradiation increased hemichannel function (ATP release and dye uptake) mainly at 5 Gy. Thus, hemichannel function was, as gap junctional function, increased in response to single and fractionated irradiation. As concluded for gap junctions, the increased hemichannel function may relate to increased post irradiation Cx43 expression and its phosphorylated forms. However, unlike gap junctions that are normally open, hemichannels are normally closed and need a trigger to open the channels. Connexin hemichannels open in response to physiological and pathological triggers, including changes in membrane potential, changes in intracellular or extracellular Ca²⁺ concentration, redox alterations, post-translational alterations including phosphorylation and nitrosylation, ischemia and inflammation. Cx43 hemichannel opening, as reported by ATP release assays, has also been demonstrated in B16 melanoma cells in response to γ-rays. X-rays interact with water to form ROS, which acts as a trigger for Cx43 hemichannel opening. In addition, X-rays are known to interact with cysteine, oxidizing it to cystine. Cx43 has 3 cysteines in each extracellular loop and several Cys residues inside the cell, including the C-terminal tail that is crucial for regulating hemichannel function. S-nitrosylation, possibly by NO acting at CT-located Cys residues is again a known trigger of hemichannel opening. Uncontrolled hemichannel opening results in excessive entry of Na⁺ and Ca²⁺, ATP leakage and loss of cell-essential metabolites, which in turn can trigger intracellular events, such as inflammation, NO production and activation of apoptotic caspases. Several studies demonstrated that excessive hemichannel opening contributes to different pathological processes including spread of bystander signalling such as inflammation and oxidative stress, blood-brain barrier opening and cardiac ischemia/reperfusion injury. Furthermore, ATP released via hemichannels and other pathways is a danger signal to the immune system that triggers intracellular Ca²⁺ signalling and is known to activate the NLRP3 inflammasome. DNA damage, oxidative stress, apoptosis and inflammation are known mechanism to be involved in the pathogenesis of radiation-induced atherosclerosis. These radiation damaging responses may spread through gap junction intercellular communication and paracrine signalling mediated via hemichannel into neighbouring cells, possibly amplifying endothelial cell damage.

### Conclusion

In conclusion, to the best of our knowledge, we show for the first time that exposure of coronary artery and microvascular endothelial cells to single or fractionated X-rays, induced an acute and persistent dose-dependent decrease of atheroprotective Cx40 and increase of the proatherogenic Cx43 gene and protein levels. In addition, such radiation exposures increased gap junctional communication and induced acute and long-lived hemichannel opening, the latter considered as a pathological condition. We believe that radiation-induced increased endothelial gap junctional coupling and hemichannel function leads to endothelial dysfunction, which is an early marker for atherosclerosis.

### Example 2: Connexin43 Hemichannels Contribute to Radiation-Induced Endothelial Cell Damage

### Materials and Methods

### J. Cell Culture

Two human endothelial cell lines; Telomerase Immortalized human Coronary Artery Endothelial cells (TICAE) from the European Collection of Authenticated Cell Cultures (ECACC), and Telomerase Immortalized human Microvascular Endothelial cells (TIME) from the American Type Cell Culture (ATTC), were grown in MesoEndo Cell Growth Medium (Sigma-Aldrich Co. LCC, Diegem, Belgium) in a humidified incubator at 37° C. supplemented with 5% CO2. Cells were split every three days with 0.05% trypsin supplemented with 0.02% ethylenediaminetetraacetic acid (EDTA). The passage number 28 until passage 36 was used in all the experiments. Moxi Z Mini Automated Cell Counter (ORFLO Technologies, Ketchum, Id., USA) was used to count the cells. Cells were not passaged after irradiation for all experiments.

### 2. Irradiation

TICAE and TIME cells were irradiated at 100% confluence with 0.1 Gy and 5 Gy single X-rays doses at a dose rate of 0.5 Gy/minute with a vertical, point source X-ray beam using a Xstrahl RX generator (320 kV, 12 mA, 3.8 mm Al and 1 mm Cu) (Camberley, UK).

### 3. Intracellular ROS Detection

### 3.1 Flow Cytometry Detection

TICAE and TIME cells were seeded in a 6-well plate at a density of 250000 cells/well. Three days later, cells reached 100% confluence. At 30 minutes before X-ray exposure (0.1 and 5 Gy), cells were washed with PBS to remove traces of the original medium. Afterwards, HMS supplemented with 25 mM HEPES (Sigma-Aldrich Co. LCC, Diegem, Belgium) and 10 μmol/L of the CM-H2DCFDA dye (Sigma-Aldrich Co. LCC, Diegem, Belgium) was added to the cells, and 100 μM TAT-Gap19 (Genosphere Biotechnologies, Paris, France) was added to block the Cx43 hemichannels. After irradiation, cells were washed with PBS and a 0.05% trypsin solution supplemented with 0.02% EDTA was used to detach the cells, followed by a 5 minute incubation time (37° C., 5% CO2). After trypsinization, the cells were transferred into a 1.5 mL tube and centrifuged for 0 minutes at 200 RCF. The supernatant was discard and the pellet was re-suspended in MSS without Ca2+ and Mg2+(Sigma-Aldrich Co. LCC, Diegem, Belgium). Finally, 1 μg/mL PI was added to each sample to exclude the dead cells. A positive control, 20 μmol/L tert-butyl hydroxyperoxide (tBHP), was added to the cells and incubated for 15 min. Intracellular ROS was assessed by analyzing the fluorescent signals using BD Accuri™ c6 Flow Cytometry (BD Biosciences, San Jose, Calif. USA). At least 10 000 events were measured. Before analysis, a color compensation for the CM-H2DCFDA and PI staining was performed, by using single color controls (cells treated with CM-H2DCFDA or PI, and untreated cells) and adjusting the fluorescent signals in the FL1 and FL3 channels. Gating was adjusted based on CM-H2DCFDA positive and PI negative condition in control non-irradiated cells.

### 3.2 IncuCyte Live Cell Imaging

In order to further assess intracellular ROS production, IncuCyte live cell imaging was used to have fast imaging in large number of replicates over multiple time points. TICAE and TIME cells were seeded in 96-well plate in 16 replicates at a density of 10000 cells/well. Three days later, cells reached 100% confluence. At 30 minutes before irradiation, a fresh MesoEndo Cell Growth Medium without phenol red (Sigma-Aldrich Co. LCC, Diegem, Belgium) supplemented with 10 μmol/L of the CM-H2DCFDA dye (Sigma-Aldrich Co. LCC, Diegem, Belgium) was added to the cells, with or without 100 μM TAT-Gap19 (Genosphere Biotechnologies, Paris, France). Fluorescence signals were measured at 45 min, 2 h and 3 h after irradiation (CM-H2DCFDA dye became saturated due to photoactivaton, and leaked outside the cells after this time) using the IncuCyte ZOOM® system (Essen BioScience, Ann Arbor, Mich., USA), 10× objective and FITC filter. Phase contrast imaging was performed in order to count the cells. A positive control of 10 μmol/L tert-butyl hydroxyperoxide (tBHP), was added to the cells directly before imaging. Images were analysed making use of the software package provided by the manufacturer; we used a training set of images from different doses to define the image processing procedure. The so called Top-Hat background subtraction method was applied by measuring the radius of the fluorescence object (30 μm), and adjust a threshold of 1.5 (above the local background fluorescent intensity level), as described in IncuCyte™ Background Fluorescence Technical Note. These process definitions were applied for all conditions in TICAE and TIME cells.

### 4. Cell Death Assessment

### 4.1 Annexin V and Caspase 3/7

TICAE and TIME cells were seeded in 96 well plates at a density of 10000 cells/well in 8 biological replicates. After three days, cells were at 100% confluence. At 30 minutes before X-ray exposure, cells were refreshed with 200 μl medium supplemented with Caspase 3/7 reagent (1:1000 dilution) and Annexin V reagent (1:200 dilution) (Essen Bioscience, United Kingdom) with or without 100 μM TAT-Gap19 (Genosphere Biotechnologies, Paris, France). Fluorescence signals were measured from 4 h until 100 h after irradiation by imaging every 2 hours using the IncuCyte ZOOM® system (Essen BioScience, Ann Arbor, Mich., USA). Different channels (TRITC, FITC and phase contrast) and a 10× objective were used. Spectral unmixing set as 8% of red removed from green was applied. The IncuCyte software's processing definition was set to recognize red (Annexin V) and green (Caspase 3/7) stained cells, making use of the Top-Hat background subtraction method. Fluorescence signal was normalized to cell count.

### 4.2 Dextran Fluorescein Dye Uptake Assay

Dextran fluorescein, staining late apoptotic and necrotic cells, was used to validate apoptosis in TIME cells after irradiation. Cells were seeded in a 24-well plate at a density of 1.5×10⁵ cells/well in 6 biological replicates. Three days later, cells reached 100% confluence. Before X-ray exposure with 30 minutes, cells were refreshed with medium with or without 100 μM TAT-Gap19 (Genosphere Biotechnologies, Paris, France). At 6 h and 72 h after irradiation, cells were washed twice with HMS supplemented with 25 mM HEPES (Sigma-Aldrich Co. LCC, Diegem, Belgium) (HBSS-HEPES). Afterwards, the cells were incubated with 200 μM dextran fluorescein (10 kDa) (Life Technologies, Merelbeke, Belgium), dissolved in HBSS-HEPES. After six minutes, the staining was removed by washing six times with HBSS-HEPES. Finally, the fluorescence was measured using the IncuCyte ZOOM® system (Essen BioScience, Ann Arbor, Mich., USA) by using FITC channel and phase contrast to count the cells using a ×10 objective. The fluorescence signal was normalized to cell count.

### 5. Cytokine Detection

TICAE and TIME cells were seeded in a 6-well plate at a density of 2.5×10⁵ cells/well in 5 to 6 biological replicates. Two to three days later, cells reached 100% confluence. At 30 minutes before X-ray exposure, cells were refreshed with medium with or without 100 μM TAT-Gap19 (Genosphere Biotechnologies, Paris, France). At 24 h, 48 h, 72 h and 7 d after irradiation, the supernatant was collected. Medium was changed only for the 7 d time point experiment at the fourth day with fresh medium alone or with medium supplemented with 100 μM TAT-Gap19. For the simultaneous detection of multiple cytokines in the supernatants, the Magnetic Luminex® Assay (R&D systems, Minneapolis, Canada) was used following manufacturer's instructions. Briefly, supernatants, standards and microparticles were incubated into a 96-well plate which was pre-coated with cytokine-specific antibodies. The immobilized antibodies were able to bind the cytokines of interest after 2 h incubation, then the plate was washed and incubation with a biotinylated antibody cocktail specific to the cytokines of interest was performed for 1 h. A second wash was performed to remove the unbound biotinylated antibodies. Further, a streptavidin-phycoerythrin conjugate was added to each well to bind to the biotinylated antibodies. After a final wash, the microparticles were resuspended in buffer and read using the Luminex® MAGPIX Analyzer (R&D systems, Minneapolis, Canada).

In order to normalize to cell number, DAPI staining was performed for the 48 h after irradiation time point. Briefly, cells were washed with phosphate buffered saline (PBS) and fixed with 4% paraformaldehyde (PFA) for 15 minutes at room temperature. Afterwards, the cells were washed with PBS and stained with 1 μg/mL 4′,6-Diamidine-2′-phenylindole (DAPI) dissolved in 1×TBST, 0.005 g/v % TSA blocking powder (PerkinElmer, FP1012) (TNB). After one hour, cells were washed with PBS and DAPI signals were visualized with the Nikon Eclipse Ti automated inverted wide-field epifluorescence microscope equipped with a 5× magnification (Plan Fluor, numerical aperture 0.6) dry objective and a Nikon IE2000-E camera controlled by the MS Elements software. The intensity of the DAPI signals was determined with the FIJI software. Cell count for 24 h after irradiation experiment was performed by Moxi Z Mini Automated Cell Counter (ORFLO Technologies, Ketchum, Id., USA) after trypsinization. For the 72 h and 7 d post-irradiation cell counts, we used IncuCyte ZOOM™ phase contrast imaging based on 49 images taken per well with the 10× objective.

### 6. Senescence Detection by CPRG Assay

TICAE and TIME cells were seeded in 96 well plates at a density of 10000 cells/well in 16 replicates. Three days after, cells were at 100% confluent. At 30 minutes before X-ray exposure, the cells were refreshed with 150 μl medium alone or with medium supplemented with 100 μM TAT-Gap19 (Genosphere Biotechnologies, Paris, France). For 7 d and 9 d post irradiation experiments, the medium was refreshed once, while for 14 d post irradiation experiment the medium was refreshed twice with 200 μl medium alone or with medium supplemented with 100 μM TAT-Gap19. At 7, 9 and 14 days post irradiation, cell number was determined using an Incucyte® ZOOM life cell analysis system and related software (Essen Bioscience, Hertfordshire, United Kingdom). Afterwards, the senescence associated β-galactosidase activity in the cells was determined using the chlorophenol red β-D-galactopyranoside (CPRG) assay as described previously. The cells were washed with PBS and lysed using M-PER™ buffer (Thermo Fisher Scientific, Asse, Belgium). Subsequently, 1×CPRG substrate (2 mM Chloropenol Red β-D-galactopyranoside in CPRG assay buffer containing 50 mM KPO4, 1 mM MgCl2, pH 6) (Sigma-Aldrich, Overijse, Belgium) was added to each well and the plates incubated for 18 h at 37° C. without CO2. A CLARIOstar® microplate reader (BMG Labtech, Temse, Belgium) was used to measure the absorbance at 570 nm.

### 7. DNA Damage Detection

To detect DNA double strand breaks after IR, TICAE and TIME cells were seeded in Nunc™ Lab-Tek™ Chamber Slide (Thermo Fisher Scientific, Asse, Belgium) in 8 replicates, at seeding density of 50 000 cells/well. Two days later, cells reached 100% confluence. At 30 min before IR, a fresh medium was added to the cells with or without 100 μM TAT-Gap19. At 1 h post irradiation, cells were fixed with 2% PFA (Sigma-Aldrich Co. LCC, Diegem, Belgium) and permeabilized with 0.25% Triton X-100 (Sigma-Aldrich Co. LCC, Diegem, Belgium) in PBS. Cells were blocked for 1 h in 1% normal goat serum (Thermo Fisher Scientific, Asse, Belgium) in Tris-NaCl (Perkin Elmer, Brussels, Belgium). Thereafter, staining with 1/300 primary anti-gamma H2AX antibody (Merck-Millipore #05-636) and 1/1000 anti-TP53BP1 antibody (Novus Biologicals #NB100-304) was performed for 1 h at 37° C. After washing 3 times with PBS, secondary Alexa Fluor 488 and 568 antibodies (Life Technologies, Merelbeke, Belgium) were applied for 1 h at 37° C. Finally, the slides were mounted using ProLong Diamond Antifade Mountant with DAPI (Life Technologies, Merelbeke, Belgium), and cells were visualized with an Eclipse Ti automated inverted wide-field epifluorescence microscope (Nikon, Brussels, Belgium) equipped with a 20×Plan Fluor objective (NA 0.6) and an Andor Ixon EMCCD camera, controlled by the MS Elements software. A z-stack of 9 planes axially separated by 1 μm was applied, and 16 fields were captured for each replicate. Analysis were performed with FIJI software using the Cellblocks toolbox.

### 8. Statistical Analysis

Nonparametric two-tailed Mann-Whitney T-test was performed for the analysis of all the experiments, except for dead cell live cell imaging experiment where two-way ANOVA analysis followed by Turky test was used. Statistical significance was considered when P<0.05. Data are presented as mean±standard error of the mean. GraphPad Prism 5.01 (GraphPad Software Inc., La Jolla, Calif. 92037 USA) was used in all the analysis and occasional exclusion of outlier data points were performed using Grubbs' test.

### Results

### 1. Radiation-Induced Oxidative Stress is Supressed by TAT-Gap19

Intracellular ROS production was measured in TICAE and TIME cells in response to exposure to 0.1 Gy and 5 Gy X-rays, making use of flow cytometric measurement of CM-H2DCFDA dye fluorescence. A dose-dependent ROS increase was observed at the 45 min time point in TICAE and TIME cells (FIG. 96 and FIG. 97). Hemichannel inhibition with TAT-Gap19 significantly reduced intracellular ROS generation at 0.1 Gy in TICAE cells (FIG. 96).

In order to obtain more detailed temporal information on ROS production, we used IncuCyte live cell imaging of CM-H2DCFDA fluoresecence in TICAE and TIME cells at 45 min, 2 h and 3 h after IR exposure. In TICAE and TIME cells, a radiation-induced dose-dependent increase in intracellular ROS production was observed from 45 min until 3 h post irradiation (p.i.) (significant for 0.1 and 5 Gy), with the highest response at 45 min p.i. (FIG. 98-FIG. 101). TIME cells showed to be more sensitive, as the extent of ROS production after 45 min of IR was 5 fold larger for the 0.1 Gy dose, and 2 times higher for the 5 Gy dose than in TICAE cells (FIG. 98-FIG. 101). TAT-Gap19 significantly reduced ROS production at both doses (0.1 and 5 Gy) in TICAE cells at 45 min p.i., while in TIME cells, it reduced ROS production only significantly for the 0.1 Gy dose at 45 min p.i. (FIG. 98 and FIG. 99). tert-Butyl hydroperoxide (tBHP) was used as a positive control in the experiment (FIG. 102).

### 2. Inhibiting Hemichannels Protects Against Radiation-Induced Cell Death

In order to investigate cell death after IR exposure, Annexin V (early and late apoptotic cells) and Caspase 3/7 activity (late apoptotic cells) were assessed in TICAE and TIME cells from 4 h until 100 h after IR using IncuCyte live cell imaging. We furthermore tested the effect of hemichannel inhibition with TAT-Gap19.

In TICAE cells, a significant increase in Caspase 3/7 activity and Annexin V was observed for the 5 Gy dose starting from 4 h and persistent until 100 h p.i., the last point of the recording (FIG. 103 and FIG. 104. TAT-Gap19 significantly reduced Caspase 3/7 and Annexin V for the 5 Gy dose starting from 12 h p.i. and persistent until the end of the recording (FIG. 103 and FIG. 104. In addition, TAT-Gap19 significantly reduced Caspase 3/7 activity and Annexin V for 0 Gy and 0.1 Gy conditions starting from different time points (4-16 h p.i.) and persisting until the end (FIG. 103 and FIG. 104.

In TIME cells, there was no significant change in Caspase 3/7 activity after 0.1 Gy and 5 Gy of IR until 100 h p.i. (FIG. 149). However, a significant persistent increase in Annexin V level was observed at 5 Gy starting from 4 h p.i. until the end of the recording (FIG. 105). TAT-Gap19 significantly reduced Annexin V elevation for the 5 Gy dose in the 4-48 h p.i. time window. TAT-Gap19 also transiently reduced Annexin V for the 0 Gy condition (12-24 h p.i. time window;

FIG. 105).

To further validate cell death in TIME cells, 10 kDa dextran fluorescein dye, which is known to enter and stain cells during necrosis or late apoptosis, was assessed at 6 h and 72 h p.i. A radiation-induced increase in dextran fluorescein was observed for 5 Gy at 6 h p.i. and for both 5 and 0.1 Gy doses at 72 h p.i. (FIG. 106 and FIG. 107). These responses (including the control response) were inhibited by TAT-Gap19 (FIG. 106 and FIG. 107). An unexpected small increase in dextran fluorescein staining was observed with TAT-Gap19 for the control and 0.1 Gy conditions at 6 h p.i. (FIG. 106 and FIG. 107).

### 3. Inhibiting Hemichannels Mitigates Radiation-Induced Inflammatory Responses

Different atherosclerosis inflammatory markers, selected based on a survey of available literature, were assessed in TICAE and TIME cells after 0.1 Gy and 5 Gy of IR exposure at 24 h, 48 h, 72 h and 7 d p.i. We subsequently tested whether hemichannel inhibition with TAT-Gap19 affected these responses. The various inflammatory markers used are summarized in FIG. 150. In total, we considered 12 different markers.

In general, IR exposure at a 5 Gy dose induced an increase in the majority of the markers (IL-6, MCP-1, PECAM-1, IL-1B, TNF-α, CRP, VCAM-1, E-selectin, Endothelin-1, IL-8 and PAI-1) in both TICAE and TIME cells. TAT-Gap19 displayed variable effects, in some conditions decreasing while in others increasing the responses, especially in TICAE cells (FIG. 150). In order to test the possibility that the viral TAT membrane translocation sequence could perhaps by itself elicit an effect, we selected several markers and evaluated their response to TAT peptide exposure at the 24 h time point, that corresponds to the time at which TAT-Gap 19 increased all tested markers in TICAE cells. We found that TAT (100 μM) significantly increased IL-6, MCP-1, IL-1B, CRP and Endothelin-1 in TICAE cells (FIG. 151 to FIG. 155). Notwithstanding these early (24 h) TAT side-effects on a subset of the markers in TICAE cells, TAT-Gap19 significantly reduced the responses of several markers in TICAE and TIME cells mostly at later time points. FIG. 108 to FIG. 131 summarize these responses for selected markers and time points, which are considered in the account that follows. In TICAE cells, MCP-1, VCAM-1 and IL-8 were significantly increased after 5 Gy IR exposure at 72 h and 7 d p.i., and TAT-Gap 19 significantly decreased these responses (FIG. 108-FIG. 109, FIG. 110-FIG. 111 and FIG. 112-FIG. 113). Radiation induced an increase in Endothelin-1 and IL-11 at 7 d p.i. in TICAE cells, and TAT-Gap 19 again significantly reduced these responses (FIG. 114 & FIG. 115).

In TIME cells, a radiation-induced significant increase in IL-6 at 5 Gy was observed at 24 h, 48 h and 7 d p.i., and TAT-Gap19 significantly reduced these responses (FIG. 116, FIG. 117 & FIG. 118). Next to that, radiation induced a significant increase in VCAM-1 for 5 Gy at 24 h and 7 d p.i. in TIME cells, and TAT-Gap19 again significantly reduced these responses (FIG. 119 & FIG. 120). Likewise, Endothelin-1 was significantly increased after 5 Gy irradiation in TIME cells at 24 h, 72 h and 7 d p.i., and TAT-Gap19 significantly decreased these responses (FIG. 121, FIG. 122 & FIG. 123). Furthermore, TAT-Gap19 significantly reduced the increase of TNF-α, E-Selectin, PECAM-1, MCP-1, CRP, IL-1B and ICAM-1 at 24 h after 5 Gy irradiation in TIME cells (FIG. 124, FIG. 125, FIG. 126, FIG. 127, FIG. 128, FIG. 129 & FIG. 130). TAT-Gap19 also significantly decreased IL-8 levels after 5 Gy IR exposure at 7 d p.i. (FIG. 131) in TIME cells. In some of the experiments described above, TAT-Gap19 also had effects on the 0 Gy control and 0.1 Gy conditions in both cell types: it increased the MCP-1 control readout as well as the 0.1 Gy at 72 h p.i. measurement in TICAE cells (FIG. 108), and also the IL-6 control and 0.1 Gy at 7 d p.i. readouts in TIME cells (FIG. 118). By contrast, it decreased the control and the 0.1 Gy readouts for Endothelin-1 in TICAE and TIME cells (FIG. 114, FIG. 122 & FIG. 123), and the 0.1 Gy readouts for, IL-6, VCAM-1, PECAM-1, TNF-α, E-selectin and IL-8 in TIME cells (FIG. 114, FIG. 116, FIG. 120, FIG. 122, FIG. 123, FIG. 124, FIG. 126, FIG. 128 & FIG. 131), suggesting baseline/low dose effect of TAT-Gap19.

I. Inhibiting hemichannels protects from radiation-induced premature endothelial senescence

In order to assess premature senescence in TICAE and TIME cells after 0.1 Gy and 5 Gy irradiation and to evaluate the effect of blocking Cx43 hemichannels, senescence-associated β-galactosidase (SA β-gal) activity was measured at 7, 9 and 14 d after exposure.

In TICAE cells, a radiation-induced increase in SA β-gal activity was observed for 5 Gy at 7 and 9 d p.i.. By contrast, at the 0.1 Gy 7 d p.i. time point, SA β-gal activity was significantly decreased (FIG. 132 and FIG. 133). TAT-Gap19 significantly reduced SA β-gal activity at control, 0.1 Gy and 5 Gy conditions for 7 d and at 0 Gy and 5 Gy irradiated conditions for 9 d p.i. (FIG. 132 and FIG. 133). In TIME cells, a radiation-induced increase in SA β-gal activity was observed for 5 Gy at 7 d p.i. and for 0.1 Gy at 9 d p.i. (FIG. 135 and FIG. 136). TAT-Gap19 significantly reduced SA β-gal activity in all conditions (0 Gy, 0.1 Gy and 5 Gy) at the 7 d p.i. time point and at the 9 d p.i. time point for 5 Gy; although it unexpectedly increased SA β-gal activity in the control condition in TIME cells at 9 d p.i. (FIG. 135 and FIG. 136). At 14 d p.i., SA β-gal activity was decreased after 5 Gy exposure but significantly increased after 0.1 Gy exposure in both TICAE and TIME cells, and TAT-Gap19 significantly inhibited these responses (FIG. 134 and FIG. 137).

In order to further assess premature senescence, levels of the Insulin-like Growth Factor-binding Protein-7 (IGFBP-7) and GDF15 growth differentiation factor 15 (GDF-15), known to be involved in senescence, were assessed at 7 d after IR exposure using multiplex-based assays. A radiation-induced increase in IGFBP-7 was observed at 5 Gy for TICAE and TIME cells, but this response was not inhibited by TAT-Gap19. By contrast, 0.1 Gy did not significantly increase IGFBP-7 at 7 d p.i. but TAT-Gap19 acted in a significantly inhibitory way in this condition (FIG. 138 and FIG. 140). The control readout in TIME cells was increased by TAT-Gap19 (FIG. 140). GDF-15 appeared more sensitive to irradiation and was significantly increased in TICAE and TIME cells at both 0.1 and 5 Gy doses at 7 d p.i. exposure. TAT-Gap19 significantly inhibited these responses in all treated conditions including the 0 Gy condition (FIG. 139 and FIG. 141).

### 2. Radiation-Induced DNA Damage is not Affected by Hemichannel Inhibition

DNA damage was assessed by immunocytochemical staining for gamma H2AX and TP53BP1, DNA double strand break markers, in TICAE and TIME cells after 1 h of X-ray exposure (0.1 Gy and 5 Gy). We furthermore tested the effect of hemichannel inhibition with TAT-Gap19.

In TICAE and TIME cells, radiation induced a significant dose-dependent increase in gamma H2AX and TP53BP1 foci at the 5 Gy dose (FIG. 142, FIG. 143, FIG. 145, FIG. 146 and FIG. 148). The colocalized gamma H2AX/TP53BPI foci also significantly increased in a dose-dependent manner in the irradiated TICAE and TIME cells (FIG. 144, FIG. 147 and FIG. 148). These responses were not affected by TAT-Gap19.

### DISCUSSION

Patients treated with radiotherapy have an increased risk of cardiovascular disease, but the underlying pathophysiology is complex and not fully understood. Here, we aimed to investigate the role of Cx43-based hemichannels in radiation-induced endothelial cell damage, an early marker of atherosclerosis, making use of the peptide inhibitor TAT-Gap19. We found that this peptide significantly reduced oxidative stress, cell death, key inflammatory cytokines and premature cell senescence induced by X-ray exposure of endothelial cell lines derived from coronary artery and microvascular endothelial cells. Below we discuss these findings in more detail.

A predominant effect of IR exposure is ROS production and oxidative stress. Accordingly, in TICAE (coronary artery-derived) and TIME cells (microvascular endothelial cells TIME), we found a dose-dependent increase in oxidative stress after 0.1 Gy and 5 Gy of X-ray exposure as measured by CM-H2DCFDA-based flow cytometric and live cell imaging approaches. The highest oxidative stress response was observed at 45 min p.i. in both cell types, followed by a decline to the 2 and 3 h time points; the latter were however still significantly above non-irradiated controls. The decline at 2-3 h may be linked to antioxidant defence reactions of enzymes such as superoxide dismutases (SOD), glutathione, glutathione peroxidases, catalase and others. A similar time line has been observed in primary microvascular endothelial cells exposed to gamma-rays. Interestingly, we found that blocking Cx43 hemichannels with TAT-Gap19 significantly reduced ROS production in both cell types at 45 min p.i. (FIG. 98 and FIG. 99). Cx43 hemichannel opening has been demonstrated previously in osteocytes in response to oxidative stress, which involved changes of intracellular Ca²⁺ concentration. Oxidative stress was also reported to induce Cx43 hemichannel opening in fibroblastoid rat mammary tumor cell line (Marshall cells) which express Cx43 only. Further downstream, hemichannel opening has been linked to an extracellular depletion of the major antioxidant glutathione, thereby exaggerating oxidative stress in renal tubular epithelial cells. Along the same line, Cx43 hemichannel inhibition with TAT-Gap 19 increased SOD activity in mice liver treated with thioacetamide to induce liver fibrosis (66). We hypothesize that IR exposure induces ROS production in endothelial cells, which opens Cx43 hemichannels and leads to either loss of hemichannel-permeable antioxidants such as glutathione or activates other signalling pathways that lead to the loss of larger antioxidants such as SOD. TAT-Gap19 attenuated this oxidative stress response, but given the important bidirectional relation between [Ca²⁺], elevation and ROS generation, it is also possible that hemichannel inhibition directly reduces ROS production by inhibiting Ca²⁺ entry through hemichannels.

It is well known that oxidative stress is associated with the activation of various cascades including the DNA damage response, apoptosis and inflammatory pathways, thereby amplifying endothelial dysfunction. We observed radiation-induced DNA damage in the two cell types at 1 h p.i., as evidenced from gamma-H2AX and TP53BP1 foci analysis. These findings are consistent with previous studies where a dose-dependent increase in DNA damage was observed in EA.hy926 endothelial hybrid cells, in umbilical vein endothelial cells, as well as in coronary artery endothelial cells in response to X-ray doses in the range of 0.05 to 2 Gy, and in microvascular endothelial cells after 3 and 10 Gy of gamma-rays. Our findings showed that TAT-Gap19 did not alter this DNA damage response in the irradiated TICAE and TIME cells.

We found that cell death in the two cell types mainly occurred after 5 Gy irradiation, with TICAE cells responding with Annexin V and Caspase 3/7 elevation while TIME cells responded with Annexin V elevation and membrane leakage as indicated by cellular dextran fluorescein positivity. The absence of Caspase 3/7 elevation in TIME cells confirms the findings in primary human microvascular endothelial cells exposed to X-rays in the 2 to 12 Gy dose range. Overall, the cell death observed in our study is in line with previous findings in umbilical vein endothelial cells and microvascular endothelial cells. Interestingly, we show that TAT-Gap19 significantly reduced cell death in irradiated TICAE and TIME cells. Cx43 hemichannels are known to mediate cell death in various model systems as well as in cardiomyocytes exposed to hypoxia-reoxygenation or ischemia-reperfusion, by facilitating the passage of soluble factors and molecules such as ATP, ROS and Ca²⁺. It was reported that IR induces hemichannel-related ATP release which may cause intracellular ATP depletion, thereby activating cell death process through necrosis or apoptosis. Hemichannel opening has furthermore been suggested to facilitate ROS entry into the cells eventually leading to cell death.

Inflammation plays a key role in atherosclerosis development and progression. In this study, we showed that radiation induced an increase in atherogenic inflammatory markers (IL-6, MCP-1, PECAM-1, IL-1B, TNF-α, CRP, VCAM-1, E-selectin, endothelin-1, IL-8 and PAI-1) mainly at 5 Gy, in both TICAE and TIME cells. Observations in various endothelial cells confirm an elevation of these inflammatory markers mainly at higher irradiation doses (>2 Gy). We found that TAT-Gap19 significantly reduced the elevated IL-1B, IL-8, VCAM-1, MCP-1 and endothelin-1 levels in the two cell lines, at late time points (72 h and 7 d), while it also had early (24 h) inhibitory effects in TIME cells. In addition, TAT-Gap19 displayed early (24 h) inhibition of TNF-α, E-Selectin, PECAM-1, CRP and ICAM-1 but only in TIME cells, indicating that Cx43 hemichannel involvement in the inflammatory response is time and cell type dependent. All cytokines listed are known to be strongly involved in the pathogenesis of radiation-induced atherosclerosis. The cytokine response is triggered by IR/oxidative stress-induced NF-κB signaling and results in endothelial cell activation with subsequent expression of adhesion molecules. TNF-α was shown to induce smooth muscle cell proliferation and to increase monocyte adherence to endothelial cells by inducing the expression of cell adhesion molecules. Expression of adhesion molecules, such as VCAM-1, ICAM-1, PECAM-1 and E-selectin, on the vascular wall is known to initiate the recruitment of macrophages and leukocyte from the vasculature, that acts together with MCP-1 and IL-8 to induce infiltration of macrophages into the subendothelial cell layer, leading to the formation of atherosclerotic lesions. IL-6 has also been shown to be responsible for propagating downstream inflammatory responses, which involve lipoprotein oxidation by phospholipases, recruitment of other pro-inflammatory cytokines and the release of prothrombotic mediators, that contribute to atherosclerotic plaque development and plaque destabilisation. Moreover, an increased production of the potent vasoconstrictor peptide Endothelin-1 was linked to endothelial cell dysfunction, as it may decrease endothelial nitric oxide synthase (eNOS) expression, thereby reducing nitric oxide (NO) vasodilatory signaling, impairing the regulation of vascular tone. Endothelin-1 was also reported to activate macrophages, thereby supplementing the pro-inflammatory and chemotactic scene with TNF-α, IL-6, IL-8 and others, which are involved in the pathogenesis of atherosclerosis. Importantly, it is thought that Cx43 hemichannel opening may continue and propagate the inflammatory scene. In addition, upregulated Cx43 expression often correlates with increased inflammatory responses, while reduced Cx43 expression inhibits inflammation. TAT-Gap19 has been demonstrated to significantly decrease IL-1B and TNF-α in non-alcoholic fatty liver mouse model. Peptide5, another mimetic peptide-based hemichannel blocker was reported to reduce TNF-α and IL-1B in rat model of spinal cord injury. This peptide also reduced inflammation in a rat model of light-induced retinal damage, as assessed by glial fibrillary acidic protein (GFAP) and leukocyte common antigen (CD45) immunohistochemistry. A possible mechanism by which Cx43 hemichannels contribute to inflammation is through their regulatory effect on high-mobility group box 1 (HMGB1), which serve as a damage-associated molecular pattern molecule (DAMP). HMGB1, released by apoptotic cells, induces IL6, IL-8 and MCP-1 secretion and increases the expression of ICAM-1 and VCAM-1 in endothelial cells. Furthermore, by releasing ATP, Cx43 hemichannels enhance inflammation through downstream activation of the NLRP3 inflammasome. Cx43 hemihannels may thus act to amplify and perpetuate an initial inflammatory condition. Interestingly, IR induces a rapid and persisting increase in Cx43 gene and protein expression in TICAE and TIME cells, thereby creating pro-inflammatory substrate.

In contrast to the inflammatory mitigating effect of TAT-Gap19 on irradiated endothelial cells, the peptide transiently increased some other inflammatory markers at various time points (FIG. 150). TAT-Gap19 is composed of the nonapeptide Gap19, linked to a HW-derived TAT sequence to promote its membrane permeability. It is possible that this HIV-derived membrane translocation sequence may induce pro-inflammatory side effects in the cells. In line with this, alpha-carboxy terminus 1-peptide (aCT1), another Cx43 based peptide containing the antennapedia translocation motif, triggered a transient increase in TNF-α upon topical corneal application. Here, we report that the TAT translocation motif by itself induces a transient increase in IL-6, MCP-1, IL-1B and CRP in TICAE cells at 24 h p.i. (the time point at which TAT-Gap19 significantly increased all assessed cytokines in TICAE cells). Taken this into account, our results indicate that TAT-Gap19 exerts a late inflammatory mitigating effect, by reducing the atherogenic inflammatory markers IL-1(3, IL-8, VCAM-1, MCP-1 and Endothelin-1, in both TICAE and TIME cells after IR exposure.

Interestingly, inflammation has been reported to contribute to cellular senescence. Premature cell senescence is known to contribute to endothelial dysfunction by stimulation a complex pro-inflammatory response, including the release of adhesion molecules such as IL-6, IL-8. MCP-1 and IGFBP, and it is known to trigger the initiation of cell death by reducing cell repair ability. Furthermore, endothelial senescent cells are known to increase levels of ROS partly due to downregulation of eNOS leading to mitochondria dysfunction. Our experiments showed an increase in SA-β gal activity in TICAE and TIME cells mainly at 7 and 9 d p.i. This increase in SA-β gal activity was persistent until 14 d p.i. only at 0.1 Gy. For 5 Gy, an unexpected decrease in SA-β gal activity was observed, which may be caused by the frequent medium change for this time point, resulting in washout of senescent cells and replacement by proliferating cells at the high dose. Premature senescence in TICAE and TIME cells was also apparent from the increase in IGFBP-7 (at 5 Gy) and GDF-15 (at 0.1 and 5 Gy) 7 d p.i. GDF-15 is suggested to contribute to premature cell senescence induced by irradiation via the ROS-mediated p16 pathway, while IGFBP-7 is suggested to induce senescence via inhibition of BRAF-MEK-ERK signalling. Additionally, GDF-15 was reported to be involved in the pathogenesis of atherosclerosis by regulating IL-6 and macrophage chemotaxis. Our results are in line with previous studies, where cellular senescence was observed after low and high doses of IR exposure in different types of endothelial cells, including human coronary artery. Interestingly, TAT-Gap19 significantly suppressed the radiation-induced increases in SA-0 gal activity and GDF-15 levels in TICAE and TIME cells suggesting a role for Cx43 hemichannels. Senescence can be communicated to neighbouring cells in a paracrine manner involving secretion of senescence-associated secretory phenotype (SASP) and inflammasome activation. Presumably, Cx43 hemichannels may perhaps facilitate paracrine senescence communication between cells via its effects on pro-inflammatory parameters like IL-8, MCP-1, which are inhibited by TAT-Gap19 in our study.

In summary, this study demonstrates that Cx43 hemichannels play a role in radiation-induced endothelial oxidative stress, cell death, pro-inflammatory and pathological factors like IL-1B, IL-8, VCAM-1, MCP-1 and endothelin-1 and premature senescence. Further research is needed to investigate the underlying pathways and molecular mechanisms involved in Cx43 hemichannel opening in response to radiation exposure. This may open perspectives for the establishment of novel therapeutic strategies to protect from secondary radiotherapy side effects in thoracic cancer patients.

### Conclusion

In conclusion, to the best of our knowledge, we show for the first time the involvement of endothelial Cx43 hemichannels in contributing to various radiation-induced processes, such as oxidative stress, cell death, inflammation and premature cell senescence that lead to endothelial activation and dysfunction, which is known as an early marker for atherosclerosis. Therefore, targeting Cx43 hemichannels with an inhibiting agent promises to therapeutically protect against radiation-induced endothelial cell damage.

### Example 2: Cx43 Channels and Ca²⁺/ROS/NO Signaling Contribute to Radiation-Induced Bystander Effects in Brain Microvascular Endothelial Cells

### Materials and Methods

### 1.1. Cell Cultures

The RBE4 (Rat Brain Endothelial) cell line was kindly provided by Dr F. Roux (Neurotech, Evry, France). The RBE4 cells were grown on collagen (rat-tail collagen; Roche diagnostics, Vilvoorde, Belgium) coated recipients in alfa-MEM+F10 (1/1) medium supplemented with 0.6% geneticin, 1% L-glutamax, 10% fetal bovine serum (FBS, Gibco, Invitrogen, Merelbeke, Belgium) and 1 ng/mL human recombinant basic fibroblast growth factor (hbFGF, Roche diagnostics). Cells plated for the radiation experiments were grown without hbFGF. Next to this cell line, also primary brain microvascular endothelial cells (pBMECs) isolated from C57BL6 and C57BL/6 Cx43^{fl/fl}:Tie2-Cre mice, were used. Primary BMECs were grown in DMEM (Gibco, Invitrogen, Merelbeke, Belgium) supplemented with 20% Newborn Calf Serum (PAN Biotech, Aidenbach, Germany), 1% glutamax (Gibco, Invitrogen, Merelbeke, Belgium), 0.5% gentamicin (Gibco, Invitrogen, Merelbeke, Belgium), 1% vitamins, 2% amino acids and 1 ng/mL hbFGF; hbFGF was removed from the cultures 24 h prior to irradiation. Primary BMECs cultures were grown on plates coated with matrigel (3.5 μg/cm²). Patch clamp experiments were performed on HeLa cells stably transfected with Cx43, were cultured in Dulbecco's Modified Eagle's Medium (Invitrogen, Ghent, Belgium), supplemented with 10% FBS, 2 mM glutamine, 10 μg/ml streptomycin, 10 U/ml penicillin, 0.25 μg/ml fungizone (Invitrogen, Ghent, Belgium) and 1 μg/ml puromycin (Sigma-Aldrich, Bornem, Belgium). Hela-WT cells were grown in the medium without puromycin. Mouse Cx43 gene was cloned into the EcoRI/BamHI restricted cloning site of the expression vector pMJgreen. CytoMegalovirus (CMV) promoter was used. The vector also contains a puromycin N-acetyl-transferase (Pac) gene encoding region.

### 1.2. Agents

2′,6′-diamidino-2-phenylindole (DAPI), carbenoxolone (Cbx), N-acetyl-L-cystein (NALC), Pyridoxalphosphate-6-azopehyl-2′,4′-disolfonic acid (PPADS) and 4% formaldehyde were purchased from sigma-aldrich. 1.2-bis-(2-aminophynoxy)-ethane-N,N,N′,N′,tetraacetic acid acetoxy methyl ester (BAPTA-AM) originates from molecular probes (Invitrogen, Merelbeke, Belgium). Following connexin mimetic peptides were used: Gap26 (amino acids 64-76 from the first extracellular loop of Cx43, the sequence of which is shown in SEQ ID NO: 1) and TAT-gap19 (amino-acids 128-136 in the second half of the CL of Cx43, the sequence of which is shown in SEQ ID NO: 2) obtained from Genosphere biotechnologies (Genosphere biotechnologies, Paris, France) with a purity >85%.

### 1.3. Isolation of Brain Microvascular Endothelial Cells

Cortices, from 10-12 week old mice, were isolated by removing cerebellum, striatum, optic nerves, brain white matter, outer vessels and meninges. After homogenization with a Dounce homogenizer in WBB (Washing Buffer B: HMS, 10 mM Hepes, 0.35 g/L NaHCO₃, 0.1% BSA), the homogenate was mixed with 30% dextran in WBB and centrifuged at 3000×g for 25 min at 4° C. The pellet containing the vascular component was then resuspended in WBB and filtered through a 60 μm NY60 Nylon Net Filter (Millipore, Darmstadt, Germany). Following centrifugation at 1000×g for 7 min at room temperature, the pellet was digested in collagenase/dispase supplement with DNase I (Roche Diagnostics, Vilvoorde, Belgium) and TLCK (Tosyl, Lysin chloromethyl ketone, sigma-aldrich) for 33 min at 37° C. in a shaking water bath. The digested capillary suspension was then seeded after multiple washing steps on wells coated with matrigel or glass coated with Corning Cell-Tak (VWR, Leuven, Belgium). For the isolation of intact capillaries the protocol was aborted just before the digestion step.

### 1.4. X-Ray Irradiation

A defined area of the cell culture dishes were exposed to X-rays (1Gy or 20 Gy) by using a small animal radiation research platform (SARRP, Xstrahl, 220 kV, 13 mA), making use of a 3×3 mm collimator. Whole dish irradiation was done using a 10×10 cm broad-beam collimator, while localized Irradiation was performed with a 3×3 mm collimator. A Gafchromic RTQA2 film, with a sensitivity of 0.02 Gy, was placed underneath the cell dishes in order to delineate the irradiated zone. Control experiments with cells exposed to 0.02 Gy did not produce any detectable effect on the γ-H2AX scores (data not shown), excluding the possibility that scattered irradiation not detected by the irradiated film would influence the results in the bystander area.

### 1.5. γ-H2AX Immunostaining and Counting Procedure

Cells were fixed for 25 min with 4% formaldehyde (VWR) and blocked for 30 min with blocking buffer (5% normal goat serum, 1% bovine serum albumin (BSA), 0.2% Triton X-100 in PBS D+). Overnight incubation with primary antibody (1/500 anti-γ-H2AX in dilution buffer, 1/10 blocking buffer in PBS D+) was followed by a 1 h incubation with 1/400 XX-biotin-goat-anti-mouse antibody in dilution buffer, followed by a 1 h incubation with 1/400 streptavidin-alexa488 in PBS D+. Nuclei were stained with 1 μg/mL DAPI in PBS D+ for 10 min and cells were kept in PBS D+ supplemented with NaN₃ at 4° C. All steps except the overnight incubation which was carried out at 4° C., were carried out at room temperature and cell cultures were rinsed thoroughly with PBS D+ between all incubation steps. Imaging was performed with an automated BD Pathway 435 imaging system (10× objective, 10×10 montage resulting in an overall image size of 8.5×6.5 mm). We then quantified the number of γ-H2AX-foci positive nuclei in the directly irradiated and bystander areas and expressed the count as a percentage relative to the number of nuclei and subtracted the percentage of background γ-H2AX signal in non-irradiated paired control cultures for each experiment. For counting γ-H2AX foci, images were thresholded to remove background pixel noise below the foci level. Where applicable, results were normalized against vehicle (bar charts with a 100% vehicle bar without statistical variability or a horizontal line).

We tested how counting γ-H2AX positive nuclei over a large surface area (9 and 55 mm² for irradiated and non-irradiated zones respectively) was related to the more classical cell-based approach of quantifying the number of γ-H2AX foci per nucleus. To that purpose, we acquired high magnification (×63 objective) images and quantified the relative area occupied by γ-H2AX foci per nucleus in the irradiated zone. We found a linear relation between the low (×10 objective) and high magnification-based quantifications in the range of 0.1 to 1 Gy; at higher 10-20 Gy doses, the relation flattened (FIG. 227 to FIG. 229). In the bystander zone, the fraction of γ-H2AX positive nuclei was in the range of 4-12%, which fell in the linear range.

Gamma-H2AX foci counts of FIG. 167 & FIG. 168 were calculated in a different way: for FIG. 167, we calculated the summed raw counts of all foci-positive vertical pixel columns for each pixel row position along the image x-axis. For FIG. 168, counting was done as explained for FIG. 167 but normalized to the counts at the border of the irradiation zone (100%).

All γ-H2AX foci images (FIG. 156 to FIG. 158, FIG. 160 to FIG. 164, FIG. 166, FIG. 173 and FIG. 174) are raw unprocessed immunofluorescence images; the foci images of FIG. 218 and FIG. 219 were processed by a digital dilation operation by using ImageJ software to increase the size of individual foci-positive dots in order to increase visibility of the staining at these higher magnification images.

### 1.6. Gel Electrophoresis and Western Blotting

RBE4 cells or pBMECs were seeded in 25 cm² falcons or 8 cm² petridishes. Lysates were made with RIPA (Cx43 and Cx37) and laemmli (Cx40) buffer. Protein concentration was determined using the BioRad DC protein assay kit (BioRad, Nazareth, Belgium). The lysate was separated by SDS-PAGE, over a mini-protean TGX stain free gel (BioRad, Nazareth, Belgium) and transferred to a nitrocellulose membrane (Amersham, Buckinghamshire, UK). Membranes were blocked in TBS supplemented with 5% (Cx43 and Cx40) or 2% non-fat milk (Cx37) and 1% (Cx43 and Cx40) or 0.05% (Cx37) Tween20. The following primary antibodies were used: rabbit-anti-Cx43 (sigma), rabbit-anti-Cx37 (anti-rat and anti-mouse), goat-anti-Cx40 (Santa Cruz) or rabbit-anti-β-tubulin antibody (Abcam, Cambridge, UK). Membranes were subsequently incubated with an alkaline phosphatase-conjugated goat anti-rabbit (Cx43, Cx37) or donkey anti-goat (Cx40) IgG antibody (sigma-aldrich). Detection was done using the nitro-blue-tetrazolium/5-bromo-4-chloro-3-indolyl-phosphate reagent (NBT/BCIP kit, Zymed, Invitrogen). Total protein staining was carried out with SYPRO Ruby protein blot dye (Invitrogen, Molecular Probes, Merelbeke, Belgium).

### 1.7. Immunocytochemistry

For Cx immunoscytochemistry (Cx37, Cx40 and Cx37), fixation was as described for γ-H2AX immunostaining, followed by a 30 min (cell cultures) or 1 h (capillaries) blocking step with blocking buffer B (0.2% Tx100, 0.4% gelatin). Cells were incubated overnight with sheep anti-Cx37 (1/1000, Invitrogen), goat anti-Cx40 (1/50, Santa-Cruz) or rabbit anti-Cx43 (1/500), sigma) combined with rat anti-CD31 (combination of two antibodies, each 1/100, BD and Invitrogen) at 4° C. In a next step, secondary antibodies were administered for 1 h (donkey anti-sheep alexa 594, chicken anti-goat alexa 594, goat anti-rabbit alexa 594 and goat anti-rat alexa 488 respectively, each 1/400 in blocking buffer B).Confocal images were taken with a Leica SP8 X confocal microscope (×63 water immersion objective) and analyzed using Fiji software.

### 1.8. ATP Measurements

ATP was measured using a luciferin-luciferase assay kit (Sigma-Aldrich) in combination with a luminometer plate reader (Victor3 1420 multi label counter; Perkin Elmer, Zaventem, Belgium).

Extracellular ATP measurements were initially carried out at different time-points post-irradiation, the effect of blockers on ATP release was tested at the 5 min post-irradiation time-point (time of maximum ATP release).

### 1.9. Dye Uptake

Cells were grown to near confluency in 4-well plates. Cultures were incubated with 1 mM propidium iodide (PI) and 200 μM 10 kDa dextran-FITC before focal irradiation and dye uptake was measured 5 min post-irradiation by measuring the fluorescence intensity of both dyes following image acquisition with a BD pathway 435 imaging system.

### 1.10. Scrape Loading and Dye Transfer

Confluent monolayer cultures were washed three times with nominally calcium-free scrape loading and dye transfer (SLDT) buffer (137 mM NaCl, 5.36 mM KCl, 0.81 mM MgCl₂.6H₂O, 5.55 mM D-glucose, 25 mM Hepes, pH 7.4). Cells were incubated for 1 min in SLDT buffer containing 400 μM 6-carboxyfluorescein; a linear scratch (one per culture) was made across the cell layer using a syringe needle, and the cells were left for another minute in the same solution. Cultures were then washed six times with HBSS-HEPES and left for 15 min at room temperature, and images were taken as described for electroporation loading.

### 1.11. Electrophysiological Recording

Subconfluent cultures of HeLa-Cx43 cells were seeded on 13 mm diameter glass coverslips (Knittel Glaser, Novolab, Geraardsbergen, Belgium) and experiments were performed at subconfluency the next day. Recordings were performed in the presence of extracellular Ca²⁺ and Mg²⁺ and under conditions of K⁺-channel blockade with Cs⁺, Ba²⁺ and TEA⁺. Cells were bathed in a recording chamber filled with a modified Krebs-Ringer solution, consisting of (in mM): 150 NaCl, 6 CsCl, 2 MgCl₂, 2 CaCl₂), 5 glucose, 5 HEPES, 1 BaCl2 and 2 pyruvate, with pH adjusted to 7.4. The whole-cell recording pipette solution was composed of (in mM): 130 CsCl, 10 NaAsp, 0.26 CaCl₂), 5 HEPES, 2 EGTA, 5 TEA-Cl and 1 MgCl₂, with an adjusted pH of 7.2. Free intracellular Ca²⁺ was 50 nM, as calculated with Webmax Standard software application (http://www.stanford.edu/˜cpatton/webmaxcS.htm). An EPC 7 PLUS patch clamp amplifier (HEKA Elektronik, Lambrecht/Pfalz, Germany) was used to perform single channel recordings. Data were acquired at 6 kHz using a NI USB-6221 data acquisition device from National Instruments (Austin, Tex., USA) and WinWCP acquisition software (designed by Dr. J. Dempster; University of Strathclyde, UK). All currents in whole-cell configuration were filtered at 1 kHz (7-pole Besselfilter). For single channel analysis, holding currents were subtracted from the recorded current traces, giving traces that only contained unitary current events. Unitary conductances were calculated from the elementary current transitions Δi as: γ=Δi/Vₘ. From these data, we constructed all-point conductance histograms that displayed one or more Gaussian distributions. These were fit by a probability density function assuming independent channel opening. Channel activity was quantified from the charge transfer Qₘ associated with unitary currents; this was done by integrating the unitary current traces (i.e., a function of time) over the duration of the voltage step as: Qₘ=∫idt.

### 1.12. Calcium Imaging

Cells were seeded on glass coverslips (18 mm 0) coated with 3.5 μg/cm² Corning cell-tak (VWR, Leuven, Belgium) and ester loaded for 45 min with 10 μM Fluo-3-AM in HBSS-HEPES supplemented with 1 mM of probenecid and 0.01% of pluronic F127 at room temperature followed by de-esterification over 15 min. Imaging was performed using an inverted fluorescence microscope equipped with a ×40 oil immersion objective and an EM-CCD camera (QuantEM™ 512SC CCD camera, Photometrics, Tucson, Ariz.). For direct Ca²⁺ measurements the loaded cells were irradiated and transferred to the microscopy stage. For medium transfer, the loaded cells were superfused for 1 min with HBSS-Hepes (1 mM), followed by 4 min with HBSS-Hepes conditioned by irradiated cultures. Fluorescence intensity changes in the cells were analyzed with custom-developed FluoFrames software (L. Leybaert, Ghent, Belgium). Ca²⁺ changes were quantified as the area under the curve (AUC) of the Ca²⁺ traces.

### 1.13. ROS Measurements

For ROS measurements, the cells were seeded in 96-well plates coated with collagen and irradiated using broad-beam at different doses. For ROS measurements the cells were loaded with 10 μM of CM-H2DCFDA in HBSS-Hepes. Imaging was performed with a BD Pathway 435 microscope equipped with an automated imaging focus system, avoiding ROS generation associated with long exposures to excitation. Results were corrected for the ROS produced in the irradiated medium alone.

### 1.14. Cytotox Glo Assay

To evaluate the amount of dead cells, RBE4 cells were seeded in 96-well plates coated with collagen and irradiated using broad-beam at different doses. The CytoTox-Glo™ Cytotoxicity assay (Promega, Madison, USA) was used following the manufacturers protocol. The CytoTox-Glo™ Cytotoxicity Assay uses a luminogenic peptide substrate, the AAF-Glo™ Substrate, to measure dead-cell protease activity, which is released from cells that have lost membrane integrity. Measurements were performed with aluminometer plate reader (Victor3 1420 multi label counter; Perkin Elmer, Zaventem, Belgium).

### 1.15. Electroporation

Cells were grown to near confluency on 4-well plates coated with collagen. Cell monolayer cultures were washed three times with HBSS-Hepes 25 mM (pH 7.2-7.4) and subsequently three times with a low conductivity electroporation buffer (4.02 mM KH2PO4, 10.8 mM K2HPO4, 1.0 mM MgCl2, 300 mM sorbitol, 2.0 mM Hepes, pH 7.4). They were placed 400 μm underneath a two-wire Pt-Ir electrode on the microscopic stage and electroporated in the presence of a tiny amount of electroporation solution (10 μl) containing 100 μg/mL SOD, 60 μM BAPTA or 20 μM BH4-Bcl2 combined with 100 μM 10 kDa DTR. Control cultures were electroporated with solution containing only 100 μM DTR vehicle solution. Electroporation was carried out with 50 kHz bipolar pulses applied as trains of 10 pulses of 2 ms duration each and repeated 15 times. The field strength was 100 V peak-to-peak applied over a 500 μm electrode separation distance. After electroporation, cells were thoroughly washed with HBSS-Hepes 25 mM followed by addition of CO2-independent medium (Invitrogen), which was was also present on the cultures during and following irradiation. The irradiated area was chosen away from the electroporation area (1555±1468 μm border-to-border distance; n=15) in order to investigate the effect of the blockers in the bystander area.

### 1.16. Data and Statistical Analysis

Data are expressed as mean±SEM, with ‘n’ denoting the number of independent experiments. Multiple groups were compared by one-way ANOVA and a Bonferroni post-test, using GraphPad Instat Software (Graphpad Software). P<0.05 was considered statistically significant. Statistical significance is indicated in the graphs by one symbol for P<0.05, two symbols for P<0.01 and three symbols for P<0.001.

### Results

1. X-Ray-Induced DNA Damage is Propagated from Irradiated to Non-Irradiated Bystander Cells in Microvascular Brain Endothelial Cells.

To investigate the role of connexin-mediated intercellular communication in radiation-induced bystander responses, we irradiated a defined area of adherent brain microvascular endothelial cell cultures. Both RBE4 cells, a rat brain microvascular endothelial cell line, as well as primary brain microvascular endothelial cells (pBMECs) isolated from mouse brains, grown to confluency, were used to that purpose. These cells express Cx37, Cx40 and Cx43 based on western blotting and immunocytochemical studies (FIG. 222 to FIG. 226). Endothelial cell monolayer culture dishes were locally exposed to X-rays (1 and 20 Gy) using a 3×3 mm collimator (FIG. 159), resulting in an irradiated area of about 9 mm² within a total 190 mm² cell dish surface area. The irradiated zone was identified by radiosensitive Gafchromic RTQA2 film positioned below the cell dish (FIG. 165). After irradiation, γ-H2AX immunostainings were performed and imaged in a 55.25 mm² large area that included the irradiated and bystander zones. Overlays between the Gafchromic film and γ-H2AX staining allowed to distinguish irradiated and surrounding bystander areas (FIG. 166). We quantified the spatial profile of radiation intensity (Gafchromic film-based) and γ-H2AX foci (summed raw counts of all foci-positive vertical pixel columns for each pixel position along the x-axis of the image; FIG. 167). The γ-H2AX graph showed sharp falling edges at the collimator borders, while the radiation intensity profile displayed more smoothly varying slopes. Taking into account these differences, we defined the borders of the irradiated zone based on the full width at two thirds of the maximum radiation intensity (double arrowed line in FIG. 167), delineating a zone of approximately 3.4×3.4 mm (11.6 mm²). As the experiments described further involved transfer of irradiated cell dishes to the lab for subsequent analysis, we verified whether leaving the cells immobile would give different γ-H2AX scores in the bystander zone. We found that movement and associated shaking of the cells dishes (2 mm medium above the cells) gave a different spatial profile determined 30 min after irradiation (FIG. 168), with significantly lower γ-H2AX scores in the bystander zone (quantified as in FIG. 167 but now normalized to the counts at the border of the irradiation zone) compared to the non-shaken condition at the same 30 min time point. This suggests involvement of paracrine bystander communication, with shaking disturbing an unstirred layer thereby diluting locally released bystander signaling molecules.

In a next experiment, we determined the kinetics of the γ-H2AX signal. Here, as in all other experiments discussed further, γ-H2AX quantification was done by counting γ-H2AX-positive nuclei, expressed relative to the total number of nuclei and background corrected by subtracting relative counts from non-irradiated paired control cell dishes (γ-H2AX count expressed as a percentage). Using this relative count, we observed a dose-dependent increase in γ-H2AX scores in the irradiated zone at 1-3 h after irradiation (at a 1 Gy dose, followed by a gradual recovery over the following 24 h time frame (FIG. 169). We also observed increased γ-H2AX scores in the bystander area, which peaked at 3-5 h post-irradiation followed by recovery 24 h later (FIG. 170). Interestingly, γ-H2AX responses in the bystander zone were not different between 1 Gy and 20 Gy irradiation (8±3.8% vs 6.7±2.9% for 1 Gy and 20 Gy doses respectively 3 h post-irradiation; n=5-10) suggesting that bystander responses saturate at doses below 20 Gy, as reported by others. The γ-H2AX scores recorded 3 h post-irradiation in the bystander area were not different in pBMECs compared to RBE4 cells (FIG. 171). We chose the 1 Gy dose for further studies as it was equally potent as 20 Gy to induce bystander effects.

2. Gap Junction and/or Hemichannel Inhibitors Reduce γ-H2AX Scores in the Bystander Area

Paracrine factors and direct cell-cell communication via gap junctions are both critical in mediating bystander effects. As such, not only gap junctions, but also hemichannels, could contribute to the DNA damage propagation process. To investigate the contribution of gap junctions and hemichannels, we tested the effect of several connexin channel inhibitory substances: the general connexin channel blocker carbenoxolone (Cbx), Gap26 peptide that targets Cx43, Cx40 and Cx37 and rapidly inhibits hemichannel-related responses within minutes and gap junctions with longer exposures and TAT-Gap19 peptide, which blocks hemichannels composed of Cx43 with a half-time of 100 s, without inhibiting gap junctions. Cbx (50 μM), applied 30 min before 1 Gy irradiation and also present in the culture medium thereafter (30 min+3 h post-irradiation), significantly inhibited γ-H2AX-positive cell counts in the bystander zone while its effect in the irradiated zone was not statistically significant (RBE4 cultures; FIG. 175 and FIG. 176). By contrast, Gap26 and TAT-Gap19 (161 μM and 200 μM resp.; 30 min+3 h post-irradiation protocol as for Cbx) significantly inhibited γ-H2AX counts in both irradiated and bystander zones of RBE4 cultures. These experiments were repeated on the primary isolated BMECs. Addition of Cbx resulted in a significant decrease of γ-H2AX counts in both the irradiated area and the bystander zone whereas Gap26 and TAT-Gap19 only had effect in the bystander zone (FIG. 177 and FIG. 178). Both Cbx and Gap26 inhibit channels formed by different connexin isotypes, while TAT-Gap19 specifically targets Cx43-based hemichannels. To further substantiate the contribution of Cx43, we isolated pBMECs from C57BL/6 Cx43″:Tie2-Cre mice that have targeted Cx43 knockdown in endothelial and hematopoetic cells under control of the Tie2 promoter. While pBMECs derived from these animals displayed strongly decreased Cx43 expression compared to Cx43″ mice, Cx43 knockdown did not result in a significant decrease of the bystander response 3 h post-irradiation (FIG. 179). However, western blot analysis (FIG. 179 and FIG. 179) demonstrated increased Cx37 expression in pBMECs from C57BL/6 Cx43″:Tie2-Cre mice compared to Cx43″ mice (FIG. 232), indicating compensatory Cx37 upregulation which may mask the effect of Cx43 knockdown.

### 3. X-Rays Induce Cx43 Hemichannel Opening Via an Increase in Intracellular Ca²⁺ and ROS Production

We examined whether hemichannels open in response to X-ray exposure by using three different approaches: (i) measuring ATP-release in the supernatant, (ii) determining the cellular uptake of the hemichannel-permeable dye propidium iodide (PI), and (iii) patch clamp experiments. For ATP release, we irradiated RBE4 cells with a whole field broad-beam collimator and measured ATP 5 min post-irradiation. Both 1 and 20 Gy irradiation resulted in a significant increase in extracellular ATP compared to non-irradiated cultures and gradually returned to baseline non-irradiated levels within a 3 h time period (FIG. 182). ATP release was inhibited by Cbx, Gap26 and TAT-Gap19 (added 30 min before and present until the end; FIG. 183). In line with these results, a significant increase in the uptake (FIG. 185) of the hemichannel-permeable fluorescent dye propidium iodide (PI) was detected in irradiated cultures, both in the irradiated zone and the surrounding bystander area at 5 min post-irradiation (1 Gy; FIG. 186 and FIG. 187). PI uptake was significantly reduced by Gap26 in the irradiated and bystander areas (FIG. 188). There was no increase in the uptake of the hemichannel-impermeable dye 10 kDa dextran-FITC post-irradiation (data not shown), indicating membrane integrity; this was confirmed by a protease release assay that was negative, indicating there was no membrane disruption (FIG. 235). For patch clamp experiments, both HeLa cells stably transfected with Cx43 (HeLaCx43) and HeLaWT cultures were exposed to broad-beam irradiation (1 and 20 Gy) followed by rapid transfer to the electrophysiology setup for single channel recording. Both 1 and 20 Gy doses resulted in increased unitary current events triggered by voltage steps from −30 mV to +70 mV applied to activate hemichannel opening (FIG. 189). All-point histograms demonstrated a unitary conductance of about 220 pS that is typical for Cx43 hemichannels. Potentiation of unitary current activities increased with post-irradiation time (FIG. 190) and averaging the activity over all time points (range 5-87 min) demonstrated significantly increased membrane charge transfer (Qₘ) at 1 Gy and 20 Gy compared to non-irradiated controls (FIG. 191). Of note, scrape loading and dye transfer experiments did not show significant alterations of gap junctional coupling after exposure to a 1 Gy dose (FIG. 233).

4. X-Rays Induce Increases in Intracellular Ca²⁺ and ROS Production that Contribute to hemichannel opening in BMECs

Ionizing radiation has been shown to rapidly generate ROS as well as a rise of cytoplasmic Ca²⁺ ([Ca²⁺]ᵢ) which are two important triggers leading to the opening of hemichannels. We performed live cell [Ca²⁺ ]ᵢ imaging experiments starting 5 min after the exposure of RBE4 cells to broad-beam irradiation. Cells were loaded with the fluorescent Ca²⁺ indicator fluo-3-AM prior to irradiation with 1 or 20 Gy. These experiments demonstrated Ca²⁺ oscillatory activity in the cells, which significantly increased in percentage of oscillating cells and in number of oscillations per cell compared to non-irradiated controls (FIG. 192 to FIG. 195). We also investigated Ca²⁺ responses in bystander cells by using the medium transfer approach. Exposure of fluo-3-AM loaded non-irradiated RBE4 cells to conditioned medium collected from irradiated cultures 5 min post-irradiation resulted in a transient elevation of [Ca²]ᵢ in the recipient cells (FIG. 196-FIG. 199). Addition of Cbx, Gap26 or the ROS scavenger NALC (1 mM) to the irradiated culture (30 min before irradiation) reduced the area under the curve (AUC) of the Ca²⁺ transients in recipient bystander cells (FIG. 200). Addition of Cbx, Gap26 or the purinergic receptor blocker PPADS (50 μM) to the recipient cells, also inhibited the [Ca²]ᵢ responses (FIG. 201).

In a second instance, RBE4 cells were pre-loaded with the oxidative stress marker CM-H2DCFDA-AM, after which the whole cell culture was exposed to X-rays at 1 or 20 Gy. Irradiation induced a significant and dose-dependent increase in signal intensity of the CM-H2DCFDA probe measured 5 min after irradiation (FIG. 202 to FIG. 206).

Finally, a contribution of increases in [Ca²⁺ ]ᵢ and intracellular ROS production on hemichannel opening was investigated by recording the effect of BAPTA-AM (10 μM; 1 h pre-loading) and NALC (1 mM; 30 min pre-loading) on irradiation-induced ATP release. Both compounds significantly decreased ATP release in the supernatant 5 min after 1 Gy irradiation. A similar result was obtained by scavenging NO, a signaling molecule involved in the response to ionizing radiation as well as in hemichannel opening. Preloading of the cells with the NO-scavenger c-PTIO (100 μM; 30 min prior to 1 Gy irradiation) also strongly reduced irradiation-induced ATP release (FIG. 212 and FIG. 213). Taken together, these findings indicate that the opening of hemichannels is an early step in the response of cells to X-rays which involves the Ca²⁺, ROS and NO signaling pathways.

5. Interfering with Ca^{2±}, ROS, NO or ATP Signaling Reduces Irradiation-Induced γ-H2AX Scores in Irradiated and Bystander Zones

To further document the role of Ca²⁺, ROS and NO signaling in the generation of γ-H2AX bystander effects, we applied BAPTA-AM, NALC and c-PTIO before focused irradiation (3×3 collimator) and analyzed their effect on γ-H2AX positive nuclei counts in the irradiated and bystander areas. These blocking reagents significantly reduced the γ-H2AX scores in both areas, most clearly in primary pBMEC cultures (FIG. 210 and FIG. 211) with less clear responses being observed in RBE4 cells (FIG. 208 and FIG. 209), especially in the irradiated area (FIG. 208 and FIG. 210). Interestingly, the IP₃ receptor antagonist Xestospongin C (5 μM) significantly reduced γ-H2AX counts in the bystander zone of RBE4 cells but not in the irradiated area. In line with the ATP release observed in response to irradiation (FIG. 212 and FIG. 213), we found that blocking purinergic receptor signaling with PPADS significantly reduced γ-H2AX scores in the bystander area in RBE4 cultures (FIG. 209).

In addition to effects in the bystander area, some of the pharmacological blockers also had significant effects in the directly irradiated area (FIG. 208 and FIG. 210). As a result, effects in the bystander zone may partly be caused by inhibitory effects in the irradiated zone. We thus set out to determine the effect of interfering with IP₃, Ca²⁺, and ROS signaling in the bystander zone only. To that purpose, we applied in situ electroporation to load a small and defined zone of cells within the bystander area with membrane-impermeable inhibitors of the concerned pathways (FIG. 214 to FIG. 220). To interfere with IP₃ signaling, we loaded the cells with the IP₃ receptor blocking peptide BH4-Bcl-2 (amino acids 6-30 of Bcl-2; MW 3.6 kDa; 20 μM), which interacts with the regulatory/coupling domain of the IP₃R. To interfere with downstream Ca²⁺ signaling, we loaded cell-impermeable BAPTA (MW 628 Da) into the cells. For ROS, we used the high MW scavenger superoxide dismutase (SOD; MW 32.5 kDa; 3 μM). Of all these substances, BAPTA is the only one that can pass through gap junctions and may thus spread beyond the loaded cell zone. To visualize the spatial distribution of the electroporated zone, we used the fluorescent markers 10 kDa dextran Texas red (DTR) and fura red to image the zone for high MW molecules and BAPTA respectively. Immediately after electroporation, the full width at half maximum (FWHM) of the electroporation zone was about 42 μm for the 10 kDa DTR and about 33 μm for fura red (FIG. 217). For DTR, the electroporated zone was not significantly wider 3 h later (about 37 μm). For fura red, the zone was impossible to quantify at 3 h because of low fluorescence intensity; at 1 h we obtained reliable images giving a FWHM of about 45 μm, which is slightly but not significantly larger compared to the width recorded immediately after electroporation. We next decided to quantify the γ-H2AX counts in the electroporated bystander area in the zone visibly indicated by the 10 kDa DTR tracer, which was in all experiments included in the electroporation vehicle solution. The γ-H2AX score within the DTR zone was quantified by the relative background-corrected γ-H2AX score as used for all previous experiments, but now normalized to the score measured in vehicle-electroporated paired experiments performed in parallel. FIG. 220 summarizes average data of these experiments, demonstrating that BH4-Bcl-2, BAPTA and SOD all strongly reduced the γ-H2AX scores in the electroporation-loaded bystander zone. The strongest effects were observed for the IP₃/Ca²⁺ signaling axis (BH4-Bcl-2 and BAPTA), while the SOD effect was less pronounced. In a last step, we investigated the role of IP₃ diffusion via gap junctions, making use of C6 cells stably expressing the V84L mutant Cx26 that is characterized by strongly reduced gap junctional IP₃ permeability. Local collimator-based irradiation of C6-Cx26 V84L mutant cell cultures demonstrated significantly decreased γ-H2AX scores in the irradiated and bystander zones compared to C6-Cx26 WT cells (FIG. 234), indicating that junctional IP₃ diffusion is indeed involved.

### DISCUSSION

The present results demonstrate that bystander communication of DNA damage, assessed by γ-H2AX quantification, involves connexin signaling via gap junctions and hemichannels, the canonical IP₃/Ca²⁺ signaling cascade, extracellular ATP and ROS/NO signaling. Each of these signals have been implicated in radiation-induced bystander signaling, but a coherent framework is currently lacking. Of note, most of these signals play a role in cell-cell communication of Ca²⁺ signals and all of them have been linked to hemichannels, either as a trigger for their opening or as a substance released in a manner facilitated by hemichannels. Specifically, Ca²⁺ signal communication between cells is mediated by gap junctions that pass IP₃ and Ca²⁺ and by paracrine signaling that involves extracellular ATP as well as NO released via various mechanisms that include hemichannel-related pathways. Below we discuss the present findings in the perspective of this Ca²⁺ signal communication framework.

Irradiation had dose-dependent effects on γ-H2AX scoring in the directly irradiated zone, while a saturation effect was observed in the bystander area (FIG. 169 and FIG. 170), as reported by others. Saturation effects in the bystander zone were also observed in the [Ca²⁺ ]ᵢ responses of recipient cells to medium transfer collected from irradiated cells (FIG. 192 to FIG. 195), as observed with γ-rays; superoxide production triggered by α-particles likewise displayed saturation effects. Saturation may result from rate-limiting processes in the signaling cascade and/or regenerative mechanisms that generate a maximal output in response to a variable input. In line with this, regenerative mechanisms play a prominent role in long-range Cx-based intercellular Ca²⁺signal communication, involving intracellular IP₃ regeneration via Ca²⁺ activation of PLC or ATP-induced release of extracellular ATP.

IP₃ diffusion through gap junctions was found to contribute to bystander signaling as demonstrated by the reduced γ-H2AX spreading in C6 glioma cells expressing IP₃-impermeable Cx26 channels compared to those expressing WT Cx26; gap junctional coupling was furthermore maintained during the time frame of bystander spreading (FIG. 233). Cx43 hemichannels, which are normally closed, were open 5 min post-irradiation, as supported by ATP release, PI dye uptake and single channel patch clamp experiments (FIG. 181 to FIG. 191). Connexin-linked ATP release triggered by γ-rays has been reported in various cell types based on work with non-specific blockers like lindane or more specific connexin-targeting peptides like Gap26. In vivo evidence from Cx43 heterozygous mice (Cx43^{+/-}) exposed to X-rays has furthermore demonstrated a strong linkage between connexin-based ATP release and γ-H2AX bystander responses. Here, we report that Cbx, Gap26 and TAT-Gap19, which inhibits Cx43-based hemichannels but not gap junctions, suppressed γ-H2AX appearance in the bystander zone, with stronger effect size and statistical significance as compared to the irradiated zone (FIG. 175-FIG. 178). The more limited effect of these agents in the irradiated zone is expected, as the direct cell irradiation in this zone intrinsically reduces the contribution of bystander signaling. Surprisingly, endothelial cells isolated from C57Bl/6 Cx43″:Tie2-Cre mice demonstrated non-significant effects on bystander communication. However, Cx37 and Cx40 are still present in these KO animals and we observed increased expression of Cx37 which may explain the rather limited bystander response (FIG. 35b). As mentioned by Kwak et al, it is possible that Cx43 interacts with NO resulting in a disturbed NO signaling pathway in Cx43 KO cells. This interaction has however not been proven yet. On the contrary the interaction of Cx37 with eNOS has been shown to result in an increased NO production in Cx37 knockdown cells.

The irradiated cells showed clear oscillatory [Ca²⁺ ]ᵢ dynamics that play a role in hemichannel opening and ATP release. Blocking connexin channels furthermore strongly inhibited the [Ca²⁺]1 transient induced by medium transfer from irradiated cultures to recipient naive cultures (FIG. 200 and FIG. 201), when applied to irradiated dishes but also recipient dishes. This suggests that connexins, presumably hemichannels, facilitate the paracrine release of agents that activate [Ca²⁺ ]ᵢ changes in recipient cells, but also play a role in Ca²⁺ entry at the recipient side. ROS-induced Ca²⁺ entry has indeed been demonstrated to be an important factor in the generation of bystander effects. In addition to ATP, the directly irradiated cells also released ROS (FIG. 202-FIG. 206), which has been suggested to act as a trigger for ATP release; accordingly, ROS inhibition with NALC suppressed ATP release ((FIG. 212 and FIG. 213). Moreover, NALC inhibition in irradiated cell dishes and PPADS purinergic receptor inhibition in recipient dishes suppressed [Ca²⁺ ]ᵢ changes in recipient cells after medium transfer (FIG. 200 and FIG. 201). ROS can influence [Ca²⁺ ]ᵢ signaling in several ways, by inducing ER Ca²⁺ release, inhibition of plasma membrane/ER Ca²⁺-ATPases, stimulation of Ca²⁺-induced Ca²⁺ release or mitochondrial permeability transition pore opening. Conversely, Ca²⁺ influences ROS signaling in two opposite ways: it induces secondary ROS generation in mitochondria via increased oxidative phosphorylation or mitochondrial permeability transition pore opening, but can also mitigate ROS signaling by activating anti-oxidant enzymes such as catalase and superoxide dismutase. Taken together, these data indicate a tightly connected ATP-ROS-[Ca²⁺ ]ᵢ signaling triad with strong impact on bystander γ-H2AX appearance as judged from the suppressive effects of PPADS, NALC, BAPTA-AM [Ca²⁺ ]ᵢ buffering and xestospongin-C inhibition of IP₃ receptors ((FIG. 214 to FIG. 220). NO may be part of the ATP-ROS-[Ca²⁺]1 signaling network, as it can be activated by Ca²⁺-calmodulin signaling, may facilitate intercellular Ca²⁺ signal communication, contributes to bystander [Ca²⁺ ]ᵢ signaling in photodynamic therapy and may induce DSBs via peroxynitrite (ONOO⁻) formed by its reaction with ROS. In line with this, C-PTIO-based NO scavenging inhibited radiation-induced ATP release (FIG. 30e) as well as bystander γ-H2AX generation (FIG. 209). Interestingly, the effects of C-PTIO were, like BAPTA-AM and NALC, stronger in pBMECs compared to RBE4, bringing down the γ-H2AX counts to almost zero (FIG. 208-FIG. 211 FIG. 30a-d); equally strong effects were observed for Gap26 (FIG. 175-FIG. 178).

All things considered, we propose that irradiation activates ATP-ROS-[Ca²⁺ ]ᵢ signaling with ROS as the primary generated signal, which, given its very short lifetime (10⁻⁹ sec for the hydroxyl radical) and diffusion distance (4 nm for the hydroxyl radical), has a limited role in long-range bystander consequences. Our results indicate that Ca²⁺ acts as an intracellular and ATP as an extracellular propagator of bystander effects. Both propagators diffuse but may also be actively regenerated, by Ca²⁺ activation of PLCs (e.g. PLCδ) followed by IP₃ generation and subsequent store Ca²⁺ release, or by ATP-induced ATP release in Ca²⁺-dependent or independent ways. NO, which has an estimated diffusion distance in the order of 160 μm, may be involved in propagation but this needs to be balanced with the fact that low NO concentrations may also mitigate bystander effects. During propagation, extensive interactions will occur between these messengers as delineated higher, effectively assembling a robust signaling network governed by multiple actors inside and outside the cells linked by plasma membrane connexin channels either as hemichannels or gap junctions (FIG. 221). Collectively, the data indicate feed-forward propagation of secondary ROS generation in the bystander zone, driven by the IP₃/Ca²⁺ signaling axis and leading to DNA damage, as exemplified by bystander zone interference with ROS/IP₃/Ca²⁺ signaling (FIG. 220). Mitochondria likely contribute to such secondary ROS generation, as documented for bystander communication of DNA mutations, autophagy and apoptosis, which may involve Ca²⁺-dependent mitochondrial permeability transition. Ca²⁺ signaling may, by itself, add to the picture by exerting calmodulin-dependent effects on chromatin structure.

### Example 4: Knock-Down of Cx43 in Vascular Endothelial Cells Prevents Blood-Brain Barrier Leakage Induced by In Vivo Exposure to Ionizing Radiation

The blood-brain barrier is an essential part of the brain that shields the parenchymal neural tissue from the blood to create a well-controlled interstitial milieu that is adapted for optimal neuronal and glial functioning. It is formed by an extremely dense capillary network of brain blood vessels at the capillary level, in such a way that almost every neuron has a local capillary blood-brain barrier interface in its microenvironment. The blood-brain barrier shielding and brain-protecting functions are altered/disturbed in almost every brain disease, making it an essential player contributing to the pathophysiology of brain disease. Importantly, ionizing radiation induces breakdown of the blood-brain barrier through its effects on brain microvascular endothelial cells which are the most vulnerable cells in the brain in terms of their sensitivity to irradiation. As a result, disruption of the blood-brain barrier endothelial cell function is one of several pathophysiological elements considered to contribute to the cognitive alterations that occur after brain irradiation (e.g. in the context of brain tumor therapy). Here, we demonstrate that endothelial Cx43 knock-out prevents radiation-induced blood-brain barrier leakage. We performed in vivo irradiation experiments whereby a single brain hemisphere was irradiated with X-rays at a dose of 20 Gy. At various time points after irradiation, in a time window of up to 96 h, we injected 3 and 10 kDa fluorescent tracers after which the animals were sacrificed (tracers were 3 kDa dextran fluorescein and 10 kDa dextran Texas red). Then, we determined the leakage of the vascular fluorescent markers in the brain cortex. We found that the two fluorescent markers significantly leaked through the blood-brain barrier and appeared in the cortical parenchyma starting from 30 min up to 48 h after irradiation (FIG. 236). Two clear peaks were discerned, one at 1 h after irradiation and another at 48 h post irradiation. Next we performed experiments in C57BL/6 Cx43fl/fl: Tie2-Cre mice that have targeted Cx43 knockdown in endothelial and hematopoetic cells under control of the Tie2 promoter and used the 1 h time point to verify the effect of irradiation (20 Gy) on blood-brain barrier leakage. Most interestingly, we found that leakage was completely suppressed in this case, demonstrating that absence of endothelial Cx43 reduces blood-brain barrier dysfunction after irradiation.

It is to be understood that although preferred embodiments, specific constructions and configurations, as well as materials, have been discussed herein for products according to the present invention, various changes or modifications in form and detail may be made without departing from the scope and technical teachings of this invention. For example, any formulas given above are merely representative of procedures that may be used. Functionality may be added or deleted from the block diagrams and operations may be interchanged among functional blocks. Steps may be added or deleted to methods described within the scope of the present invention.

1. An agent for inhibiting a connexin protein, connexin hemichannel or connexin gap junction, for use in the therapy of one or more ionizing radiation-induced disorders.

2-11. (canceled)

12. A pharmaceutical composition comprising an agent as defined in claim 1.

13. The pharmaceutical composition according to claim 12, further comprising a pharmaceutically acceptable carrier.

14. A method of therapy of an ionizing radiation-induced disorder, comprising:
a. inhibiting a connexin protein, connexin hemichannel or connexin gap junction in a subject, and
b. exposing the subject to radiation;
wherein step a is performed during and/or after step b.

15. The method according to claim 14, wherein the ionizing radiation-induced disorder is a cardiovascular disorder, a neurovascular disorder or a neurodegenerative disorder.

16. The method according to claim 15, wherein the ionizing radiation-induced disorder is an ionizing radiation-induced atherosclerosis.

17. The method according to claim 14, wherein the therapy is for reducing side-effects from a radiotherapy.

18. The method according to claim 17, wherein the radiotherapy is a thoracic radiotherapy or a head-and/or-neck radiotherapy.

19. The method according to claim 14, wherein
the connexin protein is a Cx43, and/or
the connexin hemichannel is a Cx43 hemichannel, and/or
the connexin gap junction is a Cx43 gap junction.

20. The method according to claim 14, wherein step a comprises inhibiting the connexin hemichannel in a specific manner with respect to a corresponding connexin gap junction.

21. The method according to claim 14, wherein step a comprises administering an agent for inhibiting the connexin protein, connexin hemichannel or connexin gap junction.

22. The method according to claim 21, wherein the agent is a connexin-targeting molecule or a hemichannel inhibitor.

23. The method according to claim 22, wherein the agent is a Gap19-based compound, an L2-based compound or a peptide5-based compound.

24. The method according to claim 14, wherein step a comprises administering a pharmaceutical composition comprising an agent for inhibiting the connexin protein, connexin hemichannel or connexin gap junction.

25. The method according to claim 24, wherein the pharmaceutical composition further comprises a pharmaceutically acceptable carrier.

26. The method according to claim 24, wherein the agent is a connexin-targeting molecule or a hemichannel inhibitor.

27. The method according to claim 26, wherein the agent is a Gap19-based compound, an L2-based compound or a peptide5-based compound.

28. A method of therapy of an atherosclerosis, comprising:
a. inhibiting a connexin hemichannel in a subject.

29. The method according to claim 28, wherein the connexin hemichannel is a Cx43 hemichannel.

30. The method according to claim 28, wherein step a comprises administering an agent for inhibiting the connexin hemichannel.
